SEQUENCE LISTING (54 sequences)

SEQ ID NO: 1 - protein, 574 aa, Human respiratory syncytial virus A2
  misc_feature (1)..(574): RSV F protein from A2 strain (full length)
MELLILKANAITTILTAVTFCFASGQNITEEFYQSTCSAVSKGYLSALRTGWYTSVITIELSNIKENKCNGTDAKVKLIKQELDKYKNAVTELQLLMQSTPPTNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEINLCNVDIFNPKYDCKIMTSKTDVSSSVITSLGAIVSCYGKTKCTASNKNRGIIKTFSNGCDYVSNKGMDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 2 - protein, 524 aa, Human respiratory syncytial virus A2
  misc_feature (1)..(524): RSV F protein (C-terminal truncation aa 1-524, derived from A2 strain)
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTN

SEQ ID NO: 3 - protein, 513 aa, Human respiratory syncytial virus A2
  misc_feature (1)..(513): RSV F protein (C-terminal truncation aa 1-513, derived from A2 strain)
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELL

SEQ ID NO: 4 - protein, 488 aa, Human respiratory syncytial virus A2
  misc_feature (1)..(488): RSV F protein truncated for fusion (derived from A2 strain)
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELL

SEQ ID NO: 5 - protein, 388 aa, Human respiratory syncytial virus A2
  misc_feature (1)..(388): RSV F1 protein (derived from A2 strain) (aa. 137-524)
FLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTN

SEQ ID NO: 6 - protein, 377 aa, Human respiratory syncytial virus A2
  misc_feature (1)..(377): RSV F1 protein (derived from A2 strain) (aa. 137-513)
FLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELL

SEQ ID NO: 7 - protein, 574 aa, Human respiratory syncytial virus
  misc_feature (1)..(574): RSV F protein from 18537 strain
MELLIHRSSAIFLTLAVNALYLTSSQNITEEFYQSTCSAVSRGYFSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALLSTNKAVVSLSNGVSVLTSKVLDLKNYINNRLLPIVNQQSCRISNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTNIMITTIIIVIIVVLLSLIAIGLLLYCKAKNTPVTLSKDQLSGINNIAFSK

SEQ ID NO: 8 - protein, 524 aa, Human respiratory syncytial virus
  misc_feature (1)..(524): RSV F protein from 18537 strain (C-terminal truncation, a.a.1-524)
MELLIHRSSAIFLTLAVNALYLTSSQNITEEFYQSTCSAVSRGYFSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALLSTNKAVVSLSNGVSVLTSKVLDLKNYINNRLLPIVNQQSCRISNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTN

SEQ ID NO: 9 - protein, 513 aa, Human respiratory syncytial virus
  misc_feature (1)..(513): RSV F protein from 18537 strain (C-terminal truncation, a.a.1-513)
MELLIHRSSAIFLTLAVNALYLTSSQNITEEFYQSTCSAVSRGYFSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALLSTNKAVVSLSNGVSVLTSKVLDLKNYINNRLLPIVNQQSCRISNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELL

SEQ ID NO: 10 - protein, 488 aa, Human respiratory syncytial virus
  misc_feature (1)..(488): RSV F protein truncated for fusion (derived from 18537 strain)
QNITEEFYQSTCSAVSRGYFSALRTGWYTSVITIELSNIKETKCNGTDTKVKLIKQELDKYKNAVTELQLLMQNTPAANNRARREAPQYMNYTINTTKNLNVSISKKRKRRFLGFLLGVGSAIASGIAVSKVLHLEGEVNKIKNALLSTNKAVVSLSNGVSVLTSKVLDLKNYINNRLLPIVNQQSCRISNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELL

SEQ ID NO: 11 - protein, 388 aa, Human respiratory syncytial virus
  misc_feature (1)..(388): RSV F1 protein (derived from 18537 strain) (aa. 137-524)
FLGFLLGVGSAIASGIAVSKVLHLEGEVNKIKNALLSTNKAVVSLSNGVSVLTSKVLDLKNYINNRLLPIVNQQSCRISNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELLHNVNTGKSTTN

SEQ ID NO: 12 - protein, 377 aa, Human respiratory syncytial virus
  misc_feature (1)..(377): RSV F1 protein (derived from 18537 strain; C-terminal truncation)(aa. 137-513)
FLGFLLGVGSAIASGIAVSKVLHLEGEVNKIKNALLSTNKAVVSLSNGVSVLTSKVLDLKNYINNRLLPIVNQQSCRISNIETVIEFQQMNSRLLEITREFSVNAGVTTPLSTYMLTNSELLSLINDMPITNDQKKLMSSNVQIVRQQSYSIMSIIKEEVLAYVVQLPIYGVIDTPCWKLHTSPLCTTNIKEGSNICLTRTDRGWYCDNAGSVSFFPQADTCKVQSNRVFCDTMNSLTLPSEVSLCNTDIFNSKYDCKIMTSKTDISSSVITSLGAIVSCYGKTKCTASNKNRGIIKTFSNGCDYVSNKGVDTVSVGNTLYYVNKLEGKNLYVKGEPIINYYDPLVFPSDEFDASISQVNEKINQSLAFIRRSDELL

SEQ ID NO: 13 - protein, 167 aa, Helicobacter pylori
  misc_feature (1)..(167): Ferritin (H. pylori J99)
MLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 14 - protein, 166 aa, Helicobacter pylori
  misc_feature (1)..(166): Ferritin (H. pylori J99) fragment (lacking C-terminal serine residue)
MLSKDIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRK

SEQ ID NO: 15 - protein, 183 aa, Homo sapiens
  misc_feature (1)..(183): Ferritin heavy chain
MTTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 16 - protein, 163 aa, Helicobacter pylori
  misc_feature (1)..(163): Ferritin (H. pylori) truncated for fusion (amino acids 5-167 of full length H. pylori ferritin)
DIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 17 - protein, 162 aa, Helicobacter pylori
  misc_feature (1)..(162): Ferritin (H. pylori) truncated for fusion (amino acids 5-166 of full length H. pylori ferritin)
DIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRK

SEQ ID NO: 18 - protein, 658 aa, Artificial Sequence
  Synthetic Truncated F glycoprotein linked to ferritin (1)
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGGSGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 19 - protein, 657 aa, Artificial Sequence
  Synthetic Truncated F glycoprotein linked to ferritin (2)
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGSGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 20 - protein, 656 aa, Artificial Sequence
  Synthetic Truncated F glycoprotein linked to ferritin (3)
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 21 - protein, 655 aa, Artificial Sequence
  Synthetic Truncated F glycoprotein linked to ferritin (4)
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 22 - protein, 622 aa, Artificial Sequence
  Synthetic Truncated F glycoprotein linked to sHSP20 (1)
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGSGSGSGSGTGTTMIQSSTGIQISGKGFMPISIIEGDQHIKVIAWLPGVNKEDIILNAVGDTLEIRAKRSPLMITESERIIYSEIPEEEEIYRTIKLPATVKEENASAKFENGVLSVILPKAESSIKKGINIE

SEQ ID NO: 23 - protein, 621 aa, Artificial Sequence
  Synthetic Truncated F glycoprotein linked to HSP (2)
QNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGSGSGSGSTGTTMIQSSTGIQISGKGFMPISIIEGDQHIKVIAWLPGVNKEDIILNAVGDTLEIRAKRSPLMITESERIIYSEIPEEEEIYRTIKLPATVKEENASAKFENGVLSVILPKAESSIKKGINIE

SEQ ID NO: 24 - protein, 683 aa, Artificial Sequence
  Synthetic F glycoprotein (w/N-terminal sequence) linked to ferritin (1)
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGGSGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 25 - protein, 682 aa, Artificial Sequence
  Synthetic F glycoprotein (w/N-terminal sequence) linked to ferritin (2)
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGSGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 26 - protein, 681 aa, Artificial Sequence
  Synthetic F glycoprotein (w/N-terminal sequence) linked to ferritin (3)
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 27 - protein, 680 aa, Artificial Sequence
  Synthetic F glycoprotein (w/N-terminal sequence) linked to ferritin (4)
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 28 - protein, 647 aa, Artificial Sequence
  Synthetic F glycoprotein (w/N-terminal sequence) linked to sHSP20 (1)
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGSGSGSGSGTGTTMIQSSTGIQISGKGFMPISIIEGDQHIKVIAWLPGVNKEDIILNAVGDTLEIRAKRSPLMITESERIIYSEIPEEEEIYRTIKLPATVKEENASAKFENGVLSVILPKAESSIKKGINIE

SEQ ID NO: 29 - protein, 646 aa, Artificial Sequence
  Synthetic F glycoprotein (w/N-terminal sequence) linked to HSP (2)
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGSGSGSGSTGTTMIQSSTGIQISGKGFMPISIIEGDQHIKVIAWLPGVNKEDIILNAVGDTLEIRAKRSPLMITESERIIYSEIPEEEEIYRTIKLPATVKEENASAKFENGVLSVILPKAESSIKKGINIE

SEQ ID NO: 30 - protein, 706 aa, Artificial Sequence
  Synthetic Leader-RSVF-linker-HypF (1)
MPMGSLQPLATLYLLGMLVASCLGMELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGGSGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRK

SEQ ID NO: 31 - protein, 705 aa, Artificial Sequence
  Synthetic Leader-RSVF-linker-HypF (2)
MPMGSLQPLATLYLLGMLVASCLGMELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGSGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRK

SEQ ID NO: 32 - protein, 704 aa, Artificial Sequence
  Synthetic Leader-RSVF-linker-HypF (3)
MPMGSLQPLATLYLLGMLVASCLGMELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRK

SEQ ID NO: 33 - protein, 703 aa, Artificial Sequence
  Synthetic Leader-full length RSVF-linker-HypF (4)
MPMGSLQPLATLYLLGMLVASCLGMELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRK

SEQ ID NO: 34 - protein, 671 aa, Artificial Sequence
  Synthetic Leader-RSVF-linker-sHSP20 (1)
MPMGSLQPLATLYLLGMLVASCLGMELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGSGSGSGSGTGTTMIQSSTGIQISGKGFMPISIIEGDQHIKVIAWLPGVNKEDIILNAVGDTLEIRAKRSPLMITESERIIYSEIPEEEEIYRTIKLPATVKEENASAKFENGVLSVILPKAESSIKKGINIE

SEQ ID NO: 35 - protein, 670 aa, Artificial Sequence
  Synthetic Leader-RSVF-linker-sHSP20 (2)
MPMGSLQPLATLYLLGMLVASCLGMELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGSGSGSGSTGTTMIQSSTGIQISGKGFMPISIIEGDQHIKVIAWLPGVNKEDIILNAVGDTLEIRAKRSPLMITESERIIYSEIPEEEEIYRTIKLPATVKEENASAKFENGVLSVILPKAESSIKKGINIE

SEQ ID NO: 36 - protein, 147 aa, Methanocaldococcus jannaschii DSM 2661
  misc_feature (1)..(147): sHSP20 full length
MFGRDPFDSLFERMFKEFFATPMTGTTMIQSSTGIQISGKGFMPISIIEGDQHIKVIAWLPGVNKEDIILNAVGDTLEIRAKRSPLMITESERIIYSEIPEEEEIYRTIKLPATVKEENASAKFENGVLSVILPKAESSIKKGINIE

SEQ ID NO: 37 - protein, 124 aa, Methanocaldococcus jannaschii DSM 2661
  misc_feature (1)..(124): sHSP20 truncated for fusion (amino acids 24-147 of full length sHSP20)
TGTTMIQSSTGIQISGKGFMPISIIEGDQHIKVIAWLPGVNKEDIILNAVGDTLEIRAKRSPLMITESERIIYSEIPEEEEIYRTIKLPATVKEENASAKFENGVLSVILPKAESSIKKGINIE

SEQ ID NO: 38 - protein, 573 aa, Homo sapiens
  misc_feature (1)..(573): HSP60 (human)
MLRLPTVFRQMRPVSRVLAPHLTRAYAKDVKFGADARALMLQGVDLLADAVAVTMGPKGRTVIIEQSWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLTTAEVVVTEIPKEEKDPGMGAMGGMGGGMGGGMF

SEQ ID NO: 39 - protein, 840 aa, Homo sapiens
  misc_feature (1)..(840): HSP70 (human)
MSVVGIDLGFQSCYVAVARAGGIETIANEYSDRCTPACISFGPKNRSIGAAAKSQVISNAKNTVQGFKRFHGRAFSDPFVEAEKSNLAYDIVQLPTGLTGIKVTYMEEERNFTTEQVTAMLLSKLKETAESVLKKPVVDCVVSVPCFYTDAERRSVMDATQIAGLNCLRLMNETTAVALAYGIYKQDLPALEEKPRNVVFVDMGHSAYQVSVCAFNRGKLKVLATAFDTTLGGRKFDEVLVNHFCEEFGKKYKLDIKSKIRALLRLSQECEKLKKLMSANASDLPLSIECFMNDVDVSGTMNRGKFLEMCNDLLARVEPPLRSVLEQTKLKKEDIYAVEIVGGATRIPAVKEKISKFFGKELSTTLNADEAVTRGCALQCAILSPAFKVREFSITDVVPYPISLRWNSPAEEGSSDCEVFSKNHAAPFSKVLTFYRKEPFTLEAYYSSPQDLPYPDPAIAQFSVQKVTPQSDGSSSKVKVKVRVNVHGIFSVSSASLVEVHKSEENEEPMETDQNAKEEEKMQVDQEEPHVEEQQQQTPAENKAESEEMETSQAGSKDKKMDQPPQAKKAKVKTSTVDLPIENQLLWQIDREMLNLYIENEGKMIMQDKLEKERNDAKNAVEEYVYEMRDKLSGEYEKFVSEDDRNSFTLKLEDTENWLYEDGEDQPKQVYVDKLAELKNLGQPIKIRFQESEERPKLFEELGKQIQQYMKIISSFKNKEDQYDHLDAADMTKVEKSTNEAMEWMNNKLNLQNKQSLTMDPVVKSKEIEAKIKELTSTCSPIISKPKPKVEPPKEEQKNAEQNGPVDGQGDNPGPQAAEQGTDTAVPSDSDKKLPEMDID

SEQ ID NO: 40 - protein, 854 aa, Homo sapiens
  misc_feature (1)..(854): HSP90 alpha isoform 1
MPPCSGGDGSTPPGPSLRDRDCPAQSAEYPRDRLDPRPGSPSEASSPPFLRSRAPVNWYQEKAQVFLWHLMVSGSTTLLCLWKQPFHVSAFPVTASLAFRQSQGAGQHLYKDLQPFILLRLLMPEETQTQDQPMEEEEVETFAFQAEIAQLMSLIINTFYSNKEIFLRELISNSSDALDKIRYESLTDPSKLDSGKELHINLIPNKQDRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPITLFVEKERDKEVSDDEAEEKEDKEEEKEKEEKESEDKPEIEDVGSDEEEEKKDGDKKKKKKIKEKYIDQEELNKTKPIWTRNPDDITNEEYGEFYKSLTNDWEDHLAVKHFSVEGQLEFRALLFVPRRAPFDLFENRKKKNNIKLYVRRVFIMDNCEELIPEYLNFIRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHIYYITGETKDQVANSAFVERLRKHGLEVIYMIEPIDEYCVQQLKEFEGKTLVSVTKEGLELPEDEEEKKKQEEKKTKFENLCKIMKDILEKKVEKVVVSNRLVTSPCCIVTSTYGWTANMERIMKAQALRDNSTMGYMAAKKHLEINPDHSIIETLRQKAEADKNDKSVKDLVILLYETALLSSGFSLEDPQTHANRIYRMIKLGLGIDEDDPTADDTSAAVTEEMPPLEGDDDTSRMEEVD

SEQ ID NO: 41 - protein, 732 aa, Homo sapiens
  misc_feature (1)..(732): HSP90 alpha isoform 2
MPEETQTQDQPMEEEEVETFAFQAEIAQLMSLIINTFYSNKEIFLRELISNSSDALDKIRYESLTDPSKLDSGKELHINLIPNKQDRTLTIVDTGIGMTKADLINNLGTIAKSGTKAFMEALQAGADISMIGQFGVGFYSAYLVAEKVTVITKHNDDEQYAWESSAGGSFTVRTDTGEPMGRGTKVILHLKEDQTEYLEERRIKEIVKKHSQFIGYPITLFVEKERDKEVSDDEAEEKEDKEEEKEKEEKESEDKPEIEDVGSDEEEEKKDGDKKKKKKIKEKYIDQEELNKTKPIWTRNPDDITNEEYGEFYKSLTNDWEDHLAVKHFSVEGQLEFRALLFVPRRAPFDLFENRKKKNNIKLYVRRVFIMDNCEELIPEYLNFIRGVVDSEDLPLNISREMLQQSKILKVIRKNLVKKCLELFTELAEDKENYKKFYEQFSKNIKLGIHEDSQNRKKLSELLRYYTSASGDEMVSLKDYCTRMKENQKHIYYITGETKDQVANSAFVERLRKHGLEVIYMIEPIDEYCVQQLKEFEGKTLVSVTKEGLELPEDEEEKKKQEEKKTKFENLCKIMKDILEKKVEKVVVSNRLVTSPCCIVTSTYGWTANMERIMKAQALRDNSTMGYMAAKKHLEINPDHSIIETLRQKAEADKNDKSVKDLVILLYETALLSSGFSLEDPQTHANRIYRMIKLGLGIDEDDPTADDTSAAVTEEMPPLEGDDDTSRMEEVD

SEQ ID NO: 42 - protein, 633 aa, Homo sapiens
  misc_feature (1)..(633): HSP100 (human) (NP_006651.2)
MPSCGACTCGAAAVRLITSSLASAQRGISGGRIHMSVLGRLGTFETQILQRAPLRSFTETPAYFASKDGISKDGSGDGNKKSASEGSSKKSGSGNSGKGGNQLRCPKCGDLCTHVETFVSSTRFVKCEKCHHFFVVLSEADSKKSIIKEPESAAEAVKLAFQQKPPPPPKKIYNYLDKYVVGQSFAKKVLSVAVYNHYKRIYNNIPANLRQQAEVEKQTSLTPRELEIRRREDEYRFTKLLQIAGISPHGNALGASMQQQVNQQIPQEKRGGEVLDSSHDDIKLEKSNILLLGPTGSGKTLLAQTLAKCLDVPFAICDCTTLTQAGYVGEDIESVIAKLLQDANYNVEKAQQGIVFLDEVDKIGSVPGIHQLRDVGGEGVQQGLLKLLEGTIVNVPEKNSRKLRGETVQVDTTNILFVASGAFNGLDRIISRRKNEKYLGFGTPSNLGKGRRAAAAADLANRSGESNTHQDIEEKDRLLRHVEARDLIEFGMIPEFVGRLPVVVPLHSLDEKTLVQILTEPRNAVIPQYQALFSMDKCELNVTEDALKAIARLALERKTGARGLRSIMEKLLLEPMFEVPNSDIVCVEVDKEVVEGKKEPGYIRAPTKESSEEEYDSGVEEEGWPRQADAANS

SEQ ID NO: 43 - protein, 4 aa, Artificial Sequence
  Synthetic Linker
SGSG

SEQ ID NO: 44 - protein, 7 aa, Artificial Sequence
  Synthetic Linker
NGTGGSG

SEQ ID NO: 45 - protein, 5 aa, Artificial Sequence
  Synthetic Linker
SGGSG

SEQ ID NO: 46 - protein, 6 aa, Artificial Sequence
  Synthetic Linker
GGSGSG

SEQ ID NO: 47 - protein, 6 aa, Artificial Sequence
  Synthetic Linker
SGSGSG

SEQ ID NO: 48 - protein, 7 aa, Artificial Sequence
  Synthetic Linker
SGGSGSG

SEQ ID NO: 49 - protein, 9 aa, Artificial Sequence
  Synthetic Linker
SGSGSGSGS

SEQ ID NO: 50 - protein, 10 aa, Artificial Sequence
  Synthetic Linker
SGSGSGSGSG

SEQ ID NO: 51 - protein, 24 aa, Homo sapiens
  misc_feature (1)..(24): N-terminal leader (from human CD5)
MPMGSLQPLATLYLLGMLVASCLG

SEQ ID NO: 52 - protein, 20 aa, Artificial Sequence
  Synthetic N-terminal leader
METDTLLLWVLLLWVPGSTG

SEQ ID NO: 53 - protein, 21 aa, Artificial Sequence
  Synthetic N-terminal leader
MDSYLLMWGLLTFIMVPGCQA

SEQ ID NO: 54 - protein, 25 aa, Artificial Sequence
  Synthetic N-terminal F protein sequence
MELLILKANAITTILTAVTFCFASG

(19) United States
(12) Patent Application Publication
(10) Pub. No.: US 2015/0110825 A1
(43) Pub. Date: Apr. 23, 2015

(54) SELF-ASSEMBLED NANOPARTICLE VACCINES

(71) Applicant: Massachusetts Institute of Technology, Cambridge, MA (US)

(72) Inventors: Ram SASISEKHARAN, Cambridge, MA (US); Aditya RAGURAM, Cambridge, MA (US); Vidya SUBRAMANIAN, Waltham, MA (US)

(21) Appl. No.: 14/495,640

(22) Filed: Sep. 24, 2014

Related U.S. Application Data
(60) Provisional application No. 61/881,848, filed on Sep. 24, 2013.

(51) Int. Cl. A61K 39/155; A61K 47/48; C07K 14/00; C12N 7/00; C07K 14/205

(52) U.S. Cl. CPC A61K 39/155; C12N 7/00; C07K 14/205; C07K 14/00; A61K 47/48892; C12N 2760/18522; C12N 2760/18534; C12N 2760/18571; C07K 2319/00
USPC 424/186.1; 530/350; 536/23.4; 435/320.1; 435/252.3; 435/252.33; 435/348; 435/325; 435/365; 435/357; 435/367

(57) ABSTRACT

The present invention provides nanoparticles and compositions of various constructs that combine meta-stable viral proteins (e.g., RSV F protein) and self-assembling molecules (e.g., ferritin, HSPs) such that the pre-fusion conformational state of these key viral proteins is preserved (and locked) along with the protein self-assembling into a polyhedral shape, thereby creating nanoparticles that are effective vaccine agents. The invention also provides nanoparticles comprising a viral fusion protein, or fragment or variant thereof, and a self-assembling molecule, and immunogenic and vaccine compositions including the same.

### RELATED APPLICATIONS

This application claims priority to and the benefit of U.S. Provisional Patent Application No. 61/881,848, filed Sep. 24, 2013, the entire contents of which is herein incorporated by reference.

### BACKGROUND

Human respiratory syncytial virus (RSV) is a negative-sense, single-stranded RNA virus of the family Paramyxoviridae and a member of the paramyxovirus subfamily Pneumovirinae. RSV is a major cause of lower respiratory tract infections in young children, and often results in multiple hospital visits during the first few years of a child's life. In fact, because the protective immunity produced following natural infection with RSV wanes over time, it is possible to be infected with RSV multiple times and some infants can become infected with RSV more than once a season. Although prophylactic treatment for RSV in young children is available, previous efforts to produce a vaccine against RSV have been unsuccessful. Due to the complexity of RSV proteins, it is difficult to obtain homogeneous immunogenic preparations of the proteins. Accordingly, there is a need for improved RSV protein compositions and vaccines, and improved methods of producing the same.

### SUMMARY OF THE INVENTION

One of the major challenges to vaccine development for several viruses (e.g., RSV, Flaviviruses (e.g., Dengue, West Nile), and HIV) is to ‘capture’ their key viral protein in the ‘pre-fusion’ state which is critical as it ‘presents’ important residues on the viral protein that are critical for virus fusion with host cells. It is usually difficult to capture these viral proteins in this state, as they quickly fuse and undergo conformational changes. Thus, creating a mechanism that preserves and ‘locks’ the pre-fusion state of these ‘meta-stable’ viral proteins would enable the development of more effective vaccines against these viral agents. Accordingly, methods that allow for locking meta-stable viral proteins in their pre-fusion state are highly desirable.

The concept of self-assembling nanoparticles has been applied to create new vaccine technologies. Molecules such as ferritin and heat shock proteins (HSPs) are known to naturally assemble into polyhedral shapes. By genetically attaching a meta-stable viral proteins to a self-assembling protein, various polyhedral nanoparticles are created. A protein-based nanoparticle that incorporates meta-stable viral proteins in their pre-fusion states can be used as an effective vaccine against these viral agents. However, there are major limitations or challenges to the use self-assembling systems for meta-stable viral proteins as it is not obvious that just linking such proteins via their N or C terminus to a self-assembly molecule such as ferritin or a heat shock protein would preserve the proper orientation or lock the meta-stable viral protein conformation for effective vaccine design. Therefore, an important consideration in the creation of the meta-stable viral protein based nanoparticles is how to attach the meta-stable protein to the self-assembling molecule such that it does not interfere with the self-assembly of the molecule and lock the meta-stable protein in its pre-fusion conformation.

This invention outlines nanoparticles and compositions of various constructs that combine exemplary meta-stable viral proteins (e.g., RSV F protein) and self-assembling molecules (e.g., ferritin, HSPs) such that the pre-fusion conformational state of these key viral proteins is preserved (and locked) along with the protein self-assembling into a polyhedral shape, thereby creating nanoparticles that are effective vaccine agents.

The present invention, in general, relates to nanoparticles comprising a viral fusion protein, or fragment or variant thereof, and a self-assembling molecule, and immunogenic and vaccine compositions including the same.

Accordingly, in one aspect, the invention relates to a nanoparticle comprising a viral fusion protein, such as Respiratory syncytial virus (RSV) F protein, or fragment (e.g., truncation) thereof, and a self-assembling molecule, wherein the self-assembling molecule forms a polymeric assembly that captures the viral fusion protein (e.g., F protein) or fragment thereof in a meta-stable pre-fusion conformation, thereby forming the nanoparticle.

In some aspects, the invention relates to an immunogenic composition comprising a nanoparticle of the invention and a pharmaceutically acceptable carrier. In some embodiments, the immunogenic composition comprises an adjuvant.

In some aspects, the invention relates to a vaccine composition comprising a nanoparticle of the invention, wherein the viral fusion protein homotrimers, such as F protein homotrimers, in a pre-fusion conformation are displayed on the surface of a shell formed by polymeric assembly of the self-assembly molecule. In some embodiments, the vaccine comprises an adjuvant.

In some embodiments, the viral fusion protein is a class I, II or III fusion protein. In some embodiments, the viral fusion protein adopts a dimeric or trimeric quaternary structure.

In some embodiments, the viral fusion protein is a Paramyxoviridae, Flaviviridae, or Retroviridae viral fusion protein. In some embodiments, the Flavivirdae viral fusion protein is a Flavivirus. In some embodiments, the Flavivirus is West Nile virus, Dengue virus or yellow fever virus. In other embodiments, the virus is Dengue virus and the fusion protein is E protein. In some embodiments, the Paramyxoviridae viral fusion protein is a Paramyxovirinae or Pneumonvirinae virus, such as Avularvirus, Respirovirus, and Pneumovirus. In some embodiments, the virus is New Castle disease virus, Sendai virus, and Respiratory syncytial virus (RSV). In other embodiments, the virus is RSV and the fusion protein is F protein.

In some embodiments, the F protein, or fragment thereof, lacks the transmembrane domain and cytotail domain (amino acids 1-524; SEQ ID NO: 2). In some embodiments, the F protein, or fragment thereof, lacks the transmembrane domain and cytotail domain and a portion of the HRB domain (amino acids 1-513; SEQ ID NO: 3). In some embodiments, the F protein comprises an F1 and F2 heterodimer. In some embodiments, the F protein, or fragment thereof, comprises the F1 domain (e.g., from about amino acid 137 to about amino acid 524 (SEQ ID NO: 5), or from about amino acid 137 to about amino acid 513 (SEQ ID NO: 6)). In some embodiments, the F protein, or fragment thereof, comprises an F protein, or fragment thereof, without the N-terminal sequence MELLILKANAITTILTAVTFCFASG (SEQ ID NO: 54). In some embodiments, the F protein fragment comprises an ectodomain. In some embodiments, the F protein fragment comprises a heptad-repeat A domain (HRA) and a heptad-repeat C domain (HRC). In some embodiments, the F protein fragment comprises an HRA domain, an HRC domain, and F1 domains I and II. In some embodiments, the F protein fragment comprises an HRA domain, an HRC domain, F1 domains I and II, and a heptad-repeat B domain (HRB). In some embodiments, the nanoparticle comprises one or more homotrimers of F1 and F2.

In some embodiments, the F protein comprises an amino acid sequence set forth in SEQ ID NOs:1-12.

In some embodiments, the viral fusion protein, such as the F protein, is covalently attached to the self-assembling molecule.

In some embodiments, the viral fusion protein, such as the F protein (e.g., an F protein from SEQ ID NOs: 1-12), is genetically fused to the self-assembling molecule.

In some embodiments, the self-assembling molecule is proteinaceous or non-proteinaceous. In some embodiments, the self-assembling molecule is a protein, peptide, nucleic acid, a virus-like particle, a viral capsid, lipid, or carbohydrate. In some embodiments, the self-assembling molecule assembles into a shell with polyhedral symmetry, such as octahedral symmetry. In some embodiments, the shell comprises twenty four monomers of the self-assembling molecule.

In some embodiments, self-assembling molecule is ferritin, heat shock protein, Dsp, lumazine synthase or MrgA. In some embodiments, the ferritin protein comprises an amino acid sequence set forth in SEQ ID NOs:13-17. In some embodiments, the self-assembling molecule is a heat shock protein, such as sHSP (small heat shock protein), HSP100, HSP90, HSP70, and HSP60. In some embodiments, the heat shock protein comprises an amino acid sequence set forth in SEQ ID NOs:36-42.

In some embodiments, the viral fusion protein (e.g., F protein) and self-assembling molecule are attached by means of a linker, such as an amino acid linker (e.g., a gly-ser linker). In some embodiments, the linker is a (GlySer)n linker. In some embodiments, the linker is of sufficient length to prevent steric hindrance between the self-assembling molecule and the viral fusion protein (e.g., F protein). For example, in some embodiments, the linker is about 5 to 7 amino acids long. In some embodiments, the linker attachment point on the F protein is leucine at position 513 of SEQ ID NO: 1, and the linker attachment point on ferritin is aspartic acid at position 5 of SEQ ID NO: 13.

In some embodiments, the viral fusion protein (e.g., F protein) further comprises an N-terminal leader in order to facilitate effective secretion of recombinant proteins from transfected cells (e.g., 293 cells) into the culture medium. Non-limiting N-terminal leader sequences include those set forth in SEQ ID NOs: 51-53.

In another aspect, the invention relates to an RSV F protein-ferritin fusion protein comprising the amino acid sequence set forth in SEQ ID NOs: 18-21, 24-27, or 30-33.

In another aspect, the invention relates to RSV F protein-heat shock protein fusion protein comprising the amino acid sequence set forth in SEQ ID NOs: 22, 23, 28, 29, 34, or 35.

In another aspect, the invention relates to a method of producing an antibody which inhibits and/or prevents RSV infection comprising administering to a subject the nanoparticle, immunogenic composition, vaccine composition, or fusion protein of the invention. In some embodiments, the antibody is isolated from the subject.

In another aspect, the invention relates to a method of producing a vaccine against RSV, the method comprising a) expressing a complex comprising a monomeric self-assembly molecule and an RSV F protein under conditions such that F protein trimers in a pre-fusion conformation are displayed on the surface of a shell formed by polymerization of the self-assembly molecule, and b) recovering the shell displaying the F protein. In some embodiments, the subject is vaccinated against RSV with the vaccine of the present invention.

In yet another aspect, the invention relates to isolated nucleic acids encoding the nanoparticle or fusion protein of the invention, vectors comprising the nucleic acids, and isolated cells comprising the nucleic acids.

In yet another aspect, the invention relates to a kit comprising the nanoparticle or fusion protein of the invention and instructions for use.

### BRIEF DESCRIPTION OF THE FIGURES

FIG. 1 shows a schematic representation of the RSV F protein. The protein domains are designated in the figure.

FIG. 2 shows the structure of a ferritin shell (ferritin cage) composed of twenty-four ferritin monomers that occupy each of the twenty-four symmetry domains of an octahedral symmetry.

FIG. 3 shows the structure of a heat shock protein shell (HSP cage) composed of twenty-four monomers that self assemble into a shell with octahedral symmetry.

FIG. 4 shows the three “equivalent” points on a ferritin monomer. Connecting these points yields a triangle whose centroid lies on the three-fold axis of symmetry.

FIG. 5 shows the process of orienting the RSV F protein trimers and ferritin trimers along the same three-fold axis. Each RSV F protein monomer is linked at Leu513 to the Asp5 residue of a ferritin monomer. Connecting each of these sets of residues forms two equilateral triangles with side lengths of 12.1 angstroms for the RSV F protein trimer and 28.7 angstroms for the ferritin trimer.

FIG. 6A shows the structure of a RSV F protein monomer linked to a ferritin monomer via a linker. FIG. 6B shows a zoomed in version of the linker shown in FIG. 6A.

FIG. 7 shows an octahedral ferritin-F protein nanoparticle with twenty-four F proteins in total oriented about the three-fold axes.

FIG. 8 shows the amino acid sequence of an exemplary RSVF protein-linker-Helicobacter pylori ferritin (HypF) (“RSVF-HypF”) fusion protein (SEQ ID NO: 30). The leader sequence (a human CD5 leader sequence) is in regular font, the RSV F domain is bolded, the linker is in italics, and the HypF domain is bolded/underlined.

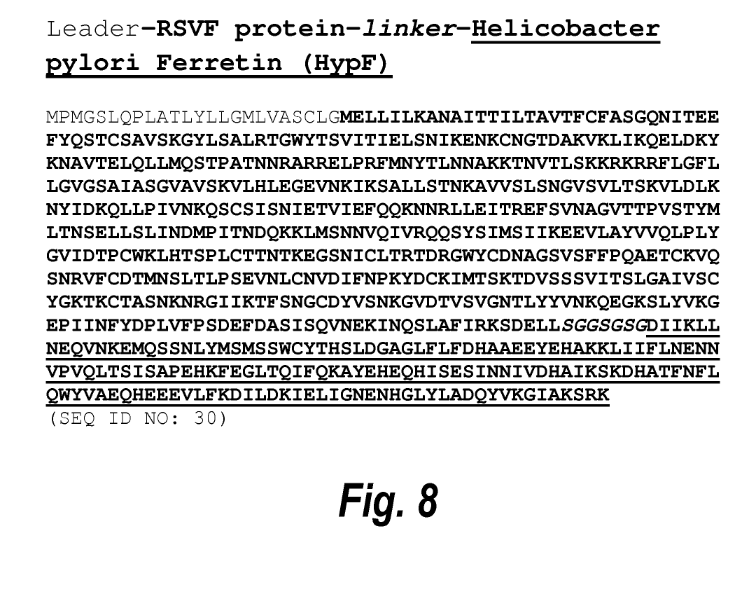

FIG. 9 shows the expression over time of a RSVF-HypF fusion protein in 293F cells plated at two different cell densities by Western blot. Supernatants were harvested on days 3, 4, 5, and 6. The D25 antibody, which specifically detects the pre-fusion state of the RSV F protein, was used for detection. The molecular weight suggests the band corresponds to a trimer and that the protein is expressed in the pre-fusion state.

FIG. 10 shows the purification of the RSVF-HypF fusion protein from FIG. 9. Proteins were eluted off the column using a NaCl gradient. All elution fractions were detected using the monoclonal D25 antibody.

### DETAILED DESCRIPTION

### Definitions

Terms used in the claims and specification are defined as set forth below unless otherwise specified. In the case of direct conflict with a term used in a parent provisional patent application, the term used in the instant specification shall control.

“Amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

An “amino acid substitution” refers to the replacement of at least one existing amino acid residue in a predetermined amino acid sequence (an amino acid sequence of a starting polypeptide) with a second, different “replacement” amino acid residue. An “amino acid insertion” refers to the incorporation of at least one additional amino acid into a predetermined amino acid sequence. While the insertion will usually consist of the insertion of one or two amino acid residues, the present larger “peptide insertions,” can be made, e.g. insertion of about three to about five or even up to about ten, fifteen, or twenty amino acid residues. The inserted residue(s) may be naturally occurring or non-naturally occurring as disclosed above. An “amino acid deletion” refers to the removal of at least one amino acid residue from a predetermined amino acid sequence.

“Polypeptide,” “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

“Nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985); and Cassol et al., 1992; Rossolini et al., Mol. Cell. Probes 8:91-98, 1994). For arginine and leucine, modifications at the second base can also be conservative. The term nucleic acid is used interchangeably with gene, cDNA, and mRNA encoded by a gene. Polynucleotides of the present invention can be composed of any polyribonucleotide or polydeoxribonucleotide, which can be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, the polynucleotide can be composed of triple-stranded regions comprising RNA or DNA or both RNA and DNA. A polynucleotide can also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

As used herein, the terms “linked,” “fused,” or “fusion,” in the context of joining together of two more elements or components or domains by whatever means including chemical conjugation or recombinant means (e.g., by genetic fusion) are used interchangably. Methods of chemical conjugation (e.g., using heterobifunctional crosslinking agents) are known in the art. More specifically, as used herein, “viral fusion protein-self-assembling molecule complex or fusion” refers to the genetic or chemical conjugation of a meta-stable viral fusion protein (e.g., RSV F protein) to a self-assembling molecule, which may or may not be proteinaceous. As used herein, “viral fusion protein-self-assembling molecule fusion protein” refers to the genetic or chemical conjugation of a meta-stable viral fusion protein (e.g., RSV F protein) to a proteinaceous self-assembling molecule (e.g., ferritin, HSP). In a preferred embodiment, the viral fusion protein is fused to a proteinaceous self-assembling molecule, such as ferritin or HSP, via a linker, such as a glycine-serine (gly-ser) linker.

As used herein, the term “gly-ser linker” refers to a peptide that consists of glycine and serine residues.

As used herein, the term “proline-alanine (pro-ala)” linker refers to a peptide that consists of proline and alanine residues.

A polypeptide or amino acid sequence “derived from” a designated polypeptide or protein refers to the origin of the polypeptide. Preferably, the polypeptide or amino acid sequence which is derived from a particular sequence has an amino acid sequence that is essentially identical to that sequence or a portion thereof, wherein the portion consists of at least 10-20 amino acids, preferably at least 20-30 amino acids, more preferably at least 30-50 amino acids, or which is otherwise identifiable to one of ordinary skill in the art as having its origin in the sequence.

Polypeptides derived from another peptide may have one or more mutations relative to the starting polypeptide, e.g., one or more amino acid residues which have been substituted with another amino acid residue or which has one or more amino acid residue insertions or deletions.

A polypeptide can comprise an amino acid sequence which is not naturally occurring. Such ‘variants’ necessarily have less than 100% sequence identity or similarity with the starting molecule. In a preferred embodiment, the variant will have an amino acid sequence from about 75% to less than 100% amino acid sequence identity or similarity with the amino acid sequence of the starting polypeptide, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant molecule.

In one embodiment, there is one amino acid difference between a starting polypeptide sequence and the sequence derived therefrom. Identity or similarity with respect to this sequence is defined herein as the percentage of amino acid residues in the candidate sequence that are identical (i.e., same residue) with the starting amino acid residues, after aligning the sequences and introducing gaps, if necessary, to achieve the maximum percent sequence identity.

In an embodiment, the peptides of the invention are encoded by a nucleotide sequence. Nucleotide sequences of the invention can be useful for a number of applications, including: cloning, gene therapy, protein expression and purification, mutation introduction, DNA vaccination of a host in need thereof, antibody generation for, e.g., passive immunization, PCR, primer and probe generation, and the like.

It will also be understood by one of ordinary skill in the art that the viral fusion protein or self-assembling proteins (an results protein fusions) may be altered such that they vary in sequence from the naturally occurring or native sequences from which they were derived, i.e., referred to as “variants,” while retaining the desirable activity (e.g., folding or self-assembly) of the native sequences. For example, nucleotide or amino acid substitutions leading to conservative substitutions or changes at “non-essential” amino acid residues may be made. Mutations may be introduced by standard techniques, such as site-directed mutagenesis and PCR-mediated mutagenesis.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in a binding polypeptide is preferably replaced with another amino acid residue from the same side chain family. In another embodiment, a string of amino acids can be replaced with a structurally similar string that differs in order and/or composition of side chain family members.

As used herein, the term “infectious agent” refers to microorganisms that cause an infection in a vertebrate. Usually, the organisms are viruses, bacteria, parasites, protozoa and/or fungi.

As used herein, the term “antigenic formulation” or “antigenic composition” or “immunogenic composition” refers to a preparation which, when administered to a vertebrate, especially a bird or a mammal, will induce an immune response.

Enveloped viruses penetrate the host cells by a process of fusion between the viral and cell membranes. This process is catalyzed by a fusiongenic activity of a viral surface glycoprotein. A characteristic feature of the fusion glycoproteins for many membrane-enveloped viruses is that they are synthesized as inactive precursors which undergo several post-transitional modifications to be displayed on virions in metastable forms.

As used herein, the term “meta-stable”, as used in the context of a protein (e.g., a viral fusion protein such as the RSV F protein), refers to a labile conformational state that rapidly converts to a more stable conformational state upon a change in conditions. For example, the pre-fusion RSV F protein is in a labile meta-stable conformation, and converts to the more stable post-fusion conformation upon, e.g., fusion to a host cell.

As used herein, the term “self-assembling molecule” refers to a molecule that undergoes spontaneous or induced assembly into defined, stable, noncovalently bonded assemblies that are held together by intermolecular forces. Self-assembling molecules include protein, peptides, nucleic acids, virus-like particles, lipids and carbohydrates. Non-limiting examples of self-assembling molecules include ferritin, heat shock protein, DSP, lumazine synthase, and DNA.

As used herein, the term “protein cage” or “protein shell’ refers to a composition of a proteinaceous shell that self-assembles to form a protein cage (a structure with an interior cavity which is either naturally accessible to the solvent or can be made to be so by altering solvent concentration, pH, equilibria ratios, etc.)

As used herein, the term “nanoparticle” encompasses a protein cage, but also includes cages that are not proteinaceous, which includes both the shell (e.g., protein cage) and the nanoparticle core, which may or may not be loaded with cargo (e.g., adjuvant, therapeutic agent, imaging agent).

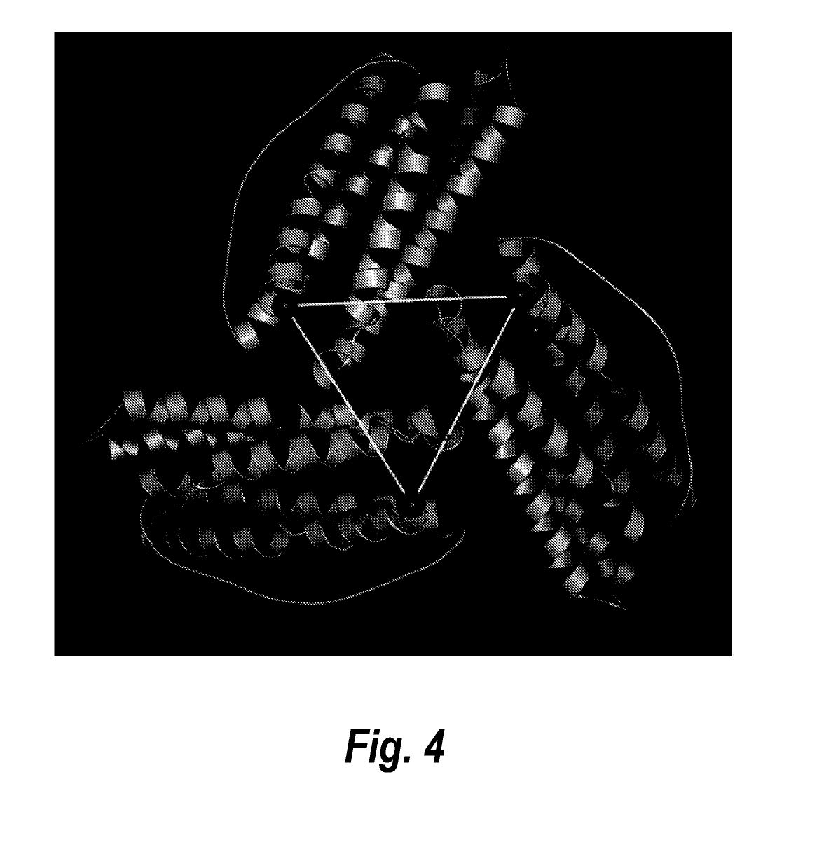

As used herein, the term “RSV F protein” and “F protein” and “Fusion protein” and “F protein polypeptide” and “Fusion protein polypeptide” are used interchangeably and refer to a polypeptide or protein having all or part of an amino acid sequence of an RSV Fusion protein polypeptide. Numerous RSV Fusion proteins, and variants (e.g., naturally occurring variants) have been described and are known to those of skill in the art (see, e.g., WO2008/114149 corresponding to US 20100203071, the contents of which are incorporated herein by reference). FIG. 4 of US 20100203071 shows an alignment of a number of RSV F protein variants which can be used in the present invention.

The term “pre-fusion,” as used in the context of an infectious agent, refers to the conformation of a protein of the infectious agent prior to fusion to a host cell. The term “post-fusion,” in this context, refers to the conformation of a protein of the infectious agent after fusion to a host cell.

The “post-fusion conformation” of RSV F protein is a trimer characterized by the presence of a six-helix bundle comprising 3 HRB and 3HRA regions. The post-fusion conformation of RSV F protein is typically characterized as having a “crutch” or “golf tee” conformation.

The “pre-fusion conformation” of RSV F protein is a conformation characterized by a trimer that contains a triple helix comprising 3 HRB regions. The post-fusion conformation of RSV F protein is typically characterized as having a “lollipop” or “ball and stem” conformation.

As used herein, “RSV F ecto-domain polypeptide” refers to an RSV F protein polypeptide that contains substantially the extracellular portion of mature RSV F protein, with or without the signal peptide (e.g., about amino acids 1 to about amino acid 524 (SEQ ID NO: 2), or about amino acid 22 to about amino acid 524), but lacks the transmembrane domain and cytoplasmic tail of naturally occurring RSV F protein. In some embodiments, the RSV F protein polypeptide contains substantially the extracellular portion of mature RSV F protein, with or without the signal peptide, and lacking the transmembrane domain, cytoplasmic tail, and part of the HRB domain (e.g., about amino acids 1 to about amino acid 513 (SEQ ID NO: 3), or about amino acid 22 to about amino acid 513, for example, 26-513 (SEQ ID NO: 4).

As used herein, “cleaved RSV F ecto-domain polypeptide” refers to a RSV F ectodomain polypeptide that has been cleaved at one or more positions from about 101/102 to about 160/161 to produce two subunits, in which one of the subunits comprises F₁ and the other subunit comprises F₂.

As used herein, “C-terminal uncleaved RSV F ecto-domain polypeptide” refers to an RSV F ectodomain polypeptide that is cleaved at one or more positions from about 101/102 to about 131/132, and is not cleaved at one or more positions from about 132/133 to about 160/161, to produce two subunits, in which one of the subunits comprises F₁ and the other subunit comprises F₂.

As used herein, “uncleaved RSV F ecto-domain polypeptide” refers to an RSV F ectodomain polypeptide that is not cleaved at one or more positions from about 101/102 to about 160/161. An uncleaved RSV F ecto-domain polypeptide can be, for example, a monomer or a trimer.

As used herein, the term “immunogens” or “antigens” refer to substances such as proteins, peptides, peptides, nucleic acids that are capable of eliciting an immune response. Both terms also encompass epitopes, and are used interchangeably.

As used herein, the term “vaccine” refers to a formulation which contains the fusion proteins or nanoparticles of the present invention, which is in a form that is capable of being administered to a vertebrate and which induces a protective immune response sufficient to induce immunity to prevent and/or ameliorate an infection and/or to reduce at least one symptom of an infection and/or to enhance the efficacy of another dose of the fusion proteins or nanoparticles. Typically, the vaccine comprises a conventional saline or buffered aqueous solution medium in which the composition of the present invention is suspended or dissolved. In this form, the composition of the present invention can be used conveniently to prevent, ameliorate, or otherwise treat an infection. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses. Vaccines can be administered in conjunction with an adjuvant.

As used herein, the term “adjuvant” refers to a compound that, when used in combination with a specific immunogen in a formulation, will augment or otherwise alter or modify the resultant immune response. Modification of the immune response includes intensification or broadening the specificity of either or both antibody and cellular immune responses. Modification of the immune response can also mean decreasing or suppressing certain antigen-specific immune responses.

As used herein, the term “pharmaceutically acceptable vaccine” refers to a formulation which contains a fusion protein, or nanoparticles of the present invention, which is in a form that is capable of being administered to a vertebrate and which induces a protective immune response sufficient to induce immunity to prevent and/or ameliorate an infection or disease, and/or to reduce at least one symptom of an infection or disease, and/or to enhance the efficacy of another dose of a fusion protein or nanoparticle. In one embodiment, to a formulation which contains a fusion protein, or nanoparticle, of the present invention. Typically, the vaccine comprises a conventional saline or buffered aqueous solution medium in which the composition of the present invention is suspended or dissolved. In this form, the composition of the present invention can be used conveniently to prevent, ameliorate, or otherwise treat an infection. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses.

As used herein, the term “ameliorating” refers to any therapeutically beneficial result in the treatment of a disease state, e.g., viral infection, including prophylaxis, lessening in the severity or progression, remission, or cure thereof.

As used herein, the term “in vivo” refers to processes that occur in a living organism.

The term “mammal” or “subject” or “patient” as used herein includes both humans and non-humans and include but is not limited to humans, non-human primates (e.g., chimpanzees, monkeys, baboons), canines, felines, mice, rats (e.g., cotton rats), bovines (e.g., calves), equines, porcines, guinea pigs, ferrets and hamsters.

As used herein, the term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information website.

A “therapeutic antibody” is an antibody, fragment of an antibody, or construct that is derived from an antibody, and can bind to a cell-surface antigen on a target cell to cause a therapeutic effect. Such antibodies can be chimeric, humanized or fully human antibodies. Methods are known in the art for producing such antibodies. Such antibodies include single chain Fc fragments of antibodies, minibodies and diabodies. Therapeutic antibodies may be monoclonal antibodies or polyclonal antibodies. In preferred embodiments, the therapeutic antibodies target viral fusion proteins (e.g., RSV F protein) in the pre-fusion conformation.

As used herein, the term “sufficient amount” or “amount sufficient to” means an amount sufficient to produce a desired effect, e.g., an amount sufficient to inhibit viral fusion to a cell.

As used herein, the term “therapeutically effective amount” is an amount that is effective to ameliorate a symptom of a disease. A therapeutically effective amount can be a “prophylactically effective amount” as prophylaxis can be considered therapy.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

It must be noted that, as used in the specification and the appended claims, the singular forms “a,” “an” and “the” include plural referents unless the context clearly dictates otherwise.

### I. Overview

The concept of self-assembling nanoparticles has been applied to create new vaccine technologies. Molecules such as ferritin and heat shock proteins (HSPs) are known to naturally assemble into polyhedral shapes. By genetically attaching a meta-stable viral protein to a self-assembling molecule, such as a protein or polypeptide, various polyhedral nanoparticles are created. A protein-based nanoparticle that incorporates meta-stable viral proteins in their pre-fusion states can be used as an effective vaccine against these viral agents. However, major limitations and challenges exist regarding the use of self-assembling systems for meta-stable viral proteins, as it is not obvious that just linking such proteins via their N or C terminus to self-assembling molecules such as ferritin or heat shock proteins would preserve the proper orientation or lock the meta-stable viral protein conformation for effective vaccine design. The present invention addresses important considerations in the creation of the meta-stable viral protein-based nanoparticles, in particular (1) how to attach the meta-stable protein to the self-assembling protein such that it does not interfere with the self-assembly of the protein into a polyhedral shape, while (2) locking the meta-stable protein in its pre-fusion conformation.

The present invention, in general, relates to nanoparticles comprising a viral fusion protein, or fragment (e.g., truncation) or variant thereof, and a self-assembling molecule, and immunogenic and vaccine compositions including the same.

Accordingly, in one aspect, the invention relates to a nanoparticle comprising a viral fusion protein, or fragment thereof, and a self-assembling molecule, wherein the self-assembling molecule forms a polymeric assembly that captures the viral fusion protein in a meta-stable pre-fusion conformation thereby forming the nanoparticle. In some embodiments, the viral fusion protein is a class I, II, or III fusion protein.

In some aspects, the invention relates to an immunogenic composition comprising a nanoparticle of the invention and a pharmaceutically acceptable carrier.

In some aspects, the invention relates to a vaccine composition comprising a nanoparticle as described herein, wherein viral fusion protein homotrimers, such as RSV F protein homotrimers (e.g., homotrimers of the ectodomain or fragments thereof, e.g., SEQ ID NOs: 1-12), in a pre-fusion conformation are displayed on the surface of a shell formed by polymeric assembly of the self-assembly molecule. In some embodiments, the vaccine composition comprises an adjuvant.

In some embodiments, the viral fusion protein adopts a dimeric or trimeric quaternary structure.

In some embodiments, the viral fusion protein is a Paramyxoviridae fusion protein. In some embodiments, the Paramyxoviridae viral fusion protein is a Paramyxoviridae or Pneumonviridae fusion protein of Avulavirus, Respirovirus, or Pneumovirus. In some embodiments, the viral fusion protein is from a virus selected from the group consisting of New Castle disease virus, Sendai virus, and Respiratory syncytial virus (RSV). In some embodiments, the virus is RSV and the fusion protein is F protein.

In some embodiments, the Flaviviridae viral fusion protein is from a Flavivirus, such as West Nile virus, Dengue virus, or yellow fever virus. In some embodiments, the virus is Dengue virus and the fusion protein is E protein.

In another aspect, the invention relates to a nanoparticle comprising Respiratory Syncytial Virus (RSV) F protein, or fragment thereof, and a self-assembling molecule, wherein the self-assembling molecule forms a polymeric assembly that captures the F protein or fragment thereof in a meta-stable pre-fusion conformation, thereby forming the nanoparticle.

In some embodiments, the F protein, or fragment thereof, lacks the transmembrane domain and cytotail domain (amino acids 1-524; SEQ ID NO: 2). In some embodiments, the F protein, or fragment thereof, lacks the transmembrane domain, cytotail domain, and a portion of the HRB domain (amino acids 1-513; SEQ ID NO: 3). In some embodiments, the F protein, or fragment thereof, comprises the F1 domain (e.g., from about amino acid 137 to about amino acid 524 (SEQ ID NO: 5), or from about amino acid 137 to about amino acid 513 (SEQ ID NO: 6)). In some embodiments, the F protein, or fragment thereof, comprises an F protein, or fragment thereof, without the N-terminal sequence MELLILKANAITTILTAVTFCFASG (SEQ ID NO: 54). In some embodiments, the F protein comprises an F1 and F2 heterodimer. In some embodiments, the F protein fragment comprises an ectodomain. In some embodiments, the F protein fragment comprises a heptad-repeat A domain (HRA) and a heptad-repeat C domain (HRC). In some embodiments, the F protein fragment comprises an HRA domain, and HRC domain, and F1 domains I and II. In some embodiments, the F protein fragment comprises an HRA domain, an HRC domain, F1 domains I and II, and a heptad-repeat B domain (HRB). In some embodiments, the nanoparticle comprises one or more homotrimers of F1 and F2.

In some embodiments, the F protein comprises an amino acid sequence set forth in SEQ ID NOs: 1-12.

In some embodiments, the viral fusion protein or RSV F protein is covalently attached to the self-assembling molecule via an amino acid linker, such as a (GlySer)n linker. In some embodiments, the linker is about 4 to 7 amino acids long. In some embodiments, the linker is of sufficient length to prevent steric hindrance between the self-assembling molecule and viral fusion protein. In some embodiments, the viral fusion protein or RSV F protein (e.g., an F protein of SEQ ID NOs: 1-12) is genetically fused to the self-assembling molecule. In one embodiment, the linker attachment point on the F protein is leucine at position 513 of SEQ ID NO: 1, and the linker attachment point on ferritin is aspartic acid at position 5 of SEQ ID NO: 13.

In some embodiments, the self-assembling molecule is proteinaceous or non-proteinaceous. In some embodiments, the self-assembling molecule is a protein, peptide, nucleic acid, a virus-like particle, a viral capsid, lipid, or carbohydrate. In some embodiments, the self-assembling molecule assembles into a shell with polyhedral symmetry, such as an octahedral symmetry. In some embodiments, the shell comprises twenty four monomers of the self-assembling molecule.

In some embodiments, the self-assembling molecule is ferritin, heat shock protein, Dsp, lumazine synthase, DNA, or those described infra. In some embodiments, the ferritin protein comprises an amino acid sequence set forth in SEQ ID NOs: 13-17. In some embodiments, the heat shock protein comprises an amino acid sequence set forth in SEQ ID NOs: 36-42.

In another aspect, the invention relates to an RSV F protein-ferritin fusion protein comprising the amino acid sequence set forth in SEQ ID NO: 18-21, 24-27, and 30-33.

In another aspect, the invention relates to an RSV F protein-heat shock protein fusion protein comprising the amino acid sequence set forth in SEQ ID NO: 22, 23, 28, 29, 34, and 35.

In some embodiments, the RSV F protein sequence is preceded by an N-terminal leader, e.g., a human CD5 leader (SEQ ID NO: 51), in order to facilitate effective secretion of the recombinant protein from transfected cells (e.g., 293 cells) into the culture medium. Exemplary leader sequence include those set forth in SEQ ID NOs: 51-53, and can be used in conjunction with any of the RSV F protein-self assembling molecule fusion proteins disclosed herein. Exemplary RSV F-ferritin fusion proteins comprising an N-terminal leader are set forth in SEQ ID NOs: 30-33. It will be understood by those of ordinary skill that the N-terminal leader can be any sequence known in the art to facilitate secretion of a protein from cells.

In another aspect, the invention relates to a method of producing an antibody which inhibits and/or prevents RSV infection comprising administering to a subject the nanoparticle, immunogenic composition, vaccine composition, or fusion protein of the present invention. In some embodiments, the antibody is isolated from the subject.

In another aspect, the invention relates to a method of producing a vaccine against RSV, the method comprising a) expressing a complex comprising a monomeric self-assembly molecule and an RSV F protein under conditions such that F protein trimers in a pre-fusion conformation are displayed on the surface of a shell formed by polymerization of the self-assembly molecule, and b) recovering the shell displaying the F protein. In some embodiments, a subject is vaccinated with the vaccine of the present invention.

In another aspect, the invention relates to a kit comprising the nanoparticle or fusion protein of the invention and instructions for use.

In yet other aspects, the invention relates to isolated nucleic acids encoding the nanoparticles or fusion proteins of the invention, vectors comprising the nucleic acids, and isolated cells comprising the nucleic acids.

### II. Meta-Stable Protein-Self Assembling Molecule Fusions

The nanoparticles of the present invention comprise a meta-stable protein fused to a proteinaceous or non-proteinaceous self-assembling molecule. The present invention can be applied to any meta-stable protein (e.g., a meta-stable viral fusion protein) for which it is beneficial to retain the meta-stable protein in the higher energy, less stable, conformation, i.e., preventing the meta-stable protein from adopting the lower energy stable conformation. In the context of one exemplary meta-stable viral protein, the RSV F protein, the F protein is locked into a higher energy pre-fusion state upon formation of the F protein trimer and polymeric assembly of the self-assembling molecule to which it is fused. Accordingly, the nanoparticle surface displays the trimeric F protein in the pre-fusion state, for example, as assessed by an antibody which specifically recognizes the pre-fusion state (e.g., mAb D25), as described in Example 2.

The F glycoprotein of RSV directs viral penetration by fusion between the virion envelope and the host cell plasma membrane. It is a type I single-pass integral membrane protein having four general domains: N-terminal ER-translocating signal sequence (SS), ectodomain (ED), transmembrane domain (TM), and a cytoplasmic tail (CT). The cytoplasmic tail contains a single palmitoylated cysteine residue. Although the sequence of the F protein is highly conserved among RSV isolates, it is constantly evolving. The F protein in RSV differs from the F protein of other paramyxoviruses because it can mediate entry and syncytium formation independent of the other viral proteins (HN is usually necessary in addition to F in other paramyxoviruses).

The RSV F mRNA is translated into a 574 amino acid precursor protein designated F₀, which contains a signal peptide sequence at the N-terminus that is removed by a signal peptidase in the endoplasmic reticulum. F₀ is cleaved at two sites (aa 109/110 and 136/137) by cellular proteases (e.g., furin) in the trans-Golgi, removing a short glycosylated intervening sequence and generating two subunits designated F₁ (about 50 kDa; C-terminus; residues 137-574) and F₂ (about 20 kDa; N-terminus; residues 1-109). F₁ contains a hydrophobic fusion peptide at its N-terminus and also two hydrophobic heptad-repeat regions (HRA and HRB). HRA is near the fusion peptide and HRB is near to the transmembrane domain. The F₁-F₂ heterodimers are assembled as homotrimers in the virion.

RSV exists as a single serotype but has two antigenic subgroups: A and B. The F glycoproteins of the two groups are about 90% identical. The A subgroup, the B subgroup, or a combination or hybrid of both can be used in the invention. An example sequence for the A subgroup is SEQ ID NO: 1 (A2 strain; GenBank GI: 138251; Swiss Prot P03420), and for the B subgroup is SEQ ID NO: 7 (18537 strain; GI: 138250; Swiss Prot P13843). SEQ ID NO:1 and SEQ ID NO:7 are both 574 amino acid sequences. The signal peptide in the A2 strain is aa 1-21, but in the 18537 strain it is 1-22. In both sequences the TM domain is from about aa 530-550, but has alternatively been reported as 525-548. Either the A and/or B subgroups are suitable for use in the nanoparticles of the present invention. Also suitable for use in the nanoparticles described herein are fragments of the F protein from A2 and 18537 strains (e.g., SEQ ID NOs: 1 and 7, respectively).

The RSV F protein is known to exist in three conformations: the pre-fusion, post-fusion, and intermediate-fusion conformations. The “post-fusion conformation” of RSV F protein is believed to be a low energy conformation of native RSV F, and is a trimer characterized by the presence of a six-helix bundle comprising 3 HRB and 3HRA regions. The post-fusion conformation has a characteristic “crutch” or “golf tee” shape by electron microscopy. The “pre-fusion conformation” of RSV F protein is a meta-stable conformation characterized by a trimer that contains a coiled coil comprising 3 HRB regions. The fusion peptide is not exposed in the pre-fusion conformation and, therefore, pre-fusion conformations generally do not form rosettes, and have a “lollipop” or “ball and stem” shape by electron microscopy.

As discussed supra, the present invention relates to locking the RSV F protein, or a fragment thereof, in the higher energy “pre-fusion,” or “meta-stable,” conformation by fusion to a self-assembling molecule such that the trimeric F protein is locked in the pre-fusion conformation within the polymeric assembly of the self-assembling molecule; (see Example 1). For example, the F-glycoprotein of RSV adopts a trimeric quaternary structure in its pre-fusion conformation. The epitopes of the pre-fusion conformation may be better able to elicit antibodies that can recognize and neutralize natural virions. Without wishing to be bound by any particular theory, it is believed that the pre-fusion conformation may contain epitopes similar or identical to those expressed on natural RSV virions, and therefore provide advantages for eliciting neutralizing antibodies. An epitope specific to the pre-fusion conformation F protein is an epitope that is not presented in the intermediate-fusion or post-fusion conformation. It is preferred that the at least one epitope of the pre-fusion conformation F protein is stably presented, e.g., the epitope is stably presented in solution for at least twelve hours, at least one day, at least two days, at least four days, at least six days, at least one week, at least two weeks, at least four weeks, or at least six weeks.

The basic unit of the nanoparticles of the present invention is the meta-stable protein monomer fused to a self-assembling molecule monomer via a linker (e.g., a monomeric RSV F protein-linker-ferritin monomer polypeptide). For simplicity, the RSV F protein-linker-ferritin fusion protein is an exemplary meta-stable protein-self-assembling molecule fusion protein. The precise points of attachment of the linker (as well as the optimal length of the linker) to the meta-stable viral fusion protein (e.g., RSV F protein) and self-assembling molecule (e.g., ferritin) can be determined computationally, as described in Example 1, and ensures that the viral fusion protein is able to adopt its native pre-fusion conformation when assembled into trimeric form, while not interfering with the polymeric assembly of the self-assembling molecule. With respect to the RSV F protein and ferritin, the next structural level is the trimeric assembly of the RSV F protein and trimeric assembly of ferritin. In this embodiment, the fusion protein is designed such that neither assembly of the RSV F protein in its pre-fusion conformation nor polymeric assembly of the ferritin is hindered. Trimeric ferritin then assembles into a shell with octahedral symmetry, thereby forming the nanoparticle, wherein eight ferritin trimers and RSV F protein trimers are aligned, and wherein the RSV F protein trimers are stably displayed on the surface in the pre-fusion conformation.

As would be understood by one of ordinary skill, the present invention can be applied to other viral proteins sharing similar features with the RSV F protein, i.e., those having both a pre- and post-fusion conformations. In some embodiments, the viral fusion protein is a class I fusion protein (e.g., paramyxovirus F protein, influenza HA). In other embodiments, the viral fusion protein is a class II fusion protein (e.g., TBEV E protein, SFV E1/E2). In yet other embodiments, the viral fusion protein is a class III fusion protein (e.g., VSV G). Class I-III viral fusion proteins are described extensively in detail in the literature (see, e.g., Colman and Lawrence. Nature Reviews Molecular Cell Biology 2003; 4:309-19; White et al., Crit Rev Biochem Mol Biol 2008; 43:189-219) incorporated herein by reference. In other embodiments, the fusion protein is a class I viral fusion protein with the exception of influenza HA protein. Further exemplary viral proteins exhibiting this characteristic include, but are not limited to, pneumoviridae, paramyxoviridae, flaviviridae, and retroviridae viral fusion proteins. Accordingly, in some embodiments, the viral fusion protein is that of New Castle disease virus, Sendai virus, flavivirus (e.g., West Nile virus, Dengue virus, yellow fever virus, and the like), and human immunodeficiency virus (HIV). In one embodiment, the virus is Dengue virus and the fusion protein is E protein.

The meta-stable protein for use in the invention can be fused to a self-assembling molecule known in the art. As described in further detail infra, the self-assembling molecule can be proteinaceous or non-proteinaceous. In a preferred embodiment, the self-assembling molecule is proteinaceous. In some embodiments, the self-assembling molecule assembles into a shell with polyhedral symmetry. In one embodiment, the shell has an octahedral symmetry. In yet another embodiment, the shell comprises 24 monomers of the self-assembling molecule. In some embodiments, the self-assembling molecule is ferritin (which has octahedral symmetry and forms a shell with 24 monomers). In other embodiments, the self-assembling molecule is a heat shock protein (HSP), e.g., small HSP20 (sHSP20: SEQ ID NOs: 36 or 37), HSP100 (SEQ ID NO: 42), HSP90 (SEQ ID NOs: 40 or 41), HSP70 (SEQ ID NO: 39), and HSP60 (SEQ ID NO: 38), or a fragment thereof.

In some embodiments, the meta-stable protein is chemically fused to the self-assembling molecule. In preferred embodiments, the meta-stable protein is genetically fused to the self-assembling molecule, with or without a linker, such as a polypeptide linker. A linker sequence can be inserted so that the RSV F protein is positioned in such a way to maintain the ability to elicit an immune response to RSV. In one embodiment, the meta-stable RSV F protein is linked to ferritin in a manner that will preserve the proper orientation and/or lock the meta-stable viral protein in its pre-fusion conformation. For example, the meta-stable viral protein may be attached to the self-assembling protein (e.g., ferritin) such that it does not hinder the self-assembly of the protein into a polyhedral shape. For example, to orient the RSV F protein trimers and ferritin trimers about the same three-fold axis, the Leu-513 residue of the RSV F protein may be linked to the Asp-5 residue of ferritin. In one embodiment, the Leu-513 residue of the RSV F protein is linked to the Asp-5 residue of ferritin through the use of a 4-7 amino acid linker. The linker may comprise a mixture of glycine and serine residues (a ser-gly linker).

Linker sequences of the present invention comprise amino acids. Preferable amino acids to use are those having small side chains and/or those which are not charged. Such amino acids are less likely to interfere with proper folding and activity of the fusion protein. Accordingly, preferred amino acids to use in linker sequences, either alone or in combination are serine, glycine and alanine. The composition of the linker may be alternating serine and glycine residues. Alternating serine and glycine residues may allow for flexibility in the conformation. Examples of linker sequences include, but are not limited to, SGG, GSG, SGS, GG, SGSG (SEQ ID NO: 43), NGTGGSG (SEQ ID NO: 44), SGGSG (SEQ ID NO: 45), GGSGSG (SEQ ID NO: 46), SGSGSG (SEQ ID NO: 47), SGGSGSG (SEQ ID NO: 48), SGSGSGSGS (SEQ ID NO: 49) and SGSGSGSGSG (SEQ ID NO: 50). Amino acids can be added or subtracted as needed. For instance, the linker may be 1, 2, 3, 4, 5, 6, 7, 8, 9, 10, 11, 12, 15, 20 or more amino acids in length. For example, the linker may be 4-7, 5-8, 5-9, 5-10, 6-10, 7-10, 8-10, or 9-10 amino acids in length. Those skilled in the art are capable of determining appropriate linker sequences, as well as the appropriate linker length, for fusion proteins of the present invention. For example, the approach described in Example 1 can be used to design suitable linkers to separate the meta-stable protein from the self-assembling molecule.

In some embodiments, the nanoparticles of the present invention include a meta-stable protein variant and/or self-assembling molecule variant and/or meta-stable protein-self-assembling molecule fusion variant. For example, the meta-stable protein variant and/or a self-assembling molecule variant and/or meta-stable protein-self-assembling molecule fusion variant can be 50%, 60%, 70%, 80%, 85%, 90%, 95%, 98%, or 100% identical to the corresponding wild-type meta-stable protein and/or wild-type self-assembling molecule and/or wild-type meta-stable protein-self-assembling molecule fusion. Accordingly, in some embodiments, the present invention relates to a nanoparticle which includes an RSV F protein variant and/or self-assembling molecule variant and/or RSV F protein-self-assembling molecule fusion protein variant that is at least about 50%, at least about 60%, at least about 70%, at least about 80%, at least about 85%, at least about 90%, at least about 95%, at least about 98%, at least about 99%, or 100% identical to the wild-type RSV F protein and/or wild-type self-assembling molecule and/or wild-type RSV F protein-self-assembling molecule fusion protein. Thus, in some embodiments, the present invention provides polypeptides that are at least about 50%, at least about 60%, at least about 70%, at least about 80%, at least about 85%, at least about 90%, at least about 95%, at least about 98%, at least about 99% identical to any one of SEQ ID NOs: 1-42. In some embodiments, the meta-stable protein variant and/or a self-assembling molecule variant and/or meta-stable protein-self-assembling molecule fusion variant can have at least one mutation (e.g., deletion, addition, or substitution of 1, 2, 3, 4, 5, 6, 7, 8, 9, 10, 11, 12, 13, 14, 15, 16, 17, 18, 19, 20, or more amino acid residues). Accordingly, in some embodiments, the present invention relates to a nanoparticle which includes an RSV F protein variant and/or self-assembling molecule variant and/or RSV F protein-self-assembling molecule fusion protein variant having at least one mutation (e.g., deletion, addition, or substitution of 1, 2, 3, 4, 5, 6, 7, 8, 9, 10, 11, 12, 13, 14, 15, 16, 17, 18, 19, 20, or more amino acid residues) relative to the wild type RSV F protein and/or wild-type self-assembling molecule and/or wild-type RSV F protein-self-assembling molecule fusion protein. Preferably, the mutations do not alter the pre-fusion conformation of the trimeric RSV F protein or the self-assembling ability of the self-assembling molecule. Various assays are available, as described infra, to assess whether nanoparticles comprising the variants retain the ability to elicit the production of RSV neutralizing antibodies that target the F protein in the pre-fusion conformation.

In the variants described above which have amino acid substitutions, the substituted amino acid residue(s) can be, but are not necessarily, conservative substitutions, which typically include substitutions within the following groups: glycine, alanine; valine, isoleucine, leucine; aspartic acid, glutamic acid; asparagines, glutamine; serine, threonine; lysine, arginine; and phenylalanine, tyrosine.

In some embodiments, a fragment of the meta-stable protein may be used in place of the entire meta-stable protein sequence, provided that an epitope of the meta-stable protein that elicits the production of an antibody that specifically recognizes the meta-stable protein conformation is available and displayed. In the context of RSV F protein, in one embodiment, a fragment of the RSV F protein can be fused to a self-assembly molecule, e.g., via a linker, provided that, when in the form of a nanoparticle, a pre-fusion F protein conformational epitope is displayed and can elicit the production of antibodies against the epitope. Accordingly, in some embodiments, the RSV F protein fragment can comprise at least 25 amino acids, at least 50 amino acids, at least 75 amino acids, at least 100 amino acids, at least 150 amino acids, at least 200 amino acids, at least 300 amino acids, at least 400 amino acids, or at least 500 amino acids from an RSV F protein, wherein the RSV F protein fragment elicits the production of neutralizing antibodies against the RSV F protein in the pre-fusion conformation.

In some embodiments, the RSV F fragment comprises one or more domains of the RSV F protein, such as an ectodomain, a heptad-repeat A domain (HRA) and a heptad-repeat C domain (HRC); an HRA domain, an HRC domain, and F1 domains I and II; or an HRA domain, an HRC domain, F1 domains I and II, and a heptad-repeat B domain (HRB). As discussed infra, various assays are available to one of ordinary skill to determine whether a candidate nanoparticle comprising an RSV F protein fragment-self-assembling molecule fusion elicits neutralizing antibodies that specifically recognize the pre-fusion conformation of the RSV F protein.

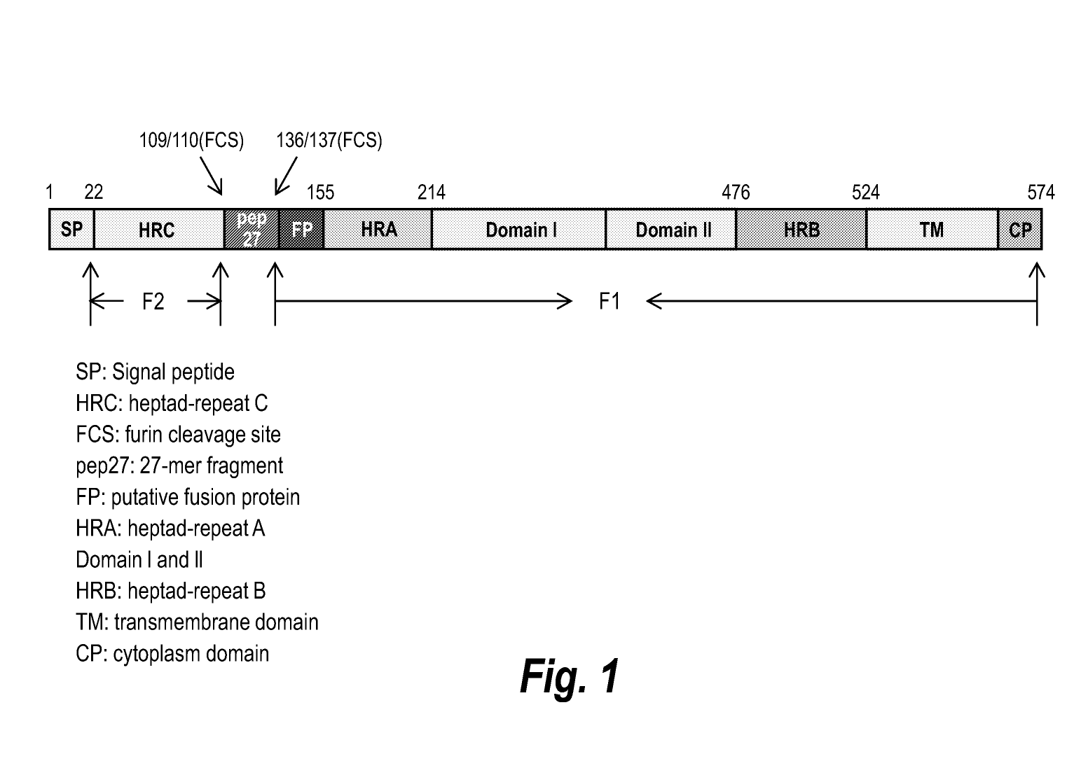

For example, the RSV F fragment may comprise one or more domains of the RSV F protein (FIG. 1), such as an ectodomain (e.g., as disclosed in US 2011/0305727, the contents of which are incorporated herein by reference) a heptad-repeat A domain (HRA) and a heptad-repeat C domain (HRC) (amino acids 22-214 of SEQ ID NO:1); an HRA domain, an HRC domain, and F1 domains I and II (amino acids 22-476 of SEQ ID NO:1); or an HRA domain, an HRC domain, F1 domains I and II, and a heptad-repeat B domain (HRB) (amino acids 22-524 of SEQ ID NO: 1). One of ordinary skill in the art can readily determine the domains of other RSV F proteins or antigenic subgroups.

In general, the fusion proteins used in the practice of the instant invention will be synthetic, or produced by expression of a recombinant nucleic acid molecule. In the event the polypeptide is an RSV F protein-ferritin fusion or RSV F protein-HSP fusion, it can be encoded by a hybrid nucleic acid molecule containing one sequence that encodes RSV F protein and a second sequence that encodes all or part of ferritin or HSP. In preferred embodiments, a linker will separate the first and second sequences.

The techniques required to make the meta-stable protein-self-assembling molecule fusion, or a variant thereof, are routine in the art, and can be performed without resort to undue experimentation. For example, a mutation that consists of a substitution of one or more of the amino acid residues in the meta-stable protein (e.g., RSV F protein) can be created using a PCR-assisted mutagenesis technique (as known in the art). Mutations that consist of deletions or additions of amino acid residues to a meta-stable protein polypeptide and/or a self assembling molecule polypeptide and/or a meta-stable protein-self-assembling molecule fusion polypeptide can also be made with standard recombinant techniques. In the event of a deletion or addition, the nucleic acid molecule encoding the meta-stable protein is simply digested with an appropriate restriction endonuclease. The resulting fragment can either be expressed directly or manipulated further by, for example, ligating it to a second fragment. The ligation may be facilitated if the two ends of the nucleic acid molecules contain complementary nucleotides that overlap one another, but blunt-ended fragments can also be ligated. PCR-generated nucleic acids can also be used to generate various mutant sequences.

In addition to generating meta-stable protein variant-self-assembling molecule fusions via expression of nucleic acid molecules that have been altered by recombinant molecular biological techniques, they can also be chemically synthesized. Chemically synthesized polypeptides are routinely generated by those of skill in the art.

As noted above, the meta-stable protein (e.g., RSV F protein) can be prepared as fusion or chimeric polypeptides that include the meta-stable protein and a self-assembling molecule. In some embodiments, the chimeric polypeptide can include the meta-stable protein, self-assembling molecule, and a polypeptide that functions as an antigenic tag, such as a FLAG sequence. FLAG sequences are recognized by biotinylated, highly specific, anti-FLAG antibodies, as described herein (see also Blanar et al., Science 256:1014, 1992; LeClair et al., PNAS 89:8145, 1992). Methods for adding antigenic tags and constructing chimeric polypeptides are well known and can be performed with conventional molecular biological techniques, which are well within the ability of those of ordinary skill in the art to perform.

### III. Self-Assembling Molecules

The fusions and fusion proteins of the present invention comprise a meta-stable viral protein (e.g., RSV F protein) and a self-assembling molecule. In some embodiments, the self-assembling molecule is a protein or peptide based molecule (e.g., ferritin, heat shock protein, DNA-binding proteins, and viral capsid proteins, or variants thereof). In other embodiments, the self-assembling molecule is not a protein or peptide-based molecule (e.g., nucleic acid, lipid, liposomes, dextran, polysaccharides, metal, etc). Any art-recognized molecule, whether proteinaceous or not, is suitable for use in the present invention, provided that the molecule self-assembles into a polyhedral symmetry which allows for the meta-stable protein to which it is fused to be stabilized and locked in its pre-fusion conformation. Moreover, it is known in the art that some variation in a protein sequence can be tolerated without significantly affecting the activity of the protein.

Thus, in one embodiment, fragments of self-assembling molecules are contemplated, so long as they retain the ability to undergo polymeric assembly and are able to present the meta-stable protein in the higher energy conformation, e.g., the pre-fusion conformation of the RSV F protein. In the context of a ferritin protein, for example, portions, or regions, of the monomeric ferritin subunit protein can be utilized so long as the portion comprises an amino acid sequence that directs self-assembly of monomeric ferritin subunits into the globular form of the protein. One example of such a region is located between amino acids 5 and 167 of the Helicobacter pylori ferritin protein. More specific regions are described in Zhang, Y. Self-Assembly in the Ferritin Nano-Cage Protein Super Family. 2011, Int. J. Mol. Sci., 12, 5406-5421, which is incorporated herein by reference in its entirety. Moreover, given the structural considerations that go into designing a suitable nanoparticle of the invention, as described in Example 1, use of a ferritin fragment may be preferred. For example, in the RSV F protein-ferritin fusion proteins described in Example 1 (i.e., SEQ ID NOs: 18-21, 24-27, and 30-33) comprise an RSV F protein fused via a linker to a ferritin fragment spanning amino acids 5-167 of Helicobacer pylori ferritin. That is, the linker is linked to the Asp-5 residue of the Helicobacer pylori ferritin. Similarly, with respect to the RSV F protein-sHSP20 fusion protein described in Example 2 (i.e., SEQ ID NOs: 22, 23, 28, 29, 34, and 35), the RSV F protein is fused via a linker to an sHSP20 fragment spanning amino acids 24-147 of sHSP20. That is, the linker is linked to the Thr-24 residue of sHSP20.

In some embodiments, the self-assembling molecules serve as delivery vehicles for various therapeutics, adjuvants, imaging agents, or molecules. For example, the various therapeutics, adjuvants, imaging agents, or molecules can be loaded into the interior space of the self-assembled molecule, or may be attached to the self-assembled molecule.

### Ferritin

Ferritin is a globular protein found in all animals, bacteria, and plants, that acts primarily to control the rate and location of polynuclear Fe(III)₂O₃ formation through the transportation of hydrated iron ions and protons to and from a mineralized core. The globular form of ferritin is made up of monomeric subunit proteins (also referred to as monomeric ferritin subunits), which are polypeptides having a molecule weight of approximately 17-20 kDa. An example of the sequence of one such monomeric ferritin subunit is represented by SEQ ID NO: 13. Each monomeric ferritin subunit has the topology of a helix bundle which includes a four antiparallel helix motif, with a fifth shorter helix (the c-terminal helix) lying roughly perpendicular to the long axis of the 4 helix bundle. According to convention, the helices are labeled “A, B, C, and D & E” from the N-terminus respectively. The N-terminal sequence lies adjacent to the capsid threefold axis and extends to the surface, while the E helices pack together at the four-fold axis with the C-terminus extending into the particle core. The consequence of this packing creates two pores on the capsid surface. It is expected that one or both of these pores represent the point by which the hydrated iron diffuses into and out of the capsid. Following production, these monomeric ferritin subunit proteins self-assemble into the globular ferritin protein (i.e., a shell with polyhedral symmetry). Thus, the globular form of ferritin (i.e., ferritin shell) comprises twenty-four monomeric, ferritin subunit proteins, and has a capsid-like structure having 432 symmetry (i.e., octahedral symmetry). The twenty-four ferritin monomers occupy each of the twenty-four symmetry domains.

A monomeric ferritin subunit of the present invention is a full length, single polypeptide of a ferritin protein, or any portion thereof, which is capable of directing self-assembly of monomeric ferritin subunits into the globular form of the protein. Amino acid sequences from monomeric ferritin subunits of any known ferritin protein can be used to produce fusion proteins of the present invention, so long as the monomeric ferritin subunit is capable of self-assembling into a nanoparticle displaying RSV F on its surface in the pre-fusion conformation. Whether the F protein is expressed in the pre-fusion conformation can be determined using, e.g., an antibody that specifically recognizes the pre-fusion conformation (e.g., the D25 mAb), as described, e.g., in Example 2. In one embodiment, the monomeric subunit is from a ferritin protein selected from the group consisting of a bacterial ferritin protein, a plant ferritin protein, an algal ferritin protein, an insect ferritin protein, a fungal ferritin protein and a mammalian ferritin protein. In one embodiment, the ferritin protein is from Helicobacter pylori. In another embodiment, the ferritin protein is from Homo sapiens.

### Heat Shock Proteins

Heat shock proteins (HSPs) are known to self-assemble with a polyhedral symmetry. Suitable heat shock proteins for use in the present invention include HspG41C (see, e.g., Kaiser et al. Int J Nanomedicine 2007; 2:715-33) and the sHSP homologue of Methanococcus jannaschii, which forms a homogeneous multimer of 24 monomers with octahedral symmetry (Kim et al., PNAS 1998; 95:9129-33; Kim et al., Nature 1998; 394:595-9; US2007/0258889; Flenniken et al., Nano Lett 2003; 3:1573-6). Additional heat shock proteins that are suitable for use in the invention include, but are not limited to, HSP60, HSP70, HSP90, and HSP100, or fragments thereof which retain the ability to self-assemble.

In a preferred embodiment, the meta-stable protein of the invention (e.g., RSV F protein) is fused to sHSP (SEQ ID NOs: 36 or 37). In one embodiment, the RSV F protein-sHSP fusion has the sequence set forth in SEQ ID NOs: 22, 23, 28, 29, 34, or 35. Other suitable HSPs for use as self-assembling molecules include, but are not limited to, HSP60 (SEQ ID NO: 38), HSP70 (SEQ ID NO: 39), HSP90 (SEQ ID NOs: 40-41), and HSP100 (SEQ ID NO: 42).

### Viruses

Given their highly organized repeating motifs and symmetry, viruses also present suitable cages for use in the present invention. Suitable, but non-limiting, viruses include Cowpea chlorotic mottle virus (CCMV) (Speir et al., Structure 1995; 3:63-78; Gillitzer et al., Chem Common (Camb) 2002; 21:2390-1, Gillitzer et al., Small 2006; 2:962-6; Brumfield et al., J Gen Virol 2004; 85:1049-53; US2007/0258889); Cowpea mosaic virus (CPMV) (Brennan et al., Mol Biotechnol 2001; 17:15-26; Chatterji et al., Intervirology 2002; 45:362-70); Raja et al., Biomacromolecules 2003; 4:472-6; Blum et al., Nano Letters 2004; 4:867-70; Rae et al., Virology 2005; 343:224-35; Lewis et al., Nat Med 2006; 12:354-60), potato virus X (PVX; Marusic et al., J Virol 2001; 75:8434-9); MS2 virus (US2007/0258889); and tobacco mosaic virus (Koo et al. PNAS 1999; 96:7774-9; Smith et al. Virology 2006; 348:475-88).

### Dps and Dps-Like Proteins

Also suitable for use as protein cages in the present invention are Dps and Dps-like proteins, such as those from E. coli (Almiron et al. Genes Dev 1992; 6:2646-54; Ilari et al. JBC 2002:277: 27619-623), Helicobacter pylori (Tonello et al. Mol Microbiol 1999; 34:238-46), Halobacterium salinarum (Zeth et al. PNAS 2004; 101:13780-5), Bacillus anthracis (Papinuttto et al. PNAS 2002; 277:15093-8), Sulfolobus solfataricus, Pyrococcus furiosus, and Listeria innocua (Iari et al. Acta crystallogr 1999; D55:552-3; Stefanini et al. Biochem J 1999; 338:71-75; Bozzi et al. JBC 1997; 272:3259-265; Su et al. Biochemistry 2005; 44:5572-8).

### Others

Other art-recognized self-assembling molecules include lumazine synthase (Shenton et al. Angewandte Chemie-International Edition 2001; 40:442-5); liposomes (Lee and Low Biochim biophys Acta 1995; 1233:134-44; Muller et al. Cancer Gene Ther 2001; 8:107-17; Barratt et al. Cell Mol Life Sci 2003; 60:21-37); micelles (Roy et al. J Am Chem Soc 2003; 125:7860-5; polyamidoamine dendrimer clusters (Choi et al. Chem Biol 2005; 12:35-43; Gurdag et al. Bioconjug Chem 2006; 17:375-83); poly (D, L-lactic-co-glycolic acid nanoparticles (Yoo et al. Pharm Res 1999; 16:1114-8; Yoo et al. J Control Release 2000; 68:419-31); hydrogel dextran nanoparticles (Jana et al. FEBS Lett 2002; 515:184-8; Na and Bae. Pharm Res 2002; 19:681-8); polysaccharide nanoparticles (Janes et al. Adv Drug Deliv Rev 2001; 47:83-97); polyalkylcyanoacrylate nanocapsules (Damge et al. Diabetes 1998; 87:246-51); lipid nanoparticles (Fundaro et al. Pharmacol Res 2000; 42:337-43); metal nanoshells (Loo et al. Opt Lett 2005; 30:1012-4; Loo et al. Technol Cancer Res Treat 2004; 3:33-40); amphiphilic core-shell nanoparticles (Sun et al. Biomacromolecules 2005; 6:2541-54); other protein cage-based nanostructures (Hooker et al. J Am Chem Soc 2004; 126:3718-9); silica nanoparticles, and albumin.

### IV. Nucleic Acid Molecules Encoding Meta-Stable Protein-Self-Assembling Molecule Fusion Proteins

The present invention also relates to nucleic acids which encode the meta-stable protein-self-assembling molecule fusion proteins of the invention. The meta-stable protein-self-assembling molecule fusion proteins, such as those described above, can be obtained by expression of a nucleic acid molecule. Thus, nucleic acid molecules encoding polypeptides containing a meta-stable protein-self-assembling molecule fusion are considered within the scope of the invention. Just as meta-stable protein variants-self-assembling molecule fusion proteins can be described in terms of their identity with a wild-type meta-stable protein-self-assembling molecule fusion protein (i.e., the meta-stable protein and/or self-assembling molecule is wild-type), the nucleic acid molecules encoding them will necessarily have a certain identity with those that encode the wild-type meta-stable protein (e.g., RSV F protein) and/or self-assembling molecule (e.g., ferritin, HSP). For example, the nucleic acid molecule encoding an meta-stable protein variant and/or self-assembling molecule variant and/or meta-stable protein-self-assembling molecule fusion variant can be at least 50%, at least 65%, preferably at least 75%, more preferably at least 85%, and most preferably at least 95% (e.g., 99%) identical to the nucleic acid encoding wild-type meta-stable protein and/or wild-type self-assembling molecule and/or meta-stable protein-self-assembling molecule fusion variant, respectively.

The nucleic acid molecules of the invention can contain naturally occurring sequences, or sequences that differ from those that occur naturally, but, due to the degeneracy of the genetic code, encode the same polypeptide. These nucleic acid molecules can consist of RNA or DNA (for example, genomic DNA, cDNA, or synthetic DNA, such as that produced by phosphoramidite-based synthesis), or combinations or modifications of the nucleotides within these types of nucleic acids. In addition, the nucleic acid molecules can be double-stranded or single-stranded (i.e., either a sense or an antisense strand).

The nucleic acid molecules are not limited to sequences that encode polypeptides; some or all of the non-coding sequences that lie upstream or downstream from a coding sequence can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

The isolated nucleic acid molecules of the invention can include fragments not found as such in the natural state. Thus, the invention encompasses recombinant molecules, such as those in which a nucleic acid sequence is incorporated into a vector (e.g., a plasmid or viral vector) or into the genome of a heterologous cell (or the genome of a homologous cell, at a position other than the natural chromosomal location).

### V. Methods of Expression/Methods of Making

The nucleic acid molecules described above can be contained within a vector that is capable of directing their expression in, for example, a cell that has been transduced with the vector. Accordingly, expression vectors containing a nucleic acid molecule encoding a meta-stable protein-self-assembling molecule fusion protein (e.g., RSV F protein-ferritin fusion, RSV F protein-HSP fusion) and cells transfected with these vectors are among the preferred embodiments.

Vectors suitable for use in the present invention include T7-based vectors for use in bacteria (see, for example, Rosenberg et al., Gene 56:125, 1987), the pMSXND expression vector for use in mammalian cells (Lee and Nathans, J. Biol. Chem. 263:3521, 1988), and baculovirus-derived vectors (for example the expression vector pBacPAK9 from Clontech, Palo Alto, Calif.) for use in insect cells. The nucleic acid inserts, which encode the polypeptide of interest in such vectors, can be operably linked to a promoter, which is selected based on, for example, the cell type in which expression is sought. For example, a T7 promoter can be used in bacteria, a polyhedrin promoter can be used in insect cells, and a cytomegalovirus or metallothionein promoter can be used in mammalian cells. Also, in the case of higher eukaryotes, tissue-specific and cell type-specific promoters are widely available. These promoters are so named for their ability to direct expression of a nucleic acid molecule in a given tissue or cell type within the body. Skilled artisans are well aware of numerous promoters and other regulatory elements which can be used to direct expression of nucleic acids.

In addition to sequences that facilitate transcription of the inserted nucleic acid molecule, vectors can contain origins of replication, and other genes that encode a selectable marker. For example, the neomycin-resistance (neor) gene imparts G418 resistance to cells in which it is expressed, and thus permits phenotypic selection of the transfected cells. Those of skill in the art can readily determine whether a given regulatory element or selectable marker is suitable for use in a particular experimental context.

Viral vectors that can be used in the invention include, for example, retroviral, adenoviral, and adeno-associated vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

Prokaryotic or eukaryotic cells that contain and express a nucleic acid molecule that encodes a meta-stable protein-self-assembling molecule fusion protein (e.g., RSV F protein-ferritin fusion, RSV F protein-HSP fusion), or a variant thereof, are also features of the invention. A cell of the invention is a transfected cell, i.e., a cell into which a nucleic acid molecule, for example a nucleic acid molecule encoding meta-stable protein-self-assembling molecule fusion protein (e.g., RSV F protein-ferritin fusion, RSV F protein-HSP fusion), or a variant thereof, has been introduced by means of recombinant DNA techniques. The progeny of such a cell are also considered within the scope of the invention.

The precise components of the expression system are not critical. For example, a meta-stable protein-self-assembling molecule fusion protein (e.g., RSV F protein-ferritin fusion, RSV F protein-HSP fusion), or variant thereof, can be produced in a prokaryotic host, such as the bacterium E. coli, or in a eukaryotic host, such as an insect cell (e.g., an Sf21 cell), or mammalian cells (e.g., COS cells, NIH 3T3 cells, or HeLa cells). These cells are available from many sources, including the American Type Culture Collection (Manassas, Va.). In selecting an expression system, it matters only that the components are compatible with one another. Artisans or ordinary skill are able to make such a determination. Furthermore, if guidance is required in selecting an expression system, skilled artisans may consult Ausubel et al. (Current Protocols in Molecular Biology, John Wiley and Sons, New York, N.Y., 1993) and Pouwels et al. (Cloning Vectors: A Laboratory Manual, 1985 Suppl. 1987).

The expressed polypeptides can be purified from the expression system using routine biochemical procedures, and can be used, e.g., as therapeutic/prophylactic agents (e.g., vaccines), as described herein.

In some aspects, the meta-stable protein-self-assembling molecule fusion proteins can be made by synthetic methods. For example, solid phase synthesis techniques may be used. Suitable techniques are well known in the art, and include those described in Merrifield (1973), Chem. Polypeptides, pp. 335-61 (Katsoyannis and Panayotis eds.); Merrifield (1963), J. Am. Chem. Soc. 85: 2149; Davis et al. (1985), Biochem. Intl. 10: 394-414; Stewart and Young (1969), Solid Phase Peptide Synthesis; U.S. Pat. No. 3,941,763; Finn et al. (1976), The Proteins (3rd ed.) 2: 105-253; and Erickson et al. (1976), The Proteins (3rd ed.) 2: 257-527.

Other methods of molecule expression/synthesis are generally known in the art to one of ordinary skill.

### VI. In Vivo Assays/Models

The invention provides methods for assaying the immunogenicity of the candidate nanoparticle comprising the RSV F-ferritin fusion-based vaccines described herein in a subject. In one embodiment, the subject may be a primate (e.g., humans, chimpanzees, monkeys, baboons), rats (e.g., cotton rats), mice, calves, guinea pigs, ferrets and hamsters. In some embodiments, the subject may be immunocompromised.

The subject may be vaccinated intramuscularly with various doses of the fusion protein. The subject may be vaccinated one, two, three, four or more times with the fusion protein. For example, the subject may be vaccinated on day 0 and day 21. Alternatively, the subject may be vaccinated on day, 0, day 14 and day 28, or the subject may be vaccinated on day 0, day 21 and day 35, or the subject may be vaccinated on day 0, day 14, day 28 and day 42. The subject may also be vaccinated with a later booster shot.

The serum anti-RSV F antibody titers may be measured after each vaccination and control serum titers may also be measured prior to immunization. In some embodiments, serum anti-RSV F antibody titers are measured two or three weeks after the first vaccination and two or three weeks after the second vaccination. In other embodiments, serum anti-RSV F antibody titers are measured two or three weeks after the second, third and/or fourth vaccination. Antibody titers may be determined by methods known in the art. For example, antibody titers may be assayed by ELISA, Immunoblot assays or indirect immunofluorescence. The antigen used in these assays may be an RSV-F protein, such as a trimeric RSV F protein in the pre-fusion conformation. Specificity of the generated antibodies for the RSV F protein pre-fusion conformation over the RSV F post-fusion conformation can be determined by, e.g., ELISA assays which use trimeric RSV F protein in the pre-fusion conformation or the post-fusion conformation as the antigen. A candidate nanoparticle vaccine can be considered a pre-fusion trimeric F protein targeting antibody inducing vaccine if the antibodies isolated from the vaccinated subject binds with a greater affinity to the trimeric RSV F protein in the pre-fusion conformation compared to trimeric RSV F protein in the post-fusion conformation by at least 1.5-fold, such as at least 2-fold, at least 2.5-fold, at least 3-fold, at least 3.5-fold, at least 4-fold, at least 4.5-fold, at least 5-fold or greater.

In some embodiments, the vaccinated subjects may be challenged intranasally with RSV after the final immunization to determine whether neutralizing antibodies were generated in the vaccinated subject. For example, the subjects may be challenged with RSV three, four or five weeks after the final immunization. In some embodiments, control unvaccinated subjects are challenged with RSV concurrently with the vaccinated subjects.

Serum samples from vaccinated subjects may be tested for the presence of neutralizing antibodies by microneutralization assay. Microneutralization assays may be performed by methods known in the art. The number of infectious virus particles may be determined by detection of syncytia formation by immunostaining. The neutralization titer may be defined as the reciprocal of the serum dilution producing at least a 60% reduction in number of synctia per well, relative to controls (no serum).

Viral load in the lung of the subjects may be determined by plaque assay. The lungs of the subjects may be harvested post RSV infection and a plaque assay may be used to test for infectious virus. Plaques may be counted to determine the viral load.

An alternative method for determining viral load is quantitative real-time PCR (qRT-PCR). Viral load can be determined by qRT-PCR using oligonucleotide primers specific for the RSV-F gene as described (I. Borg et al, Eur Respir J 2003; 21:944-51); the oligonucleotide primers may comprise some modifications. Methods for performing qRT-PCR are known in the art.

### VII. Immunogenic Compositions/Vaccines and Modes of Administration

Meta-stable protein-self-assembling molecule fusions (e.g., RSV F-ferritin fusion proteins) and nanoparticles of the present invention are capable of eliciting an immune response against the meta-stable protein, or infectious agent expressing the meta-stable protein. In one embodiment, the meta-stable protein is the RSV F protein in the pre-fusion conformation, and the self-assembling molecule is ferritin. Thus, these fusion proteins can be used as vaccines to protect individuals against, e.g., RSV infection. For exemplary purposes only, the passages below refer to an RSV F-ferritin fusion protein, but it will be understood by those of ordinary skill that the immunogenic compositions, vaccines, and modes of administration apply to any meta-stable protein-self-assembling molecule fusion of the present invention.

According to the present invention, a RSV F-ferritin fusion protein or nanoparticle can be used in an immunogenic composition to generate a vaccine. Thus, one embodiment of the present invention is a vaccine which includes a nanoparticle comprising an RSV F-ferritin fusion protein. Vaccines of the present invention can also contain other components such as adjuvants, buffers and the like. Adjuvants are described in further detail infra.

In one embodiment, the invention relates to a method of producing a vaccine against RSV, the method comprising a) expressing a complex comprising a monomeric self-assembly molecule and an RSV F protein under conditions such that F protein trimers in a pre-fusion conformation are displayed on the surface of a shell formed by polymerization of the self-assembly molecule, and b) recovering the shell displaying the F protein.

In one aspect, the invention relates to a method of vaccinating a subject against RSV, in particular, by targeting the virus before fusion to the host cell occurs. Such methods employ the vaccines of the present invention. Accordingly, in one embodiment, the method comprises administering a nanoparticle of the invention to a subject such that an immune response against RSV virus is produced in the subject, wherein the nanoparticle comprises RSV F protein, or fragment thereof, and a ferritin, wherein the ferritin forms a polymeric assembly that captures the RSV F protein or fragment thereof in a meta-stable pre-fusion conformation, wherein RSV F protein homotrimers in a pre-fusion conformation are displayed on the surface of a shell formed by polymeric assembly of the ferritin.

In one embodiment, the nanoparticle is comprised of a self-assembling molecule which assembles into a shell with polyhedral symmetry, such as an octahedral symmetry (as in, e.g., ferritin and HSP). In some embodiments, the shell comprises twenty four monomers of the self-assembling molecule.

In one embodiment, the RSV F protein is capable of eliciting neutralizing antibodies to RSV prior to fusion to a host cell by targeting the RSV F protein in the pre-fusion conformation.

Vaccines of the present invention can be used to vaccinate individuals using a prime/boost protocol. Such a protocol is described in U.S. Patent Publication No. 2011/0177122, which is incorporated herein by reference in its entirety. In such a protocol, a first vaccine composition may be administered to the individual (prime) and then after a period of time, a second vaccine composition may be administered to the individual (boost). Administration of the boosting composition is generally weeks or months after administration of the priming composition, preferably about 2-3 weeks or 4 weeks, or 8 weeks, or 16 weeks, or 20 weeks, or 24 weeks, or 28 weeks, or 32 weeks. In one embodiment, the boosting composition is formulated for administration about 1 week, or 2 weeks, or 3 weeks, or 4 weeks, or 5 weeks, or 6 weeks, or 7 weeks, or 8 weeks, or 9 weeks, or 16 weeks, or 20 weeks, or 24 weeks, or 28 weeks, or 32 weeks after administration of the priming composition

The first and second vaccine compositions can be, but need not be, the same composition. Thus, in one embodiment of the present invention, the step of administering the vaccine comprises administering a first vaccine composition, and then at a later time, administering a second vaccine composition. In one embodiment, the first vaccine composition comprises a nanoparticle comprising an RSV F-ferritin fusion protein of the present invention. In one embodiment, the RSV-F of the first vaccine composition comprises a nanoparticle comprising an RSV F-ferritin fusion protein which has an amino acid sequence at least about 80% identical, such as at least 85% identical, at least 90% identical, at least 95% identical, at least 97% identical or at least 99% identical to an amino acid sequence selected from the group consisting of SEQ ID NOs: 18-21, 24-27, and 30-33, wherein the nanoparticle elicits an immune response against RSV. In one embodiment, second vaccine composition comprises a nanoparticle comprising an identical RSV F-ferritin fusion protein as that of the first vaccine.

In one embodiment, the individual is at risk for infection with RSV. In one embodiment, the individual has been exposed to RSV. For example, the individual may be an elderly individual, a child, an infant or an immunocompromised individual. As used herein, the terms exposed, exposure, and the like, indicate the subject has come in contact with a person or animal that is known to be infected with RSV. Vaccines of the present invention may be administered using techniques well known to those in the art. Techniques for formulation and administration may be found, for example, in “Remington's Pharmaceutical Sciences”, 18^{th} ed., 1990, Mack Publishing Co., Easton, Pa. Vaccines may be administered by means including, but not limited to, traditional syringes, needleless injection devices, or microprojectile bombardment gene guns. Suitable routes of administration include, but are not limited to, parenteral delivery, such as intramuscular, intradermal, subcutaneous, intramedullary injections, as well as, intrathecal, direct intraventricular, intravenous, intraperitoneal, intranasal, or intraocular injections, just to name a few. For injection, the compounds of one embodiment of the invention may be formulated in aqueous solutions, preferably in physiologically compatible buffers such as Hanks' solution, Ringer's solution, or physiological saline buffer.

In one embodiment, vaccines, or nanoparticles, of the present invention can be used to protect s subject against infection by RSV. That is, a vaccine made using RSV-F protein from one strain of RSV is capable of protecting an individual against infection by different strains, e.g., mutant strains, of RSV.

In one embodiment, vaccines, or nanoparticles, of the present invention can be used to protect an individual against infection by an antigenically divergent RSV. Antigenically divergent refers to the tendency of a strain of RSV to mutate over time, thereby changing the amino acids that are displayed to the immune system.

### VIII. Adjuvants

The immunogenic compositions and vaccine compositions of the invention can be administered with one or more adjuvants. The use of adjuvants is routine in vaccine biology and one of ordinary skill would readily understand which adjuvant or combination of adjuvants are appropriate for a given vaccine.

Suitable adjuvants include, but are not limited to, those described in US2011/0305727 (herein incorporated by reference in its entirety).

In some embodiments, the adjuvant is a mineral-containing composition. Mineral-containing compositions suitable for use as adjuvants in the invention include mineral salts, such as calcium salts and aluminum salts (or mixtures thereof). The invention includes mineral salts such as hydroxides (e.g. oxyhydroxides), phosphates (e.g. hydroxyphosphates, orthophosphates), sulphates, etc., or mixtures of different mineral compounds, with the compounds taking any suitable form (e.g. gel, crystalline, amorphous, etc.), and with adsorption being preferred. Calcium salts include calcium phosphate (e.g., the “CAP” particles disclosed in US6355.271). Aluminum salts include hydroxides, phosphates, sulfates, and the like. The mineral containing compositions may also be formulated as a particle of metal salt (WO00/23105). Aluminum salt adjuvants are described in detail in US2011/0305727.

In some embodiments, the adjuvant is an oil emulsion compositions (described in detail in US2011/0305727). Oil emulsion compositions suitable for use as adjuvants in the invention include squalene-water emulsions, such as MF59 (5% Squalene, 0.5% Tween 80 and 0.5% Span, formulated into submicron particles using a microfluidizer).

In some embodiments, the adjuvant is a cytokine-inducing agent (described in detail in US2011/0305727). Cytokine-inducing agents suitable for use in the invention include toll-like receptor 7 (TLR7) agonists (e.g. benzonaphthyridine compounds disclosed in WO 2009/111337.

In some embodiments, the adjuvant is a saponin (chapter 22 of Vaccine Design: the Subunit and Adjuvant Approach (eds. Powell & Newman) Plenum Press 1995 (ISBN 0-306-44867-X)), which are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs. QS21 is marketed as STIMULON™. Saponin compositions have been purified using HPLC and RP-HPLC. Specific purified fractions using these techniques have been identified, including QS7, QS17, QS18, QS21, QH-A, QH-B and QH-C. Preferably, the saponin is QS21. A method of production of QS21 is disclosed in U.S. Pat. No. 5,057,540. Saponin formulations may also comprise a sterol, such as cholesterol (WO96/33739). Combinations of saponins and cholesterols can be used to form unique particles called immunostimulating complexes (ISCOMs). ISCOMs typically also include a phospholipid such as phosphatidylethanolamine or phosphatidylcholine. Any known saponin can be used in ISCOMs. Preferably, the ISCOM includes one or more of QuilA, QHA & QHC. ISCOMs are further described in WO96/33739, EP-A-0109942, and WO96/11711. Optionally, the ISCOMS may be devoid of additional detergent (WO00/07621). A review of the development of saponin based adjuvants can be found in Barr et al. (Advanced Drug Delivery Reviews 1998; 32:247-71) and Sjolanderet et al. (Advanced Drug Delivery Reviews 1998; 32:321-38).

In some embodiments, the adjuvant is a lipid-based adjuvant (described in detail in US2011/0305727), including oil-in-water emulsions, modified natural lipid As derived from enterobacterial lipopolysaccharides, phospholipid compounds (such as the synthetic phospholipid dimer, E6020) and the like.

In some embodiments, the adjuvant is a bacterial ADP-ribosylating toxin (e.g., the E. coli heat labile enterotoxin “LT”, cholera toxin “CT”, or pertussis toxin “PT”) and detoxified derivatives thereof, such as the mutant toxins known as LT-K63 and LT-R72 (Pizza et al., Int J Med Microbiol 2000; 290:455-61). The use of detoxified ADP-ribosylating toxins as mucosal adjuvants is described in WO95/17211 and as parenteral adjuvants in WO98/43275.

In some embodiments, the adjuvant is a bioadhesives or mucoadhesives, such as esterified hyaluronic acid microspheres (Singh et al., J Cont Release 2001; 70:267-76) or chitosan and its derivatives (WO99/27960).

In some embodiments, the adjuvant is a microparticle (i.e., a particle of ^{˜}100 nm to ^{˜}150 μm in diameter, more preferably ^{˜}200 nm to ^{˜}30 μm in diameter, or ^{˜}500 nm to ^{˜}10 μm in diameter) formed from materials that are biodegradable and non-toxic (e.g., a poly(α-hydroxy acid), a polyhydroxybutyric acid, a polyorthoester, a polyanhydride, a polycaprolactone, and the like), with poly(lactide-co-glycolide) being preferred, optionally treated to have a negatively-charged surface (e.g., with SDS) or a positively-charged surface (e.g., with a cationic detergent, such as CTAB).

In some embodiments, the adjuvant is a liposome (Chapters 13 & 14 of Vaccine Design: the Subunit and Adjuvant Approach (eds. Powell & Newman) Plenum Press 1995 (ISBN 0-306-44867-X)). Examples of liposome formulations suitable for use as adjuvants are described in U.S. Pat. No. 6,090,406, U.S. Pat. No. 5,916,588, and EP-A-0626169.

In some embodiments, the adjuvant is a polyoxyethylene ethers or polyoxyethylene ester (WO99/52549). Such formulations further include polyoxyethylene sorbitan ester surfactants in combination with an octoxynol (WO01/21207) as well as polyoxyethylene alkyl ethers or ester surfactants in combination with at least one additional non-ionic surfactant such as an octoxynol (WO01/21152). Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

In some embodiments, the adjuvant is a muramyl peptide, such as N-acetylmuramyl-L-threonyl-D-isoglutamine (“thr-MDP”), N-acetyl-normuramyl-L-alanyl-D-isoglutamine (nor-MDP), N-acetylglucsaminyl-N-acetylmuramyl-L-Al-D-isoglu-L-Ala-dipalmitoxy propylamide (“DTP-DPP”, or “Theramide™), N-acetylmuramyl-L-alanyl-D-isoglutaminyl-L-alanine-2-(1′-2′ dipalmitoyl-sn-glycero-3-hydroxyphosphoryloxy)-ethylamine (“MTP-PE”).

In some embodiments, the adjuvant is an outer membrane protein proteosome preparation prepared from a first Gram-negative bacterium in combination with a liposaccharide preparation derived from a second Gram-negative bacterium, wherein the outer membrane protein proteosome and liposaccharide preparations form a stable non-covalent adjuvant complex. Such complexes include “IVX-908”, a complex comprised of Neisseria meningitidis outer membrane and lipopolysaccharides.

In some embodiments, the adjuvant is a polyoxidonium polymer (Dyakonova et al., Int Immunopharmacol 2004; 4:1615-23; FR-2859633) or other N-oxidized polyethylene-piperazine derivative.

In some embodiments, the adjuvant is methyl inosine 5′-monophosphate (“MIMP”) (Signorelli & Hadden. Int Immunopharmacol 2003; 3:1177-86).

In some embodiments, the adjuvant is a polyhydroxlated pyrrolizidine compound described in WO2004/064715

In some embodiments, the adjuvant is a CD1d ligand, such as an α-glycosylceramide (De Libero et al. (Nature Reviews Immunology 2005; 5:485-96; U.S. Pat. No. 5,936,076; Oki et al. (J Clin Invest 2004; 113:1631-40); US2005/0192248; Yang et al. (Angew Chem Int Ed. 2004; 43:3818-22; WO2005/102049; Goffet et al. (Am Chem Soc 2004; 126:13602-3; WO03/105769) (e.g., α-galactosylceramide), phytosphingosine-containing α-glycosylceramides, OCH, KRN7000 [(2S,3S,4R)-1-O-(α-D-galactopyranosyl)-2-(N-hexacosanoylamino)-1,3,4-octadecanetriol], CRONY-101, 3″-O-sulfo-galactosylceramide, etc.

In some embodiments, the adjuvant is a gamma inulin (Cooper et al. Pharm Biotechnol 1995; 6:559-80) or derivative thereof, such as algammulin.

In some embodiments, the adjuvant is a virosome or virus-like particle (VLP). These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). Accordingly, in some embodiments, the protein cage itself may also function as the adjuvant.

These and other adjuvant-active substances are discussed in more detail in. e.g., Vaccine Design: The Subunit and Adjuvant Approach (eds. Powell & Newman) Plenum Press 1995 (ISBN 0-306-44867-X) and Vaccine Adjuvants: Preparation Methods and Research Protocols (Volume 42 of Methods in Molecular Medicine series). ISBN: 1-59259-083-7. Ed. O'Hagan.

The immunogenic compositions of the present invention may include two, three, four or more adjuvants. For example, compositions of the invention may advantageously include both an oil-in-water emulsion and a cytokine-inducing agent, or both a mineral-containing composition and a cytokine-inducing agent, or two oil-in-water emulsion adjuvants, or two benzonaphthyridine compounds, etc.

The use of an aluminum hydroxide and/or aluminum phosphate adjuvant is useful, particularly in children, and antigens are generally adsorbed to these salts. Squalene-in-water emulsions are also preferred, particularly in the elderly. Useful adjuvant combinations include combinations of Th1 and Th2 adjuvants such as CpG and alum, or resiquimod and alum. A combination of aluminum phosphate and 3dMPL may be used. Other combinations that may be used include: alum and a benzonapthridine compound or a SMIP, a squalene-in-water emulsion (such as MF59) and a benzonapthridine compound or a SMIP, and E6020 and a squalene-in-water emulsion, such as MF59) or alum.

Additional suitable adjuvants include: genetic adjuvants such as IL-2 gene or fragments thereof, the granulocyte macrophage colony-stimulating factor (GM-CSF) gene or fragments thereof, the IL-18 gene or fragments thereof, the chemokine (C-C motif) ligand 21 (CCL21) gene or fragments thereof, the IL-6 gene or fragments thereof; and other immune stimulatory genes; protein adjuvants such IL-2 or fragments thereof, the granulocyte macrophage colony-stimulating factor (GM-CSF) or fragments thereof, IL-18 or fragments thereof, the chemokine (C-C motif) ligand 21 (CCL21) or fragments thereof, IL-6 or fragments thereof, lipid adjuvants such as cationic liposomes, N3 (cationic lipid), monophosphoryl lipid A (MPL1); other adjuvants including Fms-like tyrosine kinase-3 ligand (Flt-3L), bupivacaine, marcaine, and levamisole.

The compositions of the invention may elicit both a cell mediated immune response as well as a humoral immune response. A TH1 immune response may be elicited using a TH1 adjuvant. A TH1 adjuvant will generally elicit increased levels of IgG2a production relative to immunization of the antigen without adjuvant. TH1 adjuvants suitable for use in the invention may include for example saponin formulations, virosomes and virus like particles, non-toxic derivatives of enterobacterial lipopolysaccharide (LPS), immunostimulatory oligonucleotides. Immunostimulatory oligonucleotides, such as oligonucleotides containing a CpG motif, are preferred TH1 adjuvants for use in the invention. A TH2 immune response may be elicited using a TH2 adjuvant. A TH2 adjuvant will generally elicit increased levels of IgG1 production relative to immunization of the antigen without adjuvant. TH2 adjuvants suitable for use in the invention include, for example, mineral containing compositions, oil-emulsions, and ADP-ribosylating toxins and detoxified derivatives thereof. Mineral containing compositions, such as aluminium salts are preferred TH2 adjuvants for use in the invention.

A composition may include a combination of a TH1 adjuvant and a TH2 adjuvant. Preferably, such a composition elicits an enhanced TH1 and an enhanced TH2 response, i.e., an increase in the production of both IgG1 and IgG2a production relative to immunization without an adjuvant. Still more preferably, the composition comprising a combination of a TH1 and a TH2 adjuvant elicits an increased TH1 and/or an increased TH2 immune response relative to immunization with a single adjuvant (i.e., relative to immunization with a TH1 adjuvant alone or immunization with a TH2 adjuvant alone).

### IX. Methods of Prophylaxis and Treatment

Methods of preparing and administering immunogenic compositions to a subject in need thereof are well known in the art or readily determined by those skilled in the art. The dosage and frequency of administration may depend on whether the treatment is prophylactic or therapeutic.

The immunogenic composition and nanoparticles of the invention are suitable for administration to mammals (e.g., primates, (e.g., humans, chimpanzees, monkeys, baboons), rats (e.g., cotton rats), mice, cows (e.g., calves), guinea pigs, ferrets and hamsters). In one embodiment, the invention provides a method of inducing an immune response in a mammal, comprising the step of administering a composition (e.g., an immunogenic composition) of the invention to the mammal. The compositions (e.g., an immunogenic composition) can be used to produce a vaccine formulation for immunizing a mammal. The mammal is typically a human, and the immunogenic composition typically comprises an RSV F-ferritin fusion protein or an RSV F-HSP fusion protein. However, the mammal can be any other mammal that is susceptible to infection with RSV, such as a cow that can be infected with bovine RSV.

The invention also provides a composition of the invention for use as a medicament, e.g., for use in immunizing a patient against RSV infection.

The invention also provides the use of a polypeptide as described above in the manufacture of a medicament for raising an immune response in a patient.

The immune response raised by these methods and uses will generally include an antibody response, preferably a protective antibody response. Methods for assessing antibody responses after RSV vaccination are well known in the art.

Compositions of the invention can be administered in a number of suitable ways, such as intramuscular injection (e.g., into the arm or leg), subcutaneous injection, intranasal administration, oral administration, intradermal administration, transcutaneous administration, transdermal administration, and the like. The appropriate route of administration will be dependent upon the age, health and other characteristics of the mammal A clinician will be able to determine an appropriate route of administration based on these and other factors.

Immunogenic compositions, and vaccine formulations, may be used to treat both children and adults, including pregnant women. Thus a subject may be less than 1 year old, 1-5 years old, 5-15 years old, 15-55 years old, or at least 55 years old. Preferred subjects for receiving the vaccines are the elderly (e.g., >50 years old, >60 years old, >65 years, and preferably >75 years), the young (e.g., <6 years old, such as 4-6 years old, <5 years old), and pregnant women. The vaccines are not limited to these groups, however, and may be used more generally in a population.

Treatment can be by a single dose schedule or a multiple dose schedule. Multiple doses may be used in a primary immunization schedule and/or in a booster immunization schedule. In a multiple dose schedule the various doses may be given by the same or different routes, e.g., a parenteral prime and mucosal boost, a mucosal prime and parenteral boost, etc. Administration of more than one dose (typically two doses) is particularly useful in immunologically naive patients. Multiple doses will typically be administered at least 1 week apart (e.g., about 2 weeks, about 3 weeks, about 4 weeks, about 6 weeks, about 8 weeks, about 10 weeks, about 12 weeks, about 16 weeks, and the like.)

Vaccine formulations produced using a composition of the invention may be administered to patients at substantially the same time as (e.g., during the same medical consultation or visit to a healthcare professional or vaccination center) other vaccines.

### X. Kits

The immunogenic composition or nanoparticle of the invention can be provided in a kit. In one embodiment, the kit includes (a) a container that contains a composition that includes one or more unit doses of the immunogenic composition or nanoparticle, and optionally (b) informational material. The unit doses of the immunogenic composition or nanoparticle are sufficient to cause an immunogenic response (e.g., antibody production) in a subject. The informational material can be descriptive, instructional, marketing or other material that relates to the methods described herein and/or the use of the agents for therapeutic benefit. The kit can also include reagents and instructions useful in the testing (assaying) for an immunogenic response. Such methods of assaying for an immunogenic response include, but are not limited to, any of the testing methods described herein. In one embodiment, the kit includes one or more additional agents for treating RSV. For example, the kit includes a first container that contains a composition that includes the immunogenic composition, and a second container that includes the one or more additional agents.

The informational material of the kits is not limited in its form. In one embodiment, the informational material can include information about production of the immunogenic composition, molecular weight of the composition, concentration, date of expiration, batch or production site information, and so forth.

In one embodiment, the informational material relates to methods of administering the immunogenic composition, e.g., in a suitable dose, dosage form, or mode of administration (e.g., a dose, dosage form, or mode of administration described herein), to treat a subject who is infected with RSV, or who is at risk of being infected with RSV. The information can be provided in a variety of formats, including printed text, computer readable material, video recording, or audio recording, or information that provides a link or address to substantive material.

In addition to the agent (e.g., RSV F-ferritin fusion protein), the composition in the kit can include other ingredients, such as a solvent or buffer, a stabilizer, or a preservative. The agent can be provided in any form, e.g., liquid, dried or lyophilized form, preferably substantially pure and/or sterile. When the agents are provided in a liquid solution, the liquid solution preferably is an aqueous solution. When the agents are provided as a dried form, reconstitution generally is by the addition of a suitable solvent. The solvent, e.g., sterile water or buffer, can optionally be provided in the kit.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes both the RSV F-ferritin fusion protein and the second agent, e.g., in a desired ratio. For example, the kit includes a plurality of syringes, ampules, foil packets, blister packs, or medical devices, e.g., each containing a single combination unit dose. The containers of the kits can be air tight, waterproof (e.g., impermeable to changes in moisture or evaporation), and/or light-tight.

The kit optionally includes a device suitable for administration of the composition, e.g., a syringe or other suitable delivery device. The device can be provided pre-loaded with one or both of the agents or can be empty, but suitable for loading.

All publications, patents, and patent applications cited herein, whether supra or infra, are hereby incorporated by reference in their entirety.

### EXAMPLES

### Example 1

### Design of Nanoparticle and Creation of Model

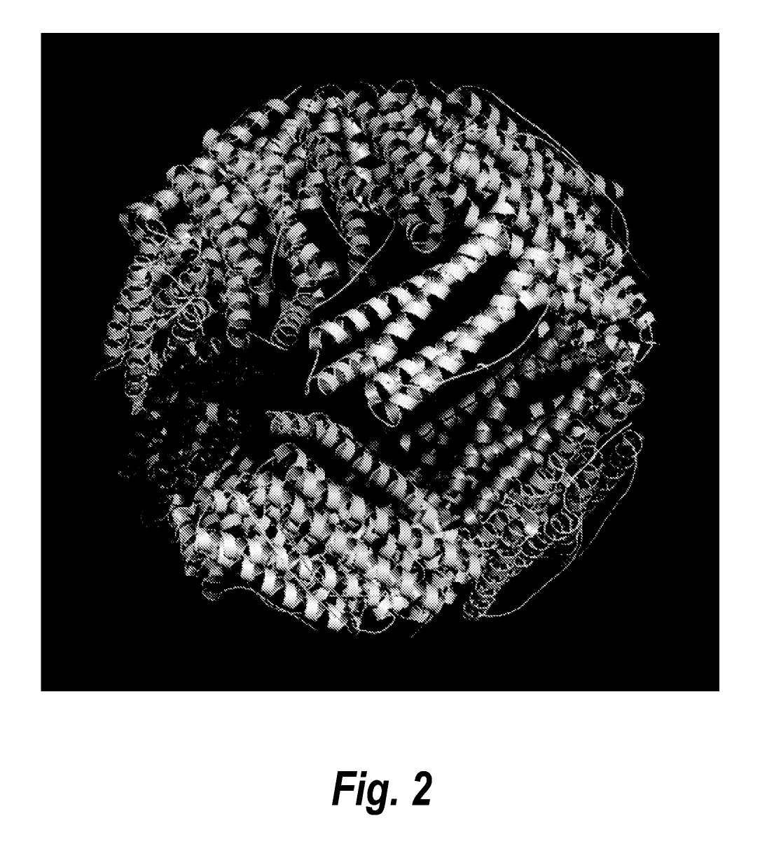

In nature, ferritin and heat shock proteins are two examples of proteins that are well known to self-assemble under appropriate conditions into a shell with polyhedral symmetry. The ferritin shells are composed of twenty-four ferritin monomers that occupy each of the twenty-four symmetry domains of an octahedral symmetry (FIG. 2).

An exemplary meta-stable viral protein, F glycoprotein of RSV, in its pre-fusion state adopts a trimeric quaternary structure. Another exemplary meta-stable viral protein, E glycoprotein of Dengue, adopts a trimeric quaternary structure in a pre-fusion intermediate state. The conformation of the meta-stable viral proteins in these states is favorable for implementing effective nanoparticle vaccines. Molecules that naturally form dimers and trimers can be arranged on the two- or three-fold axes respectively of the polyhedral shell. If they are attached appropriately, the quaternary structure interactions within these dimers and trimers can aid in the assembly of the polyhedral shell.

A key consideration in the design of nanoparticles is identifying the exact amino acid residue on both the meta-stable viral protein as well as the self-assembling protein and the precise linker which will attach the two molecules such that the resultant molecule will self assemble into a nanoparticle with polyhedral symmetry and the meta-stable viral protein is locked in the pre-fusion conformation. Computational modeling of the two protein systems was used to accomplish this. In addition, the specific experimental conditions required to create the nanoparticle with these properties must be determined. Presented below are the details of the computational modeling to generate the composition of a RSV F glycoprotein and ferritin nanoparticle. It will be understood by those of ordinary skill that the F glycoprotein-ferritin combination described below is merely exemplary and that the method can be applied to any meta-stable protein-self-assembling molecule combination.

### Model Generation

Computational modeling of the molecular structure of the RSV pre-fusion F glycoprotein and ferritin was performed to identify the exact residues of the two proteins which will be linked and precise composition and structure of the linker that will connect them in such a manner so that the self-assembly of ferritin will occur and RSV F glycoprotein will remain and be locked in its pre-fusion conformation.

The pre-fusion F glycoprotein is a trimer, so it was hypothesized that twenty-four monomers composed of ferritin attached to the F protein would assemble with three F proteins oriented around each of the three-fold symmetry axes.

a. The Shell

An important consideration when creating the model of the nanoparticle was the orientation of the three-fold symmetry axes of the trimerized pre-fusion F protein and three of the ferritin molecules in the shell (FIG. 4).

b. The Linker

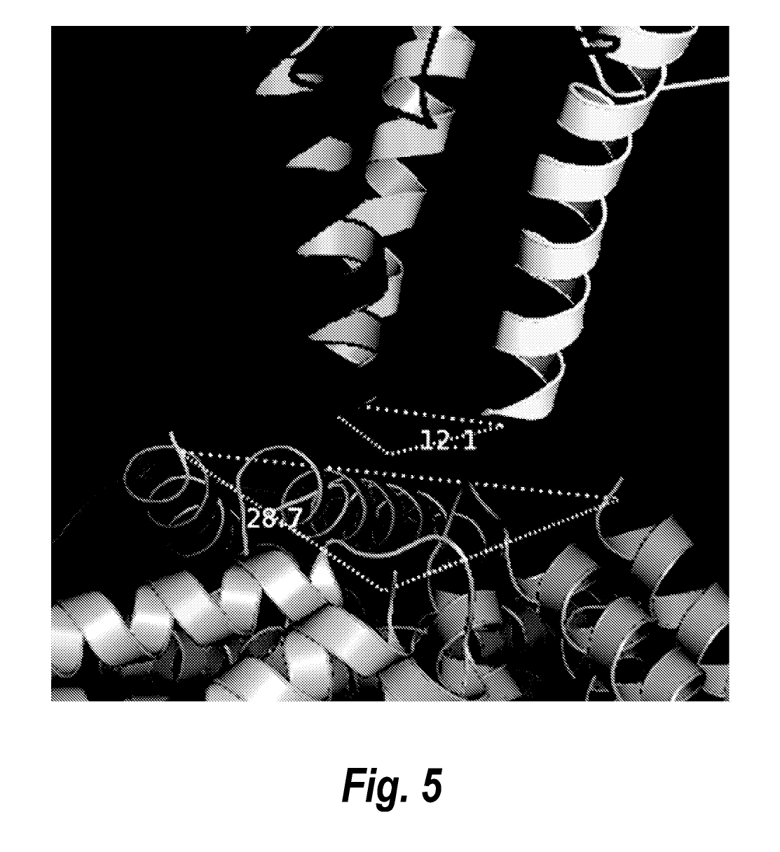

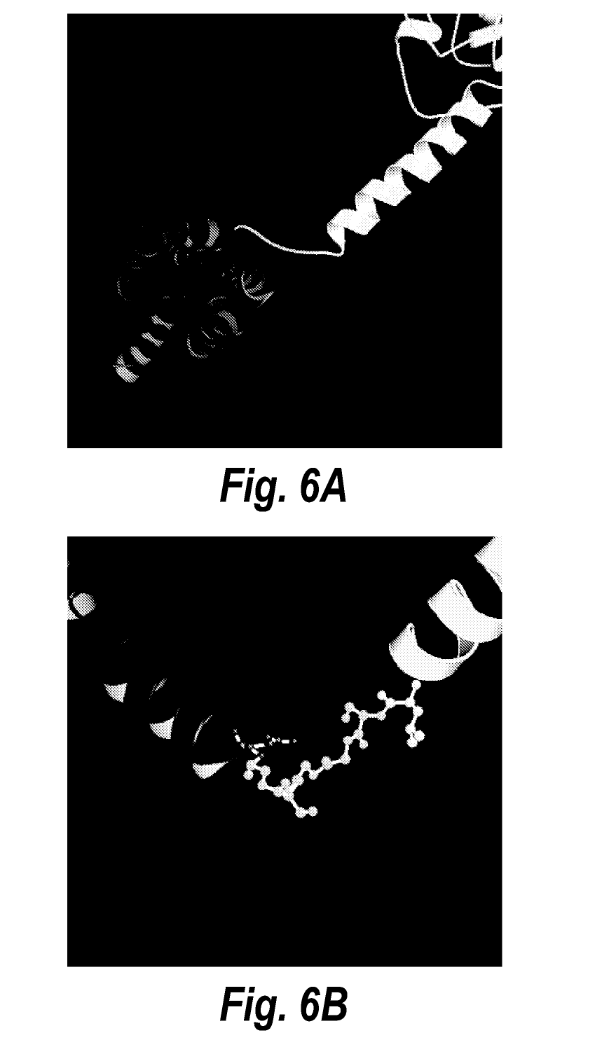

In order to orient the F protein trimers and ferritin trimers about the same three-fold axis, the Leu-513 residue of the F protein may be linked to the Asp-5 residue of the ferritin. Connecting each of these sets of residues formed two equilateral triangles with side lengths of 12.1 angstroms for the F protein trimer and 28.7 angstroms for the ferritin trimer (FIG. 5). If these triangles were in the same plane, the distance between the Leu-513 of an F protein and the ASP-5 of a ferritin would be 9.6 angstroms. However, it is necessary to raise the F protein trimer above the plane of the ferritin trimer, in order ensure that there are no steric hindrances and that the self-assembly of the ferritin can occur. This suggests a linker length of about 4-7 amino acid residues. The composition of the linker could be alternating serine and glycine residues, allowing for flexibility in its conformation (FIGS. 6A and 6B). Exemplary F protein-linker-ferritin fusions with a 7-amino acid linker (SGGSGSG; SEQ ID NO: 48), 6-amino acid linker (SGSGSG; SEQ ID NO: 47), 5-amino acid linker (SGGSG; SEQ ID NO: 45), and a 4-amino acid linker (SGSG; SEQ ID NO: 43) are set forth in SEQ ID NOs: 18-21, 24-27, and 30-33, respectively.

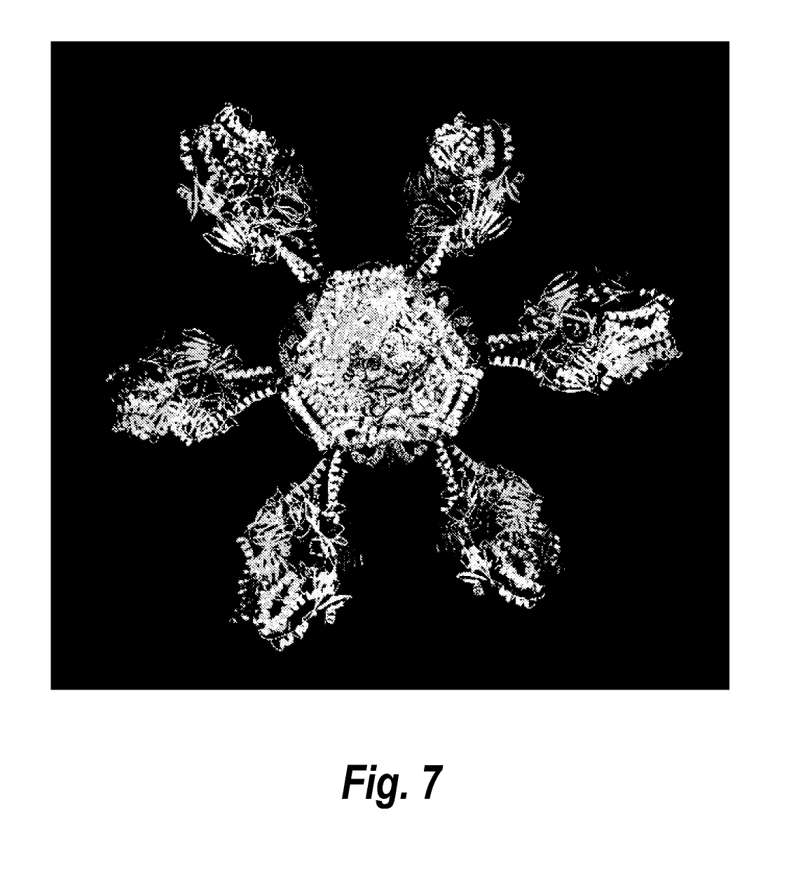

The resulting structure was verified and refined to ensure that there were no bad contacts or phi/psi violations. Finally, eight of these aligned trimers were aligned as per the self-assembly template of ferritin, forming an octahedral shell of ferritin/F protein monomers, wherein the F protein trimers are presented and locked in the pre-fusion conformation (FIG. 7).

### Example 2

### Expression and Purification of RSVF-Linker-Helicobacter pylori Ferritin (HypF) Fusion Protein

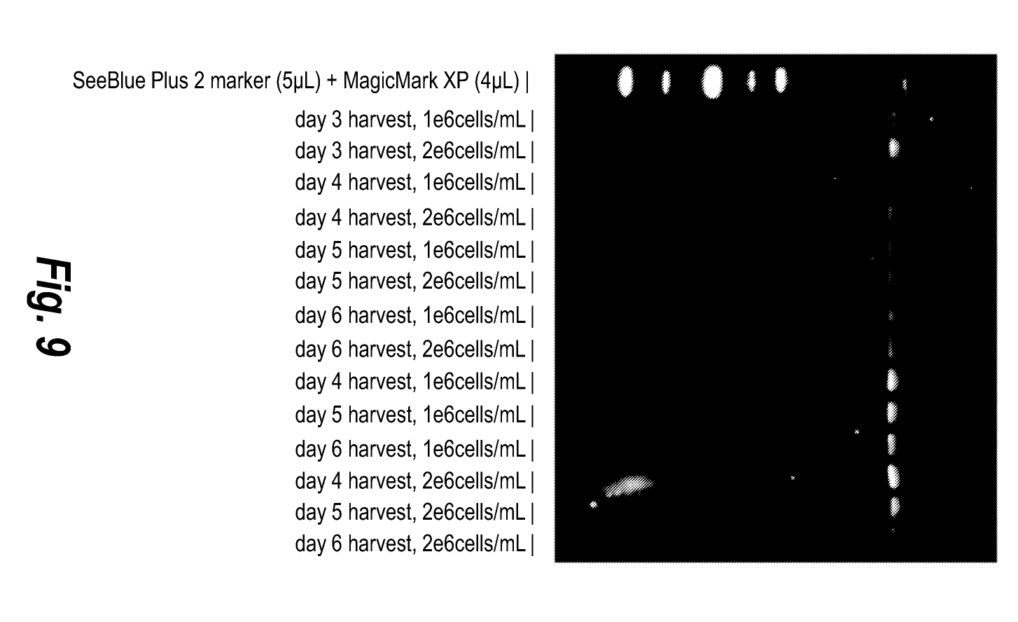

An RSVF-linker-HypF fusion construct with a 4 amino acid linker (SGSG; SEQ ID NO: 43) and a human CD5 leader sequence (MPMGSLQPLATLYLLGMLVASCLG; SEQ ID NO: 51) was codon optimized for mammalian expression and transiently transfected into 293F cells plated at two different densities (1×10⁶ and 2×10⁶ cells/mL) using PEI. Protein was harvested from the culture supernatant on days 3, 4, 5, and 6 post-transfection and analyzed by Western blot. Proteins were detected using the monoclonal D25 antibody (SOURCE; 1:1000), which specifically recognizes the F protein in the pre-fusion state, and a secondary rabbit anti-human Fcγ antibody (SOURCE; 1:10,000). As shown in FIG. 9, the RSVF-HypF fusion protein was detectable, and the F protein was expressed in the pre-fusion state. The molecular weight of the fusion protein suggests that it was expressed as a trimer.

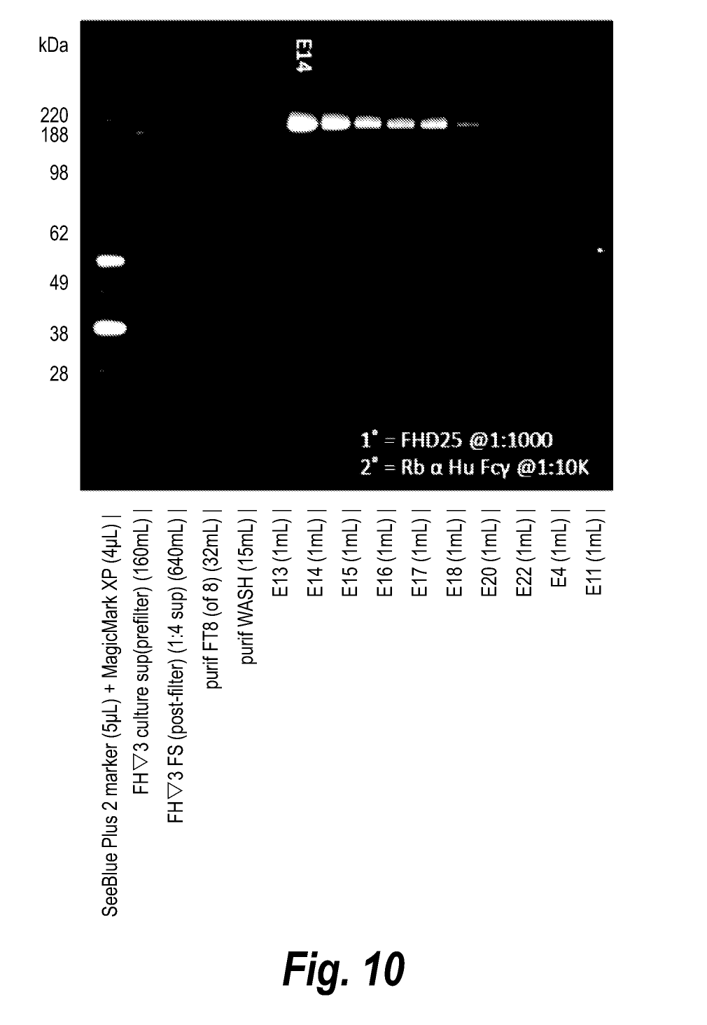

The RSVF-HypF fusion protein was also purified by FPLC using an anion exchange column (HiTrap Q HP column; GE). Culture supernatants of 293F cells transfected as described above were collected, diluted 1:4 in Freestyle 293 media (SOURCE), and loaded onto an anion exchange column. The column was washed with 1×PBS and eluted using a NaCl gradient (1×PBS+1M NaCl). Aliquots of the eluted fractions were subjected to Western blot using the monoclonal D25 antibody, as described above. As shown in FIG. 10, no protein was detected in either the flow through or wash fractions. In all eluted fractions containing the RSVF-HypF protein, the fusion was detected with the D25 antibody, suggesting that the F protein was expressed in the pre-fusion state. Again, the molecular weight of the fusion protein suggests that it was expressed as a trimer.

These results collectively suggest that an RSVF-ferritin fusion protein designed according to the method in Example 1, is expressed with the F protein in the pre-fusion state.

### Example 3

### RSV F Protein—HSP Nanoparticle

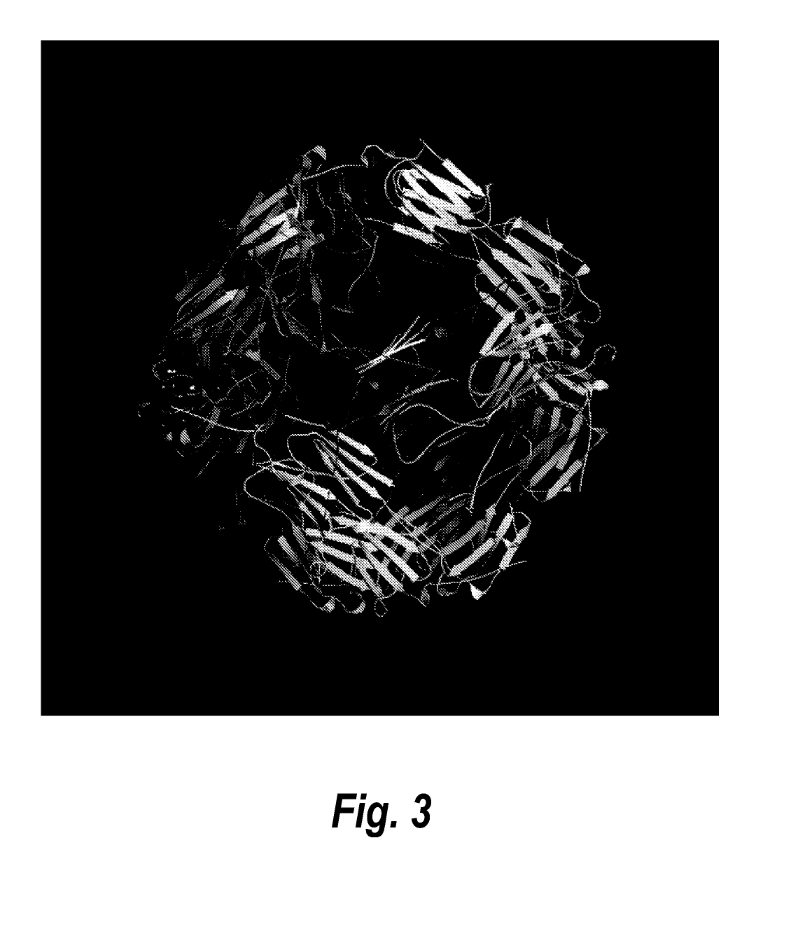

A similar method was also used to generate the composition of a RSV F glycoprotein and heat shock protein nanoparticle. Heat shock proteins are another example of proteins that are well known to self-assemble under appropriate conditions into a shell with polyhedral symmetry (FIG. 3). Accordingly, a similar method to that described above for ferritin-F protein nanoparticles was used to design RSV F protein-small HSP20 nanoparticles that would stabilize the F protein in a pre-fusion conformation. Computational modeling as described in Example 1 showed that in order to orient the F protein trimers and sHSP20 trimers about the same three-fold axis, the Leu-513 residue of the F protein can be linked to the Thr-24 residue of sHSP20. The optimal linker length was found to be 8-10 amino acids. An exemplary F protein-linker-sHSP20 protein fusion with a 10-amino acid linker (SGSGSGSGSG; SEQ ID NO: 50) is set forth in SEQ ID NO: 22.

### Example 4

### F Protein—Non Proteinaceous Self-Assembling Molecule

Similar considerations as those described in Example 1 can be applied to the use of non-proteinaceous self-assembling molecules, such as DNA. DNA can assemble into a wide range of one dimensional, two dimensional, or even three-dimensional structures, such as cubes, octahedra, tetrahedra, dodecahedra, or buckyballs, as described in, e.g., He et al., Nature 2008; 452:198-201. Accordingly, a similar computational modeling strategy described in Example 1 can be tailored to the design of an RSV F protein-DNA fusion nanoparticle, in which the trimeric RSV F protein is presented in a pre-fusion locked state on DNA nanostructures. Methods of linking proteins to nucleic acids are well known in the art. Such RSV F protein-DNA fusion nanoparticles can be tested for the ability to elicit antibodies against the pre-fusion F protein using the methods described herein.

### Example 5

### Immunization with RSV F Protein/Self-Assembling Molecule Fusion

The RSV F-self-assembling molecule fusions described herein (e.g., those described in Examples 1-4) and adjuvant are administered to a subject twice intramuscularly at two or three week intervals. Serum is collected from the subject two weeks after each immunization and one week prior to the first immunization.

Pre- and Post-immune sera from immunized subjects are assayed for binding to RSV F by ELISA. The sera is serially diluted and assayed for reactivity with RSV F protein as well as control proteins.

### Example 6

### Immunization and Challenge

Immunization and viral challenge of mice are carried out using the method described in Singh et al., Vaccine 2007; 25:6211-23. Briefly, 4-6 week old mice (e.g., BALB/c mice which are susceptible to infection by human RSV A2 strain) are immunized intranasally with the RSV F-self-assembling molecule fusions described herein or vehicle, and an adjuvant, three times (once every 2 weeks). Two weeks after the final immunization, mice are challenged with live RSV intranasally, and sacrificed 5 days after challenge. Lungs are collected, homogenized, and centrifuged to collect supernatant containing the virus. Supernatants are serially diluted 10-fold in PBS for titer determination. Serially diluted supernatant is added to HEp-2 cells grown on monolayers in chambered tissue culture slides, with adsorption carried out for 30 min, followed by addition of culture medium. RSV infection is carried out for 48 hours, followed by fixation of cells in 10% TCA and successive alcohol washes. Cells are then subjected to fluorescence immunocytochemistry using an anti-RSV F monoclonal antibody and FITC-conjugated goat anti-mouse secondary antibody. Slides are viewed under a fluorescent microscope and the number of cells with fluorescence is counted to obtain the number of PFU. PFU is then compared between mice immunized with the RSVF-ferritin fusion and vehicle. Mice immunized with the RSV F-self-assembling molecule fusions are expected to have a lower PFU than mice administered vehicle.

### EMBODIMENTS

1. A nanoparticle comprising Respiratory syncytial virus (RSV) F protein, or fragment thereof, and a self-assembling molecule, wherein the self-assembling molecule forms a polymeric assembly that captures the F protein or fragment thereof in a meta-stable pre-fusion conformation, thereby forming the nanoparticle.
2. The nanoparticle of embodiment 1, wherein the F protein comprises an F1 and F2 heterodimer.
3. The nanoparticle of embodiment 1, wherein the F protein fragment comprises an ectodomain.
4. The nanoparticle of embodiment 1, wherein the F protein fragment comprises a heptad-repeat A domain (HRA) and a heptad-repeat C domain (HRC).
5. The nanoparticle of embodiment 1, wherein the F protein fragment comprises an HRA domain, an HRC domain, and F1 domains I and II.
6. The nanoparticle of embodiment 1, wherein the F protein fragment comprises an HRA domain, an HRC domain, F1 domains I and II, and a heptad-repeat B domain (HRB).
7. The nanoparticle of embodiment 1, comprising one or more homotrimers of F1 and F2.
8. The nanoparticle of embodiment 1, wherein the F protein comprises an amino acid sequence set forth in SEQ ID NOs:1-12.
9. The nanoparticle of any of the preceding embodiments, wherein the F protein is covalently attached to the self-assembling molecule.
10. The nanoparticle of any of the preceding embodiments, wherein the F protein is genetically fused to the self-assembling molecule.
11. The nanoparticle of any one of the preceding embodiments, wherein the self-assembling molecule is selected from the group consisting of a protein, peptide, nucleic acid, a virus-like particle, a viral capsid, lipid, and carbohydrate.
12. The nanoparticle of embodiment 11, wherein the self-assembling molecule assembles into a shell with polyhedral symmetry.
13. The nanoparticle of embodiment 12, wherein the shell has an octahedral symmetry.
14. The nanoparticle of embodiment 13, wherein the shell comprises twenty four monomers of the self-assembling molecule.
15. The nanoparticle of embodiment 11, wherein the self-assembling molecule is a protein selected from the group consisting of ferritin, heat shock protein, and Dsp.
16. The nanoparticle of embodiment 15, wherein the self-assembling molecule is ferritin.
17. The nanoparticle of embodiment 16, wherein the ferritin protein comprises an amino acid sequence set forth in SEQ ID NO:13-17.
18. The nanoparticle of embodiment 15, wherein the self-assembling molecule is a heat shock protein, such as sHSP (small heat shock protein), HSP100, HSP90, HSP70, and HSP60.
19. The nanoparticle of embodiment 18, wherein the heat shock protein comprises an amino acid sequence set forth in SEQ ID NO:36-42.
20. The nanoparticle of any one of the preceding embodiments, wherein the F protein and self-assembling molecule are attached by means of a linker.
21. The nanoparticle of embodiment 20, wherein the linker is of sufficient length to prevent steric hindrance between the self-assembling molecule and F protein.
22. The nanoparticle of embodiment 20, wherein the linker is a gly-ser linker.
23. The nanoparticle of embodiment 22, wherein the linker is about 4 to 7 amino acids long.
24. The nanoparticle of any one of the preceding embodiments, wherein the amino acid sequence of the F protein further comprises an N-terminal leader that facilitates secretion from cells.
25. The nanoparticle of embodiments 24, wherein the N-terminal leader comprises an amino acid sequence selected from the group consisting of SEQ ID NOs: 51-53.
26. An immunogenic composition comprising the nanoparticle of any one of the preceding embodiments and a pharmaceutically acceptable carrier.
27. The immunogenic composition of embodiment 26, further comprising an adjuvant.
28. A vaccine composition comprising a nanoparticle according to any one of embodiments 1-25, wherein F protein homotrimers in a pre-fusion conformation are displayed on the surface of a shell formed by polymeric assembly of the self-assembly molecule.
29. A vaccine composition of embodiment 28, further comprising an adjuvant.
30. An RSV F protein-ferritin fusion protein comprising the amino acid sequence selected from the group consisting of SEQ ID NO: 18-21, 24-27, and 30-33.
31. An RSV F protein-heat shock protein fusion protein comprising the amino acid sequence selected from the group consisting of SEQ ID NO: 22, 23, 28, 29, 34, and 35.
32. A kit comprising the nanoparticle or fusion protein of any one of embodiments 1-25, 30, and 31, and instructions for use.
33. A method of producing an antibody which inhibits and/or prevents RSV infection comprising administering to a subject the nanoparticle, immunogenic composition, vaccine composition, or fusion protein of any one of embodiments 1-31.
34. The method of embodiment 33 further comprising isolating the antibody from the subject.
35. A method of producing a vaccine against RSV, the method comprising a) expressing a complex comprising a monomeric self-assembly molecule and an RSV F protein under conditions such that F protein trimers in a pre-fusion conformation are displayed on the surface of a shell formed by polymerization of the self-assembly molecule, and b) recovering the shell displaying the F protein.
36. A method of vaccinating a subject against RSV comprising administering to the subject a vaccine according to embodiment 35.
37. The method of embodiment 36, wherein the linker attachment point on the F protein is leucine at position 513 of SEQ ID NO: 1, and the linker attachment point on ferritin is aspartic acid at position 5 of SEQ ID NO: 13.
38. An isolated nucleic acid encoding the nanoparticle or fusion protein of any one of embodiments 1-25, 30, and 31.
39. A vector comprising the nucleic acid of embodiment 38.
40. An isolated cell comprising the nucleic acid of embodiment 39.
41. A nanoparticle comprising a viral fusion protein, or fragment thereof, and a self-assembling molecule, wherein the self-assembling molecule forms a polymeric assembly that captures the viral fusion protein in a meta-stable pre-fusion conformation thereby forming the nanoparticle.
42. The nanoparticle of embodiment 41, wherein the viral fusion protein is a class I, II, or III fusion protein.
43. The nanoparticle of embodiment 42, wherein the viral fusion protein adopts a dimeric or trimeric quaternary structure.
44. The nanoparticle of embodiment 43, wherein the viral fusion protein adopts a trimeric quaternary structure.
45. The nanoparticle of embodiment 41, wherein the viral fusion protein is a Paramyxoviridae, Flaviviridae, or Retroviridae viral fusion protein.
46. The nanoparticle of embodiment 45, wherein the Paramyxoviridae viral fusion protein is a Paramyxovirinae or Pneumonvirinae virus selected from the group consisting of Avularvirus, Respirovirus, and Pneumovirus.
47. The nanoparticle of embodiment 46, wherein the virus is selected from the group consisting of New Castle disease virus, Sendai virus, and Respiratory syncytial virus (RSV).
48. The nanoparticle of embodiment 47, wherein the virus is RSV and the fusion protein is F protein.
49. The nanoparticle of embodiment 45, wherein the Flaviviridae viral fusion protein is a Flavivirus.
50. The nanoparticle of embodiment 49, wherein the Flavivirus is West Nile virus, Dengue virus, or yellow fever virus.
51. The nanoparticle of embodiment 50, wherein the virus is Dengue virus and the fusion protein is E protein.
52. The nanoparticle of any of the embodiments 41-51, wherein the viral fusion protein is covalently attached to the self-assembling molecule.
53. The nanoparticle of any of embodiments 41-52, wherein the viral fusion protein is genetically fused to the self-assembling molecule.
54. The nanoparticle of any one of embodiments 41-53, wherein the self-assembling molecule is selected from the group consisting of a protein, peptide, nucleic acid, a virus-like particle, a viral capsid, lipid, and carbohydrate.
55. The nanoparticle of embodiment 54, wherein the self-assembling molecule assembles into a shell with polyhedral symmetry.
56. The nanoparticle of embodiment 55, wherein the shell has an octahedral symmetry.
57. The nanoparticle of embodiment 56, wherein the shell comprises twenty four monomers of the self-assembling molecule.
58. The nanoparticle of embodiment 57, wherein the self-assembling molecule is a protein selected from the group consisting of ferritin, heat shock protein, Dsp, lumazine synthase, and MrgA.
59. The nanoparticle of embodiment 58, wherein the self-assembling molecule is ferritin.
60. The nanoparticle of embodiment 49, wherein the ferritin protein comprises an amino acid sequence set forth in SEQ ID NO: 13-17.
61. The nanoparticle of embodiment 60, wherein the self-assembling molecule is a heat shock protein selected from the group consisting of sHSP, HSP100, HSP90, HSP70 and HSP60.
62. The nanoparticle of embodiment 61, wherein the heat shock protein comprises an amino acid sequence set forth in SEQ ID NO: 36-42.
63. The nanoparticle of any one of embodiments 41-62, wherein the viral fusion protein and self-assembling molecule are attached by means of a linker.
64. The nanoparticle of embodiment 63, wherein the linker is of sufficient length to prevent steric hindrance between the self-assembling molecule and viral fusion protein.
65. The nanoparticle of embodiment 64, wherein the linker is a (GlySer)n linker.
66. The nanoparticle of embodiment 65, wherein the linker is about 4 to 7 amino acids long.
67. An immunogenic composition comprising the nanoparticle of any one of embodiments 41-66, and a pharmaceutically acceptable carrier.
68. The immunogenic composition of embodiment 67, further comprising an adjuvant.
69. A vaccine composition comprising a nanoparticle according to any one of embodiments 41-66, wherein viral fusion protein homotrimers in a pre-fusion conformation are displayed on the surface of a shell formed by polymeric assembly of the self-assembly molecule.
70. A vaccine composition of embodiment 69, further comprising an adjuvant.

| SEQ | | |
|---|---|---|
| ID | Description | Sequence |
| 1 | RSV F protein from A2 strain | MELLILKANAITTILTAVTFCFASGQNITEEFYQSTCSAVSKGYLSALR |
| | (GenBank GI: 138251; Swiss | TGWYTSVITIELSNIKENKCNGTDAKVKLIKQELDKYKNAVTELQLLMQ |
| | Prot P03420) (full length) | STPPTNNRARRELPRFMNYTLNNAKKTNVTLSKKRKRRFLGFLLGVGSA |
| | | IASGVAVSKVLHLEGEVNKIKSALLSTNKAVVSLSNGVSVLTSKVLDLK |
| | | NYIDKQLLPIVNKQSCSISNIETVIEFQQKNNRLLEITREFSVNAGVTT |
| | | PVSTYMLTNSELLSLINDMPITNDQKKLMSNNVQIVRQQSYSIMSIIKE |
| | | EVLAYVVQLPLYGVIDTPCWKLHTSPLCTTNTKEGSNICLTRTDRGWYC |
| | | DNAGSVSFFPQAETCKVQSNRVFCDTMNSLTLPSEINLCNVDIFNPKYD |
| | | CKIMTSKTDVSSSVITSLGAIVSCYGKTKCTASNKNRGIIKTFSNGCDY |
| | | VSNKGMDTVSVGNTLYYVNKQEGKSLYVKGEPIINFYDPLVFPSDEFDA |
| | | SISQVNEKINQSLAFIRKSDELLHNVNAGKSTTNIMITTIIIVIIVILL |
| | | SLIAVGLLLYCKARSTPVTLSKDQLSGINNIAFSN |
| 2 | RSV F protein (C-terminal | MELLILKANAITTILTAVTFCFASGQNITEEFYQSTCSAVSKGYLSALR |
| | truncation aa 1-524, derived | TGWYTSVITIELSNIKENKCNGTDAKVKLIKQELDKYKNAVTELQLLMQ |
| | from A2 strain) | STPATNNRARRELPRFMNYTLNNAKKTNVTLSKKRKRRFLGFLLGVGSA |
| | | IASGVAVSKVLHLEGEVNKIKSALLSTNKAVVSLSNGVSVLTSKVLDLK |
| | | NYIDKQLLPIVNKQSCSISNIETVIEFQQKNNRLLEITREFSVNAGVTT |
| | | PVSTYMLTNSELLSLINDMPITNDQKKLMSNNVQIVRQQSYSIMSIIKE |
| | | EVLAYVVQLPLYGVIDTPCWKLHTSPLCTTNTKEGSNICLTRTDRGWYC |
| | | DNAGSVSFFPQAETCKVQSNRVFCDTMNSLTLPSEVNLCNVDIFNPKYD |
| | | CKIMTSKTDVSSSVITSLGAIVSCYGKTKCTASNKNRGIIKTFSNGCDY |
| | | VSNKGVDTVSVGNTLYYVNKQEGKSLYVKGEPIINFYDPLVFPSDEFDA |
| | | SISQVNEKINQSLAFIRKSDELLHNVNAGKSTTN |
| 3 | RSV F protein (C-terminal | MELLILKANAITTILTAVTFCFASGQNITEEFYQSTCSAVSKGYLSALR |
| | truncation aa 1-513, derived | TGWYTSVITIELSNIKENKCNGTDAKVKLIKQELDKYKNAVTELQLLMQ |
| | from A2 strain) | STPATNNRARRELPRFMNYTLNNAKKTNVTLSKKRKRRFLGFLLGVGSA |
| | | IASGVAVSKVLHLEGEVNKIKSALLSTNKAVVSLSNGVSVLTSKVLDLK |
| | | NYIDKQLLPIVNKQSCSISNIETVIEFQQKNNRLLEITREFSVNAGVTT |
| | | PVSTYMLTNSELLSLINDMPITNDQKKLMSNNVQIVRQQSYSIMSIIKE |
| | | EVLAYVVQLPLYGVIDTPCWKLHTSPLCTTNTKEGSNICLTRTDRGWYC |
| | | DNAGSVSFFPQAETCKVQSNRVFCDTMNSLTLPSEVNLCNVDIFNPKYD |
| | | CKIMTSKTDVSSSVITSLGAIVSCYGKTKCTASNKNRGIIKTFSNGCDY |
| | | VSNKGVDTVSVGNTLYYVNKQEGKSLYVKGEPIINFYDPLVFPSDEFDA |
| | | SISQVNEKINQSLAFIRKSDELL |
| 4 | RSV F protein truncated for | QNITEEFYQSTCSAVSKGYLSALRTGWYTSVITIELSNIKENKCNGTDA |
| | fusion (derived from A2 | KVKLIKQELDKYKNAVTELQLLMQSTPATNNRARRELPRFMNYTLNNAK |
| | strain) | KTNVTLSKKRKRRFLGFLLGVGSAIASGVAVSKVLHLEGEVNKIKSALL |
| | | STNKAVVSLSNGVSVLTSKVLDLKNYIDKQLLPIVNKQSCSISNIETVI |
| | | EFQQKNNRLLEITREFSVNAGVTTPVSTYMLTNSELLSLINDMPITNDQ |
| | | KKLMSNNVQIVRQQSYSIMSIIKEEVLAYVVQLPLYGVIDTPCWKLHTS |
| | | PLCTTNTKEGSNICLTRTDRGWYCDNAGSVSFFPQAETCKVQSNRVFCD |
| | | TMNSLTLPSEVNLCNVDIFNPKYDCKIMTSKTDVSSSVITSLGAIVSCY |
| | | GKTKCTASNKNRGIIKTFSNGCDYVSNKGVDTVSVGNTLYYVNKQEGKS |
| | | LYVKGEPIINFYDPLVFPSDEFDASISQVNEKINQSLAFIRKSDELL |
| 5 | RSV F1 protein (derived from | FLGFLLGVGSAIASGVAVSKVLHLEGEVNKIKSALLSTNKAVVSLSNGV |
| | A2 strain) (aa. 137-524) | SVLTSKVLDLKNYIDKQLLPIVNKQSCSISNIETVIEFQQKNNRLLEIT |
| | | REFSVNAGVTTPVSTYMLTNSELLSLINDMPITNDQKKLMSNNVQIVRQ |
| | | QSYSIMSIIKEEVLAYVVQLPLYGVIDTPCWKLHTSPLCTTNTKEGSNI |
| | | CLTRTDRGWYCDNAGSVSFFPQAETCKVQSNRVFCDTMNSLTLPSEVNL |
| | | CNVDIFNPKYDCKIMTSKTDVSSSVITSLGAIVSCYGKTKCTASNKNRG |
| | | REFSVNAGVTTPLSTYMLTNSELLSLINDMPITNDQKKLMSSNVQIVRQ |
| | | QSYSIMSIIKEEVLAYVVQLPIYGVIDTPCWKLHTSPLCTTNIKEGSNI |
| | | CLTRTDRGWYCDNAGSVSFFPQADTCKVQSNRVFCDTMNSLTLPSEVSL |
| | | CNTDIFNSKYDCKIMTSKTDISSSVITSLGAIVSCYGKTKCTASNKNRG |
| | | IIKTFSNGCDYVSNKGVDTVSVGNTLYYVNKLEGKNLYVKGEPIINYYD |
| | | PLVFPSDEFDASISQVNEKINQSLAFIRRSDELLHNVNTGKSTTNIIKT |
| | | FSNGCDYVSNKGVDTVSVGNTLYYVNKQEGKSLYVKGEPIINFYDPLVF |
| | | PSDEFDASISQVNEKINQSLAFIRKSDELLHNVNAGKSTTN |
| 6 | RSV F1 protein (derived from | FLGFLLGVGSAIASGVAVSKVLHLEGEVNKIKSALLSTNKAVVSLSNGV |
| | A2 strain) (aa. 137-513) | SVLTSKVLDLKNYIDKQLLPIVNKQSCSISNIETVIEFQQKNNRLLEIT |
| | | REFSVNAGVTTPVSTYMLTNSELLSLINDMPITNDQKKLMSNNVQIVRQ |
| | | QSYSIMSIIKEEVLAYVVQLPLYGVIDTPCWKLHTSPLCTTNTKEGSNI |
| | | CLTRTDRGWYCDNAGSVSFFPQAETCKVQSNRVFCDTMNSLTLPSEVNL |
| | | CNVDIFNPKYDCKIMTSKTDVSSSVITSLGAIVSCYGKTKCTASNKNRG |
| | | IIKTFSNGCDYVSNKGVDTVSVGNTLYYVNKQEGKSLYVKGEPIINFYD |
| | | PLVFPSDEFDASISQVNEKINQSLAFIRKSDELL |
| 7 | RSV F protein from 18537 | MELLIHRSSAIFLTLAVNALYLTSSQNITEEFYQSTCSAVSRGYFSALR |
| | strain (Swiss Prot P13843) | TGWYTSVITIELSNIKETKCNGTDTKVKLIKQELDKYKNAVTELQLLMQ |
| | | NTPAANNRARREAPQYMNYTINTTKNLNVSISKKRKRRFLGFLLGVGSA |
| | | IASGIAVSKVLHLEGEVNKIKNALLSTNKAVVSLSNGVSVLTSKVLDLK |
| | | NYINNRLLPIVNQQSCRISNIETVIEFQQMNSRLLEITREFSVNAGVTT |
| | | PLSTYMLTNSELLSLINDMPITNDQKKLMSSNVQIVRQQSYSIMSIIKE |
| | | EVLAYVVQLPIYGVIDTPCWKLHTSPLCTTNIKEGSNICLTRTDRGWYC |
| | | DNAGSVSFFPQADTCKVQSNRVFCDTMNSLTLPSEVSLCNTDIFNSKYD |
| | | CKIMTSKTDISSSVITSLGAIVSCYGKTKCTASNKNRGIIKTFSNGCDY |
| | | VSNKGVDTVSVGNTLYYVNKLEGKNLYVKGEPIINYYDPLVFPSDEFDA |
| | | SISQVNEKINQSLAFIRRSDELLHNVNTGKSTTNIMITTIIIVIIVVLL |
| | | SLIAIGLLLYCKAKNTPVTLSKDQLSGINNIAFSK |
| 8 | RSV F protein from 18537 | MELLIHRSSAIFLTLAVNALYLTSSQNITEEFYQSTCSAVSRGYFSALR |
| | strain (Swiss Prot P13843) | TGWYTSVITIELSNIKETKCNGTDTKVKLIKQELDKYKNAVTELQLLMQ |
| | (C-terminal truncation, | NTPAANNRARREAPQYMNYTINTTKNLNVSISKKRKRRFLGFLLGVGSA |
| | a.a. 1-524) | IASGIAVSKVLHLEGEVNKIKNALLSTNKAVVSLSNGVSVLTSKVLDLK |
| | | NYINNRLLPIVNQQSCRISNIETVIEFQQMNSRLLEITREFSVNAGVTT |
| | | PLSTYMLTNSELLSLINDMPITNDQKKLMSSNVQIVRQQSYSIMSIIKE |
| | | EVLAYVVQLPIYGVIDTPCWKLHTSPLCTTNIKEGSNICLTRTDRGWYC |
| | | DNAGSVSFFPQADTCKVQSNRVFCDTMNSLTLPSEVSLCNTDIFNSKYD |
| | | CKIMTSKTDISSSVITSLGAIVSCYGKTKCTASNKNRGIIKTFSNGCDY |
| | | VSNKGVDTVSVGNTLYYVNKLEGKNLYVKGEPIINYYDPLVFPSDEFDA |
| | | SISQVNEKINQSLAFIRRSDELLHNVNTGKSTTN |
| 9 | RSV F protein from 18537 | MELLIHRSSAIFLTLAVNALYLTSSQNITEEFYQSTCSAVSRGYFSALR |
| | strain (Swiss Prot P13843) | TGWYTSVITIELSNIKETKCNGTDTKVKLIKQELDKYKNAVTELQLLMQ |
| | (C-terminal truncation, | NTPAANNRARREAPQYMNYTINTTKNLNVSISKKRKRRFLGFLLGVGSA |
| | a.a. 1-513) | IASGIAVSKVLHLEGEVNKIKNALLSTNKAVVSLSNGVSVLTSKVLDLK |
| | | NYINNRLLPIVNQQSCRISNIETVIEFQQMNSRLLEITREFSVNAGVTT |
| | | PLSTYMLTNSELLSLINDMPITNDQKKLMSSNVQIVRQQSYSIMSIIKE |
| | | EVLAYVVQLPIYGVIDTPCWKLHTSPLCTTNIKEGSNICLTRTDRGWYC |
| | | DNAGSVSFFPQADTCKVQSNRVFCDTMNSLTLPSEVSLCNTDIFNSKYD |
| | | CKIMTSKTDISSSVITSLGAIVSCYGKTKCTASNKNRGIIKTFSNGCDY |
| | | VSNKGVDTVSVGNTLYYVNKLEGKNLYVKGEPIINYYDPLVFPSDEFDA |
| | | SISQVNEKINQSLAFIRRSDELL |
| 10 | RSV F protein truncated for | QNITEEFYQSTCSAVSRGYFSALRTGWYTSVITIELSNIKETKCNGTDT |
| | fusion (derived from 18537 | KVKLIKQELDKYKNAVTELQLLMQNTPAANNRARREAPQYMNYTINTTK |
| | strain) | NLNVSISKKRKRRFLGFLLGVGSAIASGIAVSKVLHLEGEVNKIKNALL |
| | | STNKAVVSLSNGVSVLTSKVLDLKNYINNRLLPIVNQQSCRISNIETVI |
| | | EFQQMNSRLLEITREFSVNAGVTTPLSTYMLTNSELLSLINDMPITNDQ |
| | | KKLMSSNVQIVRQQSYSIMSIIKEEVLAYVVQLPIYGVIDTPCWKLHTS |
| | | PLCTTNIKEGSNICLTRTDRGWYCDNAGSVSFFPQADTCKVQSNRVFCD |
| | | TMNSLTLPSEVSLCNTDIFNSKYDCKIMTSKTDISSSVITSLGAIVSCY |
| | | GKTKCTASNKNRGIIKTFSNGCDYVSNKGVDTVSVGNTLYYVNKLEGKN |
| | | LYVKGEPIINYYDPLVFPSDEFDASISQVNEKINQSLAFIRRSDELL |
| 11 | RSV F1 protein (derived from | FLGFLLGVGSAIASGIAVSKVLHLEGEVNKIKNALLSTNKAVVSLSNGV |
| | 18537 strain) (aa. 137-524) | SVLTSKVLDLKNYINNRLLPIVNQQSCRISNIETVIEFQQMNSRLLEIT |
| | | REFSVNAGVTTPLSTYMLTNSELLSLINDMPITNDQKKLMSSNVQIVRQ |
| | | QSYSIMSIIKEEVLAYVVQLPIYGVIDTPCWKLHTSPLCTTNIKEGSNI |
| | | CLTRTDRGWYCDNAGSVSFFPQADTCKVQSNRVFCDTMNSLTLPSEVSL |
| | | CNTDIFNSKYDCKIMTSKTDISSSVITSLGAIVSCYGKTKCTASNKNRG |
| | | IIKTFSNGCDYVSNKGVDTVSVGNTLYYVNKLEGKNLYVKGEPIINYYD |
| | | PLVFPSDEFDASISQVNEKINQSLAFIRRSDELLHNVNTGKSTTN |
| 12 | RSV F1 protein (derived from | FLGFLLGVGSAIASGIAVSKVLHLEGEVNKIKNALLSTNKAVVSLSNGV |
| | 18537 strain; C-terminal | SVLTSKVLDLKNYINNRLLPIVNQQSCRISNIETVIEFQQMNSRLLEIT |
| | truncation)(aa. 137-513) | REFSVNAGVTTPLSTYMLTNSELLSLINDMPITNDQKKLMSSNVQIVRQ |
| | | QSYSIMSIIKEEVLAYVVQLPIYGVIDTPCWKLHTSPLCTTNIKEGSNI |
| | | CLTRTDRGWYCDNAGSVSFFPQADTCKVQSNRVFCDTMNSLTLPSEVSL |
| | | CNTDIFNSKYDCKIMTSKTDISSSVITSLGAIVSCYGKTKCTASNKNRG |
| | | IIKTFSNGCDYVSNKGVDTVSVGNTLYYVNKLEGKNLYVKGEPIINYYD |
| | | PLVFPSDEFDASISQVNEKINQSLAFIRRSDELL |
| 13 | Ferritin (H. pylori J99) | MLSKDIIKLLNEQVNKEMNSSNLYMSMSSWCYTHSLDGAGLFLFDHAAE |
| | (amino acids 1-167) | EYEHAKKLIIFLNENNVPVQLTSISAPEHKFEGLTQIFQKAYEHEQHIS |
| | | ESINNIVDHAIKSKDHATENFLQWYVAEQHEEEVLEKDILDKIELIGNE |
| | | NHGLYLADQYVKGIAKSRKS |
| 14 | Ferritin (H. pylori J99) | MLSKDIIKLLNEQVNKEMNSSNLYMSMSSWCYTHSLDGAGLFLFDHAAE |
| | fragment (lacking C-terminal | EYEHAKKLIIFLNENNVPVQLTSISAPEHKFEGLTQIFQKAYEHEQHIS |
| | serine residue) (amino acids | ESINNIVDHAIKSKDHATENFLQWYVAEQHEEEVLEKDILDKIELIGNE |
| | 1-166) | NHGLYLADQYVKGIAKSRK |
| 15 | Ferritin heavy chain (Homo | MTTASTSQVRQNYHQDSEAAINRQINLELYASYVYLSMSYYFDRDDVAL |
| | sapiens) 183 amino acids | KNFAKYFLHQSHEEREHAEKLMKLQNQRGGRIFLQDIKKPDCDDWESGL |
| | | NAMECALHLEKNVNQSLLELHKLATDKNDPHLCDFIETHYLNEQVKAIK |
| | | ELGDHVTNLRKMGAPESGLAEYLFDKHTLGDSDNES |
| 16 | Ferritin (H. pylori) truncated | DIIKLLNEQVNKEMQSSNLYMSMSSWCYTHSLDGAGLFLFDHAAEEYEH |
| | for fusion (amino acids 5-167 | AKKLIIFLNENNVPVQLISISAPEHKEEGLTQIFQKAYEHEQHISESIN |
| | of full length H. pylori | NIVDHAIKSKDHATEMELQWYVAEQHEEEVLETDILDKIELIGNENHGL |
| | ferritin) | YLADQYVKGIAKSRK |
| 17 | Ferritin (H. pylori) truncated | DIIKLLNEQVNKEMQSSNLYMSMSSWCYTHSLDGAGLFLFDHAAEEYEH |
| | for fusion (amino acids 5-166 | AKKLIIFLNENNVPVQLISISAPEHKEEGLTQIFQKAYEHEQHISESIN |
| | of full length H. pylori | NIVDHAIKSKDHATFNFLQWYVAEQHEEEVLFKDILDKIELIGNENHGL |
| | ferritin) | YLADQYVKGIAKSRK |
| 18 | Truncated F glycoprotein | QNITEEFYQSTCSAVSKGYLSALRTGWYTSVITIELSNIKENKCNGTDA |
| | linked to ferritin (1); | KVKLIKQELDKYKNAVTELQLLMQSTPATNNRARRELPRFMNYTLNNAK |
| | SGGSGSG linker | KTNVTLSKKRKRRFLGFLLGVGSAIASGVAVSKVLHLEGEVNKIKSALL |
| | | STNKAVVSLSNGVSVLTSKVLDLKNYIDKQLLPIVNKQSCSISNIETVI |
| | | EFQQKNNRLLEITREFSVNAGVTTPVSTYMLTNSELLSLINDMPITNDQ |
| | | KKLMSNNVQIVRQQSYSIMSIIKEEVLAYVVQLPLYGVIDTPCWKLHTS |
| | | PLCTTNTKEGSNICLTRTDRGWYCDNAGSVSFFPQAETCKVQSNRVFCD |
| | | TMNSLTLPSEVNLCNVDIFNPKYDCKIMTSKTDVSSSVITSLGAIVSCY |
| | | GKTKCTASNKNRGIIKTFSNGCDYVSNKGVDTVSVGNTLYYVNKQEGKS |
| | | LYVKGEPIINFYDPLVFPSDEFDASISQVNEKINQSLAFIRKSDELLSG |
| | | GSGSGDIIKLLNEQVNKEMQSSNLYMSMSSWCYTHSLDGAGLFLFDHAA |
| | | EEYEHAKKLIIFLNENNVPVQLTSISAPEHKZEGLTQIFQKAYEHEQHI |
| | | SESINNIVDHAIKSKDHATFNELQWYVAEOHEETEVLEKDLDKIELIGN |
| | | ENHGLYLADQYVKGIAKSRES |
| 19 | Truncated F glycoprotein | QNITEEFYQSTCSAVSKGYLSALRTGWYTSVITIELSNIKENKCNGTDA |
| | linked to ferritin (2); SGSGSG | KVKLIKQELDKYKNAVTELQLLMQSTPATNNRARRELPRFMNYTLNNAK |
| | linker | KTNVTLSKKRKRRFLGFLLGVGSAIASGVAVSKVLHLEGEVNKIKSALL |
| | | STNKAVVSLSNGVSVLTSKVLDLKNYIDKQLLPIVNKQSCSISNIETVI |
| | | EFQQKNNRLLEITREFSVNAGVTTPVSTYMLTNSELLSLINDMPITNDQ |
| | | KKLMSNNVQIVRQQSYSIMSIIKEEVLAYVVQLPLYGVIDTPCWKLHTS |
| | | PLCTTNTKEGSNICLTRTDRGWYCDNAGSVSFFPQAETCKVQSNRVFCD |
| | | TMNSLTLPSEVNLCNVDIFNPKYDCKIMTSKTDVSSSVITSLGAIVSCY |
| | | GKTKCTASNKNRGIIKTFSNGCDYVSNKGVDTVSVGNTLYYVNKQEGKS |
| | | LYVKGEPIINFYDPLVFPSDEFDASISQVNEKINQSLAFIRKSDELLSG |
| | | SGSGDIIKLLNEOVNKEMQSSNLYMSMSSWCYTHSLDGAGLFLFDHAAE |
| | | EYEHAKKLIIFLNENNVPVOLISISAPEHKFEGLTQIFQKAYEHEQEIS |
| | | ESINNIVDHAIKSKDHATENFLQWYVAEQHEEEVLFKDILDKIELIGNE |
| | | NHGLYLADQYVKGIAKSRKS |
| 20 | Truncated F glycoprotein | QNITEEFYQSTCSAVSKGYLSALRTGWYTSVITIELSNIKENKCNGTDA |
| | linked to ferritin (3); SGGSG | KVKLIKQELDKYKNAVTELQLLMQSTPATNNRARRELPRFMNYTLNNAK |
| | linker | KTNVTLSKKRKRRFLGFLLGVGSAIASGVAVSKVLHLEGEVNKIKSALL |
| | | STNKAVVSLSNGVSVLTSKVLDLKNYIDKQLLPIVNKQSCSISNIETVI |
| | | EFQQKNNRLLEITREFSVNAGVTTPVSTYMLTNSELLSLINDMPITNDQ |
| | | KKLMSNNVQIVRQQSYSIMSIIKEEVLAYVVQLPLYGVIDTPCWKLHTS |
| | | PLCTTNTKEGSNICLTRTDRGWYCDNAGSVSFFPQAETCKVQSNRVFCD |
| | | TMNSLTLPSEVNLCNVDIFNPKYDCKIMTSKTDVSSSVITSLGAIVSCY |
| | | GKTKCTASNKNRGIIKTFSNGCDYVSNKGVDTVSVGNTLYYVNKQEGKS |
| | | LYVKGEPIINFYDPLVFPSDEFDASISQVNEKINQSLAFIRKSDELLSG |
| | | GSGDIIKLLNEQVNKEMQSSNLYMSMSSWCYTHSLDGAGLFLFDHAAEE |
| | | YEHAKKLIIFLNENNVRVOLTSISAPEHKEEGLIQIEQKAYEHEQHISE |
| | | SINNIVDHAIKSKDHATENFLQWYVAEQHEEEVLFKDILDKIELIGNEN |
| | | HGLYLADQYVKGIAKSRKS |
| 21 | Truncated F glycoprotein | QNITEEFYQSTCSAVSKGYLSALRTGWYTSVITIELSNIKENKCNGTDA |
| | linked to ferritin (4); SGSG | KVKLIKQELDKYKNAVTELQLLMQSTPATNNRARRELPRFMNYTLNNAK |
| | linker | KTNVTLSKKRKRRFLGFLLGVGSAIASGVAVSKVLHLEGEVNKIKSALL |
| | | STNKAVVSLSNGVSVLTSKVLDLKNYIDKQLLPIVNKQSCSISNIETVI |
| | | EFQQKNNRLLEITREFSVNAGVTTPVSTYMLTNSELLSLINDMPITNDQ |
| | | KKLMSNNVQIVRQQSYSIMSIIKEEVLAYVVQLPLYGVIDTPCWKLHTS |
| | | PLCTTNTKEGSNICLTRTDRGWYCDNAGSVSFFPQAETCKVQSNRVFCD |
| | | TMNSLTLPSEVNLCNVDIFNPKYDCKIMTSKTDVSSSVITSLGAIVSCY |
| | | GKTKCTASNKNRGIIKTFSNGCDYVSNKGVDTVSVGNTLYYVNKQEGKS |
| | | LYVKGEPIINFYDPLVFPSDEFDASISQVNEKINQSLAFIRKSDELLSG |
| | | SGDIIKLLNEQVNKEMQSSNLYMSMSSWCYTHSLDGAGLFLFDHAAEEY |
| | | EHAKKLIIFLNENNVPVQLTSISAPEHKEEGLTQIFQKAYEHEQHISES |
| | | INNIVDHAIKSKDHATENFLQWYVAEQHEEEVLEKDILDKIELIGNENH |
| | | GLYLADQYVKGIAKSRKS |
| 22 | Truncated F glycoprotein | QNITEEFYQSTCSAVSKGYLSALRTGWYTSVITIELSNIKENKCNGTDA |
| | linked to sHSP20 (1); | KVKLIKQELDKYKNAVTELQLLMQSTPATNNRARRELPRFMNYTLNNAK |
| | SGSGSGSGSG linker | KTNVTLSKKRKRRFLGFLLGVGSAIASGVAVSKVLHLEGEVNKIKSALL |
| | | STNKAVVSLSNGVSVLTSKVLDLKNYIDKQLLPIVNKQSCSISNIETVI |
| | | EFQQKNNRLLEITREFSVNAGVTTPVSTYMLTNSELLSLINDMPITNDQ |
| | | KKLMSNNVQIVRQQSYSIMSIIKEEVLAYVVQLPLYGVIDTPCWKLHTS |
| | | PLCTTNTKEGSNICLTRTDRGWYCDNAGSVSFFPQAETCKVQSNRVFCD |
| | | TMNSLTLPSEVNLCNVDIFNPKYDCKIMTSKTDVSSSVITSLGAIVSCY |
| | | GKTKCTASNKNRGIIKTFSNGCDYVSNKGVDTVSVGNTLYYVNKQEGKS |
| | | LYVKGEPIINFYDPLVFPSDEFDASISQVNEKINQSLAFIRKSDELLSG |
| | | SGSGSGSGTGTIMIQSSTGIQISGKGEMPISIIEGEQHIKVIAWLPGVN |
| | | KEDIILNAVGDTLEIRAKRSPLMITESERIIYSEIPEEEEIYRTIKLPA |
| | | IVKEENASAKEENGVLSVILPKAESSIKKGINIE |
| 23 | Truncated F glycoprotein | QNITEEFYQSTCSAVSKGYLSALRTGWYTSVITIELSNIKENKCNGTDA |
| | linked to HSP (2); | KVKLIKQELDKYKNAVTELQLLMQSTPATNNRARRELPRFMNYTLNNAK |
| | SGSGSGSGS linker | KTNVTLSKKRKRRFLGFLLGVGSAIASGVAVSKVLHLEGEVNKIKSALL |
| | | STNKAVVSLSNGVSVLTSKVLDLKNYIDKQLLPIVNKQSCSISNIETVI |
| | | EFQQKNNRLLEITREFSVNAGVTTPVSTYMLTNSELLSLINDMPITNDQ |
| | | KKLMSNNVQIVRQQSYSIMSIIKEEVLAYVVQLPLYGVIDTPCWKLHTS |
| | | PLCTTNTKEGSNICLTRTDRGWYCDNAGSVSFFPQAETCKVQSNRVFCD |
| | | TMNSLTLPSEVNLCNVDIFNPKYDCKIMTSKTDVSSSVITSLGAIVSCY |
| | | GKTKCTASNKNRGIIKTFSNGCDYVSNKGVDTVSVGNTLYYVNKQEGKS |
| | | LYVKGEPIINFYDPLVFPSDEFDASISQVNEKINQSLAFIRKSDELLSG |
| | | SGSGSGSIGTTMIQSSTGIQISGKGFMPISIIEGDQHIKVIAWLPGVNK |
| | | EDIILNAVGDILEIRAKRSPLMITESERIIYSEIPEEEEIYRTIKLPAI |
| | | VKEENASAKFENGVLSVILPKAESSIKKGINIE |
| 24 | F glycoprotein (w/N-terminal | MELLILKANAITTILTAVTFCFASGQNITEEFYQSTCSAVSKGYLSALR |
| | sequence) linked to ferritin | TGWYTSVITIELSNIKENKCNGTDAKVKLIKQELDKYKNAVTELQLLMQ |
| | (1); SGGSGSG linker | STPATNNRARRELPRFMNYTLNNAKKTNVTLSKKRKRRFLGFLLGVGSA |
| | | IASGVAVSKVLHLEGEVNKIKSALLSTNKAVVSLSNGVSVLTSKVLDLK |
| | | NYIDKQLLPIVNKQSCSISNIETVIEFQQKNNRLLEITREFSVNAGVTT |
| | | PVSTYMLTNSELLSLINDMPITNDQKKLMSNNVQIVRQQSYSIMSIIKE |
| | | EVLAYVVQLPLYGVIDTPCWKLHTSPLCTTNTKEGSNICLTRTDRGWYC |
| | | DNAGSVSFFPQAETCKVQSNRVFCDTMNSLTLPSEVNLCNVDIFNPKYD |
| | | CKIMTSKTDVSSSVITSLGAIVSCYGKTKCTASNKNRGIIKTFSNGCDY |
| | | VSNKGVDTVSVGNTLYYVNKQEGKSLYVKGEPIINFYDPLVFPSDEEDA |
| | | SISQVNEKINQSLAFIRKSDELLSGGSGSGDIIKLLNEQVNKEMQSSNL |
| | | YMSMSSWCYTHSLDGAGLFLFDHAAEEYEHAKKLIIFLNENNVPVQLTS |
| | | ISAPEHKFEGLIQIFQKAYEHEQHISESINNIVDHAIKSKDHATFNFLQ |
| | | WYVAEQHEEEVLFKDILDKIELIGNENHGLYLADQYVKGIAKSRKS |
| 25 | F glycoprotein (w/N-terminal | MELLILKANAITTILTAVTFCFASGQNITEEFYQSTCSAVSKGYLSALR |
| | sequence) linked to ferritin | TGWYTSVITIELSNIKENKCNGTDAKVKLIKQELDKYKNAVTELQLLMQ |
| | (2); SGSGSG linker | STPATNNRARRELPRFMNYTLNNAKKTNVTLSKKRKRRFLGFLLGVGSA |
| | | IASGVAVSKVLHLEGEVNKIKSALLSTNKAVVSLSNGVSVLTSKVLDLK |
| | | NYIDKQLLPIVNKQSCSISNIETVIEFQQKNNRLLEITREFSVNAGVTT |
| | | PVSTYMLTNSELLSLINDMPITNDQKKLMSNNVQIVRQQSYSIMSIIKE |
| | | EVLAYVVQLPLYGVIDTPCWKLHTSPLCTTNTKEGSNICLTRTDRGWYC |
| | | DNAGSVSFFPQAETCKVQSNRVFCDTMNSLTLPSEVNLCNVDIFNPKYD |
| | | CKIMTSKTDVSSSVITSLGAIVSCYGKTKCTASNKNRGIIKTFSNGCDY |
| | | VSNKGVDTVSVGNTLYYVNKQEGKSLYVKGEPIINFYDPLVFPSDEEDA |
| | | SISQVNEKINQSLAFIRKSDELLSGSGSGDIIKLLNEQVNKEMQSSNLY |
| | | MSMSSWCYTHSLDGAGLFLFDHAAEEYEHAKKLIIFLNENNVPVQLTSI |
| | | SAPEHKFEGLTQIFQKAYEHEQHISESINNIVDHAIKSKDHATFNFLQW |
| | | YVAEQHEEEVLFKDILDKIELIGNENHGLYLADQYVKGIAKSRKS |
| 26 | F glycoprotein (w/N-terminal | MELLILKANAITTILTAVTFCFASGQNITEEFYQSTCSAVSKGYLSALR |
| | sequence) linked to ferritin | TGWYTSVITIELSNIKENKCNGTDAKVKLIKQELDKYKNAVTELQLLMQ |
| | (3); SGGSG linker | STPATNNRARRELPRFMNYTLNNAKKTNVTLSKKRKRRFLGFLLGVGSA |
| | | IASGVAVSKVLHLEGEVNKIKSALLSTNKAVVSLSNGVSVLTSKVLDLK |
| | | NYIDKQLLPIVNKQSCSISNIETVIEFQQKNNRLLEITREFSVNAGVTT |
| | | PVSTYMLTNSELLSLINDMPITNDQKKLMSNNVQIVRQQSYSIMSIIKE |
| | | EVLAYVVQLPLYGVIDTPCWKLHTSPLCTTNTKEGSNICLTRTDRGWYC |
| | | DNAGSVSFFPQAETCKVQSNRVFCDTMNSLTLPSEVNLCNVDIFNPKYD |
| | | CKIMTSKTDVSSSVITSLGAIVSCYGKTKCTASNKNRGIIKTFSNGCDY |
| | | VSNKGVDTVSVGNTLYYVNKQEGKSLYVKGEPIINFYDPLVFPSDEEDA |
| | | SISQVNEKINQSLAFIRKSDELLSGGSGDIIKLLNEQVNKEMOSSNLYM |
| | | SMSSWCYTHSLDGAGLFLFDHAAEEYEHAKKLIIFLNENNVPVQLTSIS |
| | | APEHKFEGLTQIFQKAYEHEQHISESINNIVDHAIKSKDHATFNFLQWY |
| | | VAEQHEEEVLFKDILDKIELIGNENHGLYLADQYVKGIAKSRKS |
| 27 | F glycoprotein (w/N-terminal | MELLILKANAITTILTAVTFCFASGQNITEEFYQSTCSAVSKGYLSALR |
| | sequence) linked to ferritin | TGWYTSVITIELSNIKENKCNGTDAKVKLIKQELDKYKNAVTELQLLMQ |
| | (4); SGSG linker | STPATNNRARRELPRFMNYTLNNAKKTNVTLSKKRKRRFLGFLLGVGSA |
| | | IASGVAVSKVLHLEGEVNKIKSALLSTNKAVVSLSNGVSVLTSKVLDLK |
| | | NYIDKQLLPIVNKQSCSISNIETVIEFQQKNNRLLEITREFSVNAGVTT |
| | | PVSTYMLTNSELLSLINDMPITNDQKKLMSNNVQIVRQQSYSIMSIIKE |
| | | EVLAYVVQLPLYGVIDTPCWKLHTSPLCTTNTKEGSNICLTRTDRGWYC |
| | | DNAGSVSFFPQAETCKVQSNRVFCDTMNSLTLPSEVNLCNVDIFNPKYD |
| | | CKIMTSKTDVSSSVITSLGAIVSCYGKTKCTASNKNRGIIKTFSNGCDY |
| | | VSNKGVDTVSVGNTLYYVNKQEGKSLYVKGEPIINFYDPLVFPSDEFDA |
| | | SISQVNEKINQSLAFIRKSDELLSGSGDIIKLLNEQVNKEMQSSNLYMS |
| | | MSSWCYTHSLDGAGLFLFDHAAEEYEHAKKLIIFLNENNVPVQLTSISA |
| | | PEHKFEGLTQIFQKAYEHEQHISESINMIVDHAIKSKDHATFNFLQWYV |
| | | AEQHEEEVLFKDILDKIELIGNENHGLYLADQYVKGIAKSRKS |
| 28 | F glycoprotein (w/N-terminal | MELLILKANAITTILTAVTFCFASGQNITEEFYQSTCSAVSKGYLSALR |
| | sequence) linked to sHSP20 | TGWYTSVITIELSNIKENKCNGTDAKVKLIKQELDKYKNAVTELQLLMQ |
| | (1); SGSGSGSGSG linker | STPATNNRARRELPRFMNYTLNNAKKTNVTLSKKRKRRFLGFLLGVGSA |
| | | IASGVAVSKVLHLEGEVNKIKSALLSTNKAVVSLSNGVSVLTSKVLDLK |
| | | NYIDKQLLPIVNKQSCSISNIETVIEFQQKNNRLLEITREFSVNAGVTT |
| | | PVSTYMLTNSELLSLINDMPITNDQKKLMSNNVQIVRQQSYSIMSIIKE |
| | | EVLAYVVQLPLYGVIDTPCWKLHTSPLCTTNTKEGSNICLTRTDRGWYC |
| | | DNAGSVSFFPQAETCKVQSNRVFCDTMNSLTLPSEVNLCNVDIFNPKYD |
| | | CKIMTSKTDVSSSVITSLGAIVSCYGKTKCTASNKNRGIIKTFSNGCDY |
| | | VSNKGVDTVSVGNTLYYVNKQEGKSLYVKGEPIINFYDPLVFPSDEFDA |
| | | SISQVNEKINQSLAFIRKSDELLSGSGSGSGSGTGTTMIQSSTGIQISG |
| | | KGFMPISIIEGDQHIKVIAWLPGVNKEDIILNAVGDTLEIRAKRSPLMI |
| | | TESERIIYSEIPEEEEIYRTIKLPATVKEENASAKFENGVLSVILPKAE |
| | | SSIKKGINIE |
| 29 | F glycoprotein (w/N-terminal | MELLILKANAITTILTAVTFCFASGQNITEEFYQSTCSAVSKGYLSALR |
| | sequence) linked to HSP (2); | TGWYTSVITIELSNIKENKCNGTDAKVKLIKQELDKYKNAVTELQLLMQ |
| | SGSGSGSGS linker | STPATNNRARRELPRFMNYTLNNAKKTNVTLSKKRKRRFLGFLLGVGSA |
| | | IASGVAVSKVLHLEGEVNKIKSALLSTNKAVVSLSNGVSVLTSKVLDLK |
| | | NYIDKQLLPIVNKQSCSISNIETVIEFQQKNNRLLEITREFSVNAGVTT |
| | | PVSTYMLTNSELLSLINDMPITNDQKKLMSNNVQIVRQQSYSIMSIIKE |
| | | EVLAYVVQLPLYGVIDTPCWKLHTSPLCTTNTKEGSNICLTRTDRGWYC |
| | | DNAGSVSFFPQAETCKVQSNRVFCDTMNSLTLPSEVNLCNVDIFNPKYD |
| | | CKIMTSKTDVSSSVITSLGAIVSCYGKTKCTASNKNRGIIKTFSNGCDY |
| | | VSNKGVDTVSVGNTLYYVNKQEGKSLYVKGEPIINFYDPLVFPSDEFDA |
| | | SISQVNEKINQSLAFIRKSDELLSGSGSGSGSTGTTMIQSSTGIQISGK |
| | | GFMPISIIEGDQHIKVIAWLPGVNKEDIILNAVGDTLEIRAKRSPLMIT |
| | | ESERIIYSEIPEEEEIYRTIKLPATVKEENASAKFENGVLSVILPKAES |
| | | SIKKGINIE |
| 30 | Leader-RSVF-linker-HypF | MPMGSLQPLATLYLLGMLVASCLGMELLILKANAITTILTAVTFCFASG |
| | (1); SGGSGSG linker | QNITEEFYQSTCSAVSKGYLSALRTGWYTSVITIELSNIKENKCNGTDA |
| | | KVKLIKQELDKYKNAVTELQLLMQSTPATNNRARRELPRFMNYTLNNAK |
| | | KTNVTLSKKRKRRFLGFLLGVGSAIASGVAVSKVLHLEGEVNKIKSALL |
| | | STNKAVVSLSNGVSVLTSKVLDLKNYIDKQLLPIVNKQSCSISNIETVI |
| | | EFQQKNNRLLEITREFSVNAGVTTPVSTYMLTNSELLSLINDMPITNDQ |
| | | KKLMSNNVQIVRQQSYSIMSIIKEEVLAYVVQLPLYGVIDTPCWKLHTS |
| | | PLCTTNTKEGSNICLTRTDRGWYCDNAGSVSFFPQAETCKVQSNRVFCD |
| | | TMNSLTLPSEVNLCNVDIFNPKYDCKIMTSKTDVSSSVITSLGAIVSCY |
| | | GKTKCTASNKNRGIIKTFSNGCDYVSNKGVDTVSVGNTLYYVNKQEGKS |
| | | LYVKGEPIINFYDPLVFPSDEFDASISQVNEKINQSLAFIRKSDELLSG |
| | | GSGSGDIIKLLNEQVNKEMQSSNLYMSMSSWCYTHSLDGAGLFLFDHAA |
| | | EEYEHAKKLIIFLNENNVPVQLTSISAPEHKFEGLTQIFQKAYEHEQHI |
| | | SESINNIVDHAIKSKDHATFNFLQWYVAEQHEEEVLFKDILDKIELIGN |
| | | ENHGLYLADQYVKGIAKSRK |
| 31 | Leader-RSVF-linker-HypF | MPMGSLQPLATLYLLGMLVASCLGMELLILKANAITTILTAVTFCFASG |
| | (2); SGSGSG linker | QNITEEFYQSTCSAVSKGYLSALRTGWYTSVITIELSNIKENKCNGTDA |
| | | KVKLIKQELDKYKNAVTELQLLMQSTPATNNRARRELPRFMNYTLNNAK |
| | | KTNVTLSKKRKRRFLGFLLGVGSAIASGVAVSKVLHLEGEVNKIKSALL |
| | | STNKAVVSLSNGVSVLTSKVLDLKNYIDKQLLPIVNKQSCSISNIETVI |
| | | EFQQKNNRLLEITREFSVNAGVTTPVSTYMLTNSELLSLINDMPITNDQ |
| | | KKLMSNNVQIVRQQSYSIMSIIKEEVLAYVVQLPLYGVIDTPCWKLHTS |
| | | PLCTTNTKEGSNICLTRTDRGWYCDNAGSVSFFPQAETCKVQSNRVFCD |
| | | TMNSLTLPSEVNLCNVDIFNPKYDCKIMTSKTDVSSSVITSLGAIVSCY |
| | | GKTKCTASNKNRGIIKTFSNGCDYVSNKGVDTVSVGNTLYYVNKQEGKS |
| | | LYVKGEPIINFYDPLVFPSDEFDASISQVNEKINQSLAFIRKSDELLSG |
| | | SGSGDIIKLLNEQVNKEMQSSNLYMSMSSWCYTHSLDGAGLFLFDHAAE |
| | | EYEHAKKLIIFLNENNVPVQLTSISAPEHKFEGLTQIFQKAYEHEQHIS |
| | | ESINNIVDHAIKSKDHATFNFLQWYVAEQHEEEVLFKDILDKIELIGNE |
| | | NHGLYLADQYVKGIAKSRK |
| 32 | Leader-RSVF-linker-HypF | MPMGSLQPLATLYLLGMLVASCLGMELLILKANAITTILTAVTFCFASG |
| | (3); SGGSG linker | QNITEEFYQSTCSAVSKGYLSALRTGWYTSVITIELSNIKENKCNGTDA |
| | | KVKLIKQELDKYKNAVTELQLLMQSTPATNNRARRELPRFMNYTLNNAK |
| | | KTNVTLSKKRKRRFLGFLLGVGSAIASGVAVSKVLHLEGEVNKIKSALL |
| | | STNKAVVSLSNGVSVLTSKVLDLKNYIDKQLLPIVNKQSCSISNIETVI |
| | | EFQQKNNRLLEITREFSVNAGVTTPVSTYMLTNSELLSLINDMPITNDQ |
| | | KKLMSNNVQIVRQQSYSIMSIIKEEVLAYVVQLPLYGVIDTPCWKLHTS |
| | | PLCTTNTKEGSNICLTRTDRGWYCDNAGSVSFFPQAETCKVQSNRVFCD |
| | | TMNSLTLPSEVNLCNVDIFNPKYDCKIMTSKTDVSSSVITSLGAIVSCY |
| | | GKTKCTASNKNRGIIKTFSNGCDYVSNKGVDTVSVGNTLYYVNKQEGKS |
| | | LYVKGEPIINFYDPLVFPSDEFDASISQVNEKINQSLAFIRKSDELLSG |
| | | GSGDIIKLLNEQVNKEMQSSNLYMSMSSWCYTHSLDGAGLFLFDHAAEE |
| | | YEHAKKLIIFLNENNVPVQLTSISAPEHKFEGLTQIFQKAYEHEQHISE |
| | | SINNIVDHAIKSKDHATFNFLQWYVAEQHEEEVLFKDILDKIELIGNEN |
| | | HGLYLADQYVKGIAKSRK |
| 33 | Leader-full length RSVF- | MPMGSLQPLATLYLLGMLVASCLGMELLILKANAITTILTAVTFCFASG |
| | linker-HypF (4); SGSG linker | QNITEEFYQSTCSAVSKGYLSALRTGWYTSVITIELSNIKENKCNGTDA |
| | | KVKLIKQELDKYKNAVTELQLLMQSTPATNNRARRELPRFMNYTLNNAK |
| | | KTNVTLSKKRKRRFLGFLLGVGSAIASGVAVSKVLHLEGEVNKIKSALL |
| | | STNKAVVSLSNGVSVLTSKVLDLKNYIDKQLLPIVNKQSCSISNIETVI |
| | | EFQQKNNRLLEITREFSVNAGVTTPVSTYMLTNSELLSLINDMPITNDQ |
| | | KKLMSNNVQIVRQQSYSIMSIIKEEVLAYVVQLPLYGVIDTPCWKLHTS |
| | | PLCTTNTKEGSNICLTRTDRGWYCDNAGSVSFFPQAETCKVQSNRVFCD |
| | | TMNSLTLPSEVNLCNVDIFNPKYDCKIMTSKTDVSSSVITSLGAIVSCY |
| | | GKTKCTASNKNRGIIKTFSNGCDYVSNKGVDTVSVGNTLYYVNKQEGKS |
| | | LYVKGEPIINFYDPLVFPSDEFDASISQVNEKINQSLAFIRKSDELLSG |
| | | SGDIIKLLNEQVNKEMQSSNLYMSMSSWCYTHSLDGAGLFLFDHAAEEY |
| | | EHAKKLIIFLNENNVPVQLTSISAPEHKFEGLTQIFQKAYEHEQHISES |
| | | INNIVDHAIKSKDHATFNFLQWYVAEQHEEEVLFKDILDKIELIGNENH |
| | | GLYLADQYVKGIAKSRK |
| 34 | Leader-RSVF-linker-sHSP20 | MPMGSLQPLATLYLLGMLVASCLGMELLILKANAITTILTAVTFCFASG |
| | (1); SGSGSGSGSG linker | QNITEEFYQSTCSAVSKGYLSALRTGWYTSVITIELSNIKENKCNGTDA |
| | | KVKLIKQELDKYKNAVTELQLLMQSTPATNNRARRELPRFMNYTLNNAK |
| | | KTNVTLSKKRKRRFLGFLLGVGSAIASGVAVSKVLHLEGEVNKIKSALL |
| | | STNKAVVSLSNGVSVLTSKVLDLKNYIDKQLLPIVNKQSCSISNIETVI |
| | | EFQQKNNRLLEITREFSVNAGVTTPVSTYMLTNSELLSLINDMPITNDQ |
| | | KKLMSNNVQIVRQQSYSIMSIIKEEVLAYVVQLPLYGVIDTPCWKLHTS |
| | | PLCTTNTKEGSNICLTRTDRGWYCDNAGSVSFFPQAETCKVQSNRVFCD |
| | | TMNSLTLPSEVNLCNVDIFNPKYDCKIMTSKTDVSSSVITSLGAIVSCY |
| | | GKTKCTASNKNRGIIKTFSNGCDYVSNKGVDTVSVGNTLYYVNKQEGKS |
| | | LYVKGEPIINFYDPLVFPSDEFDASISQVNEKINQSLAFIRKSDELLSG |
| | | SGSGSGSGTGTTMIQSSTGIQISGKGFMPISIIEGDQHIKVIAWLPGVN |
| | | KEDIILNAVGDTLEIRAKRSPLMITESERIIYSEIPEEEEIYRTIKLPA |
| | | TVKEENASAKFENGVLSVILPKAESSIKKGINIE |
| 35 | Leader-RSVF-linker-sHSP20 | MPMGSLQPLATLYLLGMLVASCLGMELLILKANAITTILTAVTFCFASG |
| | (2); SGSGSGSGS linker | QNITEEFYQSTCSAVSKGYLSALRTGWYTSVITIELSNIKENKCNGTDA |
| | | KVKLIKQELDKYKNAVTELQLLMQSTPATNNRARRELPRFMNYTLNNAK |
| | | KTNVTLSKKRKRRFLGFLLGVGSAIASGVAVSKVLHLEGEVNKIKSALL |
| | | STNKAVVSLSNGVSVLTSKVLDLKNYIDKQLLPIVNKQSCSISNIETVI |
| | | EFQQKNNRLLEITREFSVNAGVTTPVSTYMLTNSELLSLINDMPITNDQ |
| | | KKLMSNNVQIVRQQSYSIMSIIKEEVLAYVVQLPLYGVIDTPCWKLHTS |
| | | PLCTTNTKEGSNICLTRTDRGWYCDNAGSVSFFPQAETCKVQSNRVFCD |
| | | TMNSLTLPSEVNLCNVDIFNPKYDCKIMTSKTDVSSSVITSLGAIVSCY |
| | | GKTKCTASNKNRGIIKTFSNGCDYVSNKGVDTVSVGNTLYYVNKQEGKS |
| | | LYVKGEPIINFYDPLVFPSDEFDASISQVNEKINQSLAFIRKSDELLSG |
| | | SGSGSGSTGTTMIQSSTGIQISGKGFMPISIIEGDQHIKVIAWLPGVNK |
| | | EDIILNAVGDTLEIRAKRSPLMITESERIIYSEIPEEEEIYRTIKLPAT |
| | | VKEENASAKFENGVLSVILPKAESSIKKGINIE |
| 36 | sHSP20 full length | MFGRDPFDSLFERMFKEFFATPMTGTTMIQSSTGIQISGKGFMPISIIE |
| | (NP_247258.1) | GDQHIKVIAWLPGVNKEDIILNAVGDTLEIRAKRSPLMITESERIIYSE |
| | | IPEEEEIYRTIKLPATVKEENASAKFENGVLSVILPKAESSIKKGINIE |
| 37 | sHSP20 truncated for fusion | TGTTMIQSSTGIQISGKGFMPISIIEGDQHIKVIAWLPGVNKEDIILNA |
| | (amino acids 24-147 of full | VGDTLEIRAKRSPLMITESERIIYSEIPEEEEIYRTIKLPATVKEENAS |
| | length sHSP20) | AKFENGVLSVILPKAESSIKKGINIE |
| 38 | HSP60 (human) | MLRLPTVFRQMRPVSRVLAPHLTRAYAKDVKFGADARALMLQGVDLLAD |
| | (NP_002147.2) | AVAVTMGPKGRTVIIEQSWGSPKVTKDGVTVAKSIDLKDKYKNIGAKLV |
| | | QDVANNTNEEAGDGTTTATVLARSIAKEGFEKISKGANPVEIRRGVMLA |
| | | VDAVIAELKKQSKPVTTPEEIAQVATISANGDKEIGNIISDAMKKVGRK |
| | | GVITVKDGKTLNDELEIIEGMKFDRGYISPYFINTSKGQKCEFQDAYVL |
| | | LSEKKISSIQSIVPALEIANAHRKPLVIIAEDVDGEALSTLVLNRLKVG |
| | | LQVVAVKAPGFGDNRKNQLKDMAIATGGAVFGEEGLTLNLEDVQPHDLG |
| | | KVGEVIVTKDDAMLLKGKGDKAQIEKRIQEIIEQLDVTTSEYEKEKLNE |
| | | RLAKLSDGVAVLKVGGTSDVEVNEKKDRVTDALNATRAAVEEGIVLGGG |
| | | CALLRCIPALDSLTPANEDQKIGIEIIKRTLKIPAMTIAKNAGVEGSLI |
| | | VEKIMQSSSEVGYDAMAGDFVNMVEKGIIDPTKVVRTALLDAAGVASLL |
| | | TTAEVVVTEIPKEEKDPGMGAMGGMGGGMGGGMF |
| 39 | HSP70 (human) | MSVVGIDLGFQSCYVAVARAGGIETIANEYSDRCTPACISFGPKNRSIG |
| | (NP_002145.3) | AAAKSQVISNAKNTVQGFKRFHGRAFSDPFVEAEKSNLAYDIVQLPTGL |
| | | TGIKVTYMEEERNFTTEQVTAMLLSKLKETAESVLKKPVVDCVVSVPCF |
| | | YTDAERRSVMDATQIAGLNCLRLMNETTAVALAYGIYKQDLPALEEKPR |
| | | NVVFVDMGHSAYQVSVCAFNRGKLKVLATAFDTTLGGRKFDEVLVNHFC |
| | | EEFGKKYKLDIKSKIRALLRLSQECEKLKKLMSANASDLPLSIECFMND |
| | | VDVSGTMNRGKFLEMCNDLLARVEPPLRSVLEQTKLKKEDIYAVEIVGG |
| | | ATRIPAVKEKISKFFGKELSTTLNADEAVTRGCALQCAILSPAFKVREF |
| | | SITDVVPYPISLRWNSPAEEGSSDCEVFSKNHAAPFSKVLTFYRKEPFT |
| | | LEAYYSSPQDLPYPDPAIAQFSVQKVTPQSDGSSSKVKVKVRVNVHGIF |
| | | SVSSASLVEVHKSEENEEPMETDQNAKEEEKMQVDQEEPHVEEQQQQTP |
| | | AENKAESEEMETSQAGSKDKKMDQPPQAKKAKVKTSTVDLPIENQLLWQ |
| | | IDREMLNLYIENEGKMIMQDKLEKERNDAKNAVEEYVYEMRDKLSGEYE |
| | | KFVSEDDRNSFTLKLEDTENWLYEDGEDQPKQVYVDKLAELKNLGQPIK |
| | | IRFQESEERPKLFEELGKQIQQYMKIISSFKNKEDQYDHLDAADMTKVE |
| | | KSTNEAMEWMNNKLNLQNKQSLTMDPVVKSKEIEAKIKELTSTCSPIIS |
| | | KPKPKVEPPKEEQKNAEQNGPVDGQGDNPGPQAAEQGTDTAVPSDSDKK |
| | | LPEMDID |
| 40 | HSP90 alpha isoform 1 | MPPCSGGDGSTPPGPSLRDRDCPAQSAEYPRDRLDPRPGSPSEASSPPF |
| | (human) (NP_001017963.2) | LRSRAPVNWYQEKAQVFLWHLMVSGSTTLLCLWKQPFHVSAFPVTASLA |
| | | FRQSQGAGQHLYKDLQPFILLRLLMPEETQTQDQPMEEEEVETFAFQAE |
| | | IAQLMSLIINTFYSNKEIFLRELISNSSDALDKIRYESLTDPSKLDSGK |
| | | ELHINLIPNKQDRTLTIVDTGIGMTKADLINNLGTIAKSGTKAFMEALQ |
| | | AGADISMIGQFGVGFYSAYLVAEKVTVITKHNDDEQYAWESSAGGSFTV |
| | | RTDTGEPMGRGTKVILHLKEDQTEYLEERRIKEIVKKHSQFIGYPITLF |
| | | VEKERDKEVSDDEAEEKEDKEEEKEKEEKESEDKPEIEDVGSDEEEEKK |
| | | DGDKKKKKKIKEKYIDQEELNKTKPIWTRNPDDITNEEYGEFYKSLTND |
| | | WEDHLAVKHFSVEGQLEFRALLFVPRRAPFDLFENRKKKNNIKLYVRRV |
| | | FIMDNCEELIPEYLNFIRGVVDSEDLPLNISREMLQQSKILKVIRKNLV |
| | | KKCLELFTELAEDKENYKKFYEQFSKNIKLGIHEDSQNRKKLSELLRYY |
| | | TSASGDEMVSLKDYCTRMKENQKHIYYITGETKDQVANSAFVERLRKHG |
| | | LEVIYMIEPIDEYCVQQLKEFEGKTLVSVTKEGLELPEDEEEKKKQEEK |
| | | KTKFENLCKIMKDILEKKVEKVVVSNRLVTSPCCIVTSTYGWTANMERI |
| | | MKAQALRDNSTMGYMAAKKHLEINPDHSIIETLRQKAEADKNDKSVKDL |
| | | VILLYETALLSSGFSLEDPQTHANRIYRMIKLGLGIDEDDPTADDTSAA |
| | | VTEEMPPLEGDDDTSRMEEVD |
| 41 | HSP90 alpha isoform 2 | MPEETQTQDQPMEEEEVETFAFQAEIAQLMSLIINTFYSNKEIFLRELI |
| | (human) (NP_005339.3) | SNSSDALDKIRYESLTDPSKLDSGKELHINLIPNKQDRTLTIVDTGIGM |
| | | TKADLINNLGTIAKSGTKAFMEALQAGADISMIGQFGVGFYSAYLVAEK |
| | | VTVITKHNDDEQYAWESSAGGSFTVRTDTGEPMGRGTKVILHLKEDQTE |
| | | YLEERRIKEIVKKHSQFIGYPITLFVEKERDKEVSDDEAEEKEDKEEEK |
| | | EKEEKESEDKPEIEDVGSDEEEEKKDGDKKKKKKIKEKYIDQEELNKTK |
| | | PIWTRNPDDITNEEYGEFYKSLTNDWEDHLAVKHFSVEGQLEFRALLFV |
| | | PRRAPFDLFENRKKKNNIKLYVRRVFIMDNCEELIPEYLNFIRGVVDSE |
| | | DLPLNISREMLQQSKILKVIRKNLVKKCLELFTELAEDKENYKKFYEQF |
| | | SKNIKLGIHEDSQNRKKLSELLRYYTSASGDEMVSLKDYCTRMKENQKH |
| | | IYYITGETKDQVANSAFVERLRKHGLEVIYMIEPIDEYCVQQLKEFEGK |
| | | TLVSVTKEGLELPEDEEEKKKQEEKKTKFENLCKIMKDILEKKVEKVVV |
| | | SNRLVTSPCCIVTSTYGWTANMERIMKAQALRDNSTMGYMAAKKHLEIN |
| | | PDHSIIETLRQKAEADKNDKSVKDLVILLYETALLSSGFSLEDPQTHAN |
| | | RIYRMIKLGLGIDEDDPTADDTSAAVTEEMPPLEGDDDTSRMEEVD |
| 42 | HSP100 (human) | MPSCGACTCGAAAVRLITSSLASAQRGISGGRIHMSVLGRLGTFETQIL |
| | (NP_006651.2) | QRAPLRSFTETPAYFASKDGISKDGSGDGNKKSASEGSSKKSGSGNSGK |
| | | GGNQLRCPKCGDLCTHVETFVSSTRFVKCEKCHHFFVVLSEADSKKSII |
| | | KEPESAAEAVKLAFQQKPPPPPKKIYNYLDKYVVGQSFAKKVLSVAVYN |
| | | HYKRIYNNIPANLRQQAEVEKQTSLTPRELEIRRREDEYRFTKLLQIAG |
| | | ISPHGNALGASMQQQVNQQIPQEKRGGEVLDSSHDDIKLEKSNILLLGP |
| | | TGSGKTLLAQTLAKCLDVPFAICDCTTLTQAGYVGEDIESVIAKLLQDA |
| | | NYNVEKAQQGIVFLDEVDKIGSVPGIHQLRDVGGEGVQQGLLKLLEGTI |
| | | VNVPEKNSRKLRGETVQVDTTNILFVASGAFNGLDRIISRRKNEKYLGF |
| | | GTPSNLGKGRRAAAAADLANRSGESNTHQDIEEKDRLLRHVEARDLIEF |
| | | GMIPEFVGRLPVVVPLHSLDEKTLVQILTEPRNAVIPQYQALFSMDKCE |
| | | LNVTEDALKAIARLALERKTGARGLRSIMEKLLLEPMFEVPNSDIVCVE |
| | | VDKEVVEGKKEPGYIRAPTKESSEEEYDSGVEEEGWPRQADAANS |
| 43 | Linker | SGSG |
| 44 | Linker | NGTGGSG |
| 45 | Linker | SGGSG |
| 46 | Linker | GGSGSG |
| 47 | Linker | SGSGSG |
| 48 | Linker | SGGSGSG |
| 49 | Linker | SGSGSGSGS |
| 50 | Linker | SGSGSGSGSG |
| 51 | N-terminal leader (from | MPMGSLQPLATLYLLGMLVASCLG |
| | human CD5) | |
| 52 | N-terminal leader | METDTLLLWVLLLWVPGSTG |
| 53 | N-terminal leader | MDSYLLMWGLLTFIMVPGCQA |
| 54 | N-terminal F protein sequence | MELLILKANAITTILTAVTFCFASG |

We claim:

1. A nanoparticle comprising Respiratory syncytial virus (RSV) F protein, or fragment thereof, and a self-assembling molecule, wherein the self-assembling molecule forms a polymeric assembly that captures the F protein or fragment thereof in a meta-stable pre-fusion conformation, thereby forming the nanoparticle.

2. The nanoparticle of claim 1, wherein the F protein comprises (a) an F1 and F2 heterodimer, (b) an ectodomain, (c) a heptad-repeat A domain (HRA) and a heptad-repeat C domain (HRC), (d) an HRA domain, an HRC domain, and F1 domains I and II, (e) an HRA domain, an HRC domain, F1 domains I and II, and a heptad-repeat B domain (HRB), or (f) one or more homotrimers of F1 and F2.

3-7. (canceled)

8. The nanoparticle of claim 1, wherein the F protein comprises an amino acid sequence selected from the group consisting of SEQ ID NOs: 1-12.

9. The nanoparticle of claim 1, wherein the F protein is covalently attached or genetically fused to the self-assembling molecule.

10. (canceled)

11. The nanoparticle of claim 1, wherein the self-assembling molecule is selected from the group consisting of a protein, peptide, nucleic acid, a virus-like particle, a viral capsid, lipid, and carbohydrate.

12. The nanoparticle of claim 11, wherein the self-assembling molecule assembles into a shell with polyhedral symmetry.

13. The nanoparticle of claim 12, wherein the shell has an octahedral symmetry.

14. The nanoparticle of claim 13, wherein the shell comprises twenty four monomers of the self-assembling molecule.

15. The nanoparticle of claim 11, wherein the self-assembling molecule is a protein selected from the group consisting of ferritin, heat shock protein, and Dsp.

16. (canceled)

17. The nanoparticle of claim 15, wherein the self-assembling molecule is ferritin, wherein the ferritin comprises an amino acid sequence selected from the group consisting of SEQ ID NOs: 13-17.

18. The nanoparticle of claim 15, wherein the self-assembling molecule is a heat shock protein, selected from the group consisting of, sHSP (small heat shock protein), HSP100, HSP90, HSP70, and HSP60.

19. The nanoparticle of claim 15, wherein the self-assembling molecule is a heat shock protein, wherein the heat shock protein comprises an amino acid sequence selected from the group consisting of SEQ ID NOs: 36-42.

20. The nanoparticle of claim 1, wherein the F protein and self-assembling molecule are attached by means of a linker.

21. The nanoparticle of claim 20, wherein the linker is of sufficient length to prevent steric hindrance between the self-assembling molecule and F protein.

22. The nanoparticle of claim 20, wherein the linker is a gly-ser linker.

23. (canceled)

24. The nanoparticle of claim 1, wherein the amino acid sequence of the F protein further comprises an N-terminal leader that facilitates secretion from cells.

25. The nanoparticle of claim 24, wherein the N-terminal leader comprises an amino acid sequence selected from the group consisting of SEQ ID NOs: 51-53.

26. An immunogenic composition comprising the nanoparticle of claim 1 and a pharmaceutically acceptable carrier, optionally further comprising an adjuvant.

27. (canceled)

28. A vaccine composition comprising a nanoparticle according to claim 1, wherein F protein homotrimers in a pre-fusion conformation are displayed on the surface of a shell formed by polymeric assembly of the self-assembly molecule, optionally further comprising an adjuvant.

29. (canceled)

30. An RSV F protein-ferritin fusion protein comprising the amino acid sequence selected from the group consisting of SEQ ID NOs: 18-21, 24-27, and 30-33.

31. An RSV F protein-heat shock protein fusion protein comprising the amino acid sequence selected from the group consisting of SEQ ID NO: 22, 23, 28, 29, 34, and 35.

32. A kit comprising the nanoparticle or fusion protein of claim 1, and instructions for use.

33. A method of producing an antibody which inhibits and/or prevents RSV infection comprising administering to a subject the nanoparticle of claim 1, optionally comprising isolating the antibody from the subject.

34. (canceled)

35. A method of producing a vaccine against RSV, the method comprising a) expressing a complex comprising a monomeric self-assembly molecule and an RSV F protein under conditions such that F protein trimers in a pre-fusion conformation are displayed on the surface of a shell formed by polymerization of the self-assembly molecule, and b) recovering the shell displaying the F protein.

36. A method of vaccinating a subject against RSV comprising administering to the subject a vaccine produced according to the method of claim 35.

37. The method of claim 36, wherein the linker attachment point on the F protein is leucine at position 513 of SEQ ID NO: 1, and the linker attachment point on ferritin is aspartic acid at position 5 of SEQ ID NO: 13.

38. An isolated nucleic acid encoding the nanoparticle or fusion protein of claim 1.

39. A vector comprising the nucleic acid of claim 38.

40. An isolated cell comprising the nucleic acid of claim 39.
